(19) United States
(12) Patent Application Publication
(10) Pub. No.: US 2015/0329582 A1
(43) Pub. Date: Nov. 19, 2015

(54) SMALL MOLECULE COMPOUNDS SELECTIVE AGAINST GRAM-NEGATIVE BACTERIAL INFECTIONS

(71) Applicant: Jef DeBrabander, Flower Mound, TX (US)

(72) Inventor: Jef DeBrabander, Flower Mound, TX (US)

(21) Appl. No.: 14/711,545

(22) Filed: May 13, 2015

Related U.S. Application Data
(60) Provisional application No. 61/992,642, filed on May 13, 2014.

(51) Int. Cl. C07H 17/08; C07F 7/18; C07H 15/04; C07H 9/04; C07D 301/00; C07H 3/02; C07D 309/12; C07C 29/132; C07C 41/18; C07C 41/22; C07H 3/08; C07F 7/22; C07C 29/44

(52) U.S. Cl. CPC C07H 17/08; C07F 7/2212; C07F 7/188; C07H 15/04; C07H 9/04; C07D 301/00; C07C 29/44; C07D 309/12; C07C 29/132; C07C 41/18; C07C 41/22; C07H 3/08; C07H 3/02

(57) ABSTRACT

The described invention provides fully synthetic, biologically active mangrolide A. It describes schemes to chemically synthesize mangrolide A, intermediates and analogs of mangrolide A, and their antibacterial activity.

### CROSS-REFERENCE TO RELATED APPLICATION

The present application claims the benefit of priority to U.S. Provisional Patent Application Ser. No. 61/992,642, filed May 13, 2014, the content of which is incorporate herein by reference in its entirety.

### FIELD OF INVENTION

The described invention generally relates to small molecule compounds selective against gram-negative bacterial infections resistant to currently available antibiotics.

### BACKGROUND OF THE INVENTION

Bacterial resistance to antibiotics is increasing in both community and hospital settings, leading to higher mortality and morbidity rates. The continuous development and the spread of bacterial resistances pose questions about the future of current antibiotics and represent a serious threat for their clinical utility, leading to an urgent requirement for new compounds. (Bassetti et al., Ann Clin Microbiol Antimicrob. 2013; 12:22)

Multidrug resistant (MDR) bacterial strains are those nonsusceptible to one or more antimicrobial agents of three or more antimicrobial classes, while bacterial strains that are non-susceptible to all antimicrobials are classified as extreme drug-resistant strains. MDR bacteria have a significant impact on mortality, hospital stay, and associated costs. (Id.) However, since 2000, only three new classes of antibiotics have been introduced to the market for human use. ‘Innovation gap’ is the expression that has been used to describe the lack of novel structural classes introduced to the antibacterial armamentarium since 1962. (Id.)

The microorganisms that are principally involved in the resistance process are the ESKAPE pathogens (Enterococcus faecium, Staphyloccus aureus, Klebsiella pneumoniae, Acinetobacter baumanii, Pseudomonas aeruginosa, and Enterobacteriaceae), so named to emphasize their capacity to “escape” from common antibacterial treatments. (Id.)

### Healthcare Associated Infections

Antimicrobial-resistant pathogens that cause healthcare-associated infections (HAIs) pose an ongoing and increasing challenge to hospitals, both in the clinical treatment of patients and in the prevention of the cross-transmission of these problematic pathogens, include Gram-positive methicillin-resistant Staphylococcus aureus (MRSA), vancomycin-resistant Gram-positive Enterococcus species, extended-spectrum β-lactamase-producing gram negative Escherichia coli and Klebsiella species, and fluoroquinolone- or carbapenem-resistant Gram-negative Enterobacteriaceae or Pseudomonas aeruginosa. (Hidron et al., Infect Control Hosp Epidemiol 2008; 29:996-1011) Of the 2.0 million annual hospital-acquired bacterial infections, more than 30% are caused by Gram-negative bacteria, which account for 70% of Intensive Care Unit (ICU) infections.

### Current Therapeutic Strategies

Although many different antibiotics have been discovered or generated over the years, the current arsenal of antibiotics useful to combat Gram-negative and Gram-positive bacterial infections is limited by accumulating resistance and drug toxicity.

### 1. Inhibitors of Cell Wall Synthesis

### Penicillins

Penicillins are produced by members of the fungal genus Penicillium The basic chemical structure consists of a β-lactam ring, a thiazolidine ring, and a side chain (6-aminopenicillanic acid). (Victor L. Yu, et al., Antimicrobial Therapy and Vaccines, (1999) 1^{st} Ed., Williams & Wilkins, at 850-874):

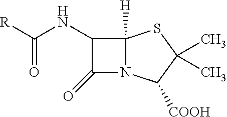

Penicillins exert their bactericidal activity primarily by inhibiting bacterial cell wall synthesis by binding to penicillin-binding proteins (PBPs), which are enzymes (transpeptidases, carboxypeptidases, and endopeptidases) that play an important role in formation and maintenance of the cell wall structure. The bacterial cell wall is made up of peptidoglycan, or murein sacculus, a polymeric component consisting of long polysaccharide chains of N-acetylglycosamine and N-acetylmuramic acid cross-linked by shorter peptide chains. Peptidoglycan is formed in three stages: precursor formation in the cytoplasm, linkage of precursor products into a long polymer, and finally cross-linking by transpeptidation. It is the final transpeptidation process that is inhibited by penicillins acting as structural analogues of acyl-D-alanyl-D-alanine (the substrate of the enzyme) and acylating the transpeptidase enzyme. The peptidoglycan structure, and therefore the cell wall structure, is weakened, leading to bacterial cell death.

Penicillin compounds can be divided into categories based upon their spectrum of activity: natural penicillins, penicillinase-resistant penicillins, aminopenicillins, carboxypenicillins, and ureidopenicillins and piperazine penicillin.

The natural penicillins (e.g., Penicillin G, Peniccilin V) have activity against non-β-lactamase-producing Gram-positive cocci, including Viridans streptococci, group A streptococci, Streptococcus pneumonia, and anaerobic streptococcus.

Penicillinase-resistant penicillins (e.g., nafcillin, oxacillin, diclosacillin), also known as the antistaphylococcal penicillins, are achieved by adding an isoxazolyl side chain to the penicillin compound, protecting the β-lactam ring from acid hydrolysis by penicillinases produced by Staphylococcus. Strains of methicillin-resistant Staphylococcus aureus (MRSA), referred to as S. aureus (MRSA), and methicillin-resistant Staphyloccus epidermidis (MRSE) prevalent in certain hospitals or wards are not sensitive to the penicillinase-resistant penicillins.

Aminopenicillins are penicillins that have an amino group added to the basic penicillin compound; their spectrum of activity against Gram-positive organisms is similar to that of the natural penicillins. These agents regain activity against streptococci and have slightly greater activity against enterocossus and Listeria monocytogenes than the natural penicillins. The enhanced spectrum of these drugs includes activity against Gram-negative bacilli, including Haemophilus influenza, Escherichia coli, Proteus mirabilis, Salmonella, and Shigella. Many strains of these Gram-negative organisms are resistant to, e.g., ampicillin.

Clinical use of penicillins is limited by the emergence of resistant organisms. Of particular concern is the emergence of penicillin-resistant (and multidrug resistant) pneumococci and methicillin-resistant staphylococci, as treatment options in these scenarios are limited. There are three main mechanisms of resistance to penicillins: (a) enzymatic degradation of the penicillin by hydrolysis of the β-lactam bond, (b) inability of the penicillin to penetrate the cell membrane to reach its target site, and (c) alteration of the penicillin-binding protein (PBP) target site by mutation.

### Cephalosporins

Cephalosporins, are semi-synthetic beta-lactam antibiotics which are derived from the fungal genus Cephalosporium, generally have a broad spectrum of action and are useful in treating various infections caused by Gram-positive and Gram-negative bacteria. They inhibit the formation of peptide cross-linkages within the peptide glycan backbone of bacterial cell walls. Without functional cell wall structures, growing bacterial cells are not protected against osmotic shock and are subject to lysis. Cephalosporins are not, however, effective against resting or dormant bacteria. (Victor L. Yu, et al., Antimicrobial Therapy and Vaccines, (1999) 1^{st} Ed., Williams & Wilkins, at 703-764)

The basic structure of a cephalosporin is:

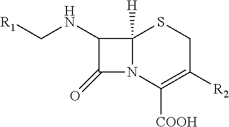

Cephalosporins are divided into three groups: cephalosporin N and C are chemically related to penicillins and cephalosporin P is a steroid antibiotic resembling fusidic acid. The mechanism of action (MOA) of cephalosporins is interference with bacterial cell wall synthesis. First generation cephalosporins include: Duricef®/cefadroxil, Velosef®/cephradine, Kefzol®/cefaxolin, Keflex®/cephalexin, Ancef®/cefazolin, Biocef®/cephalexin, and Panixine®/cephalexin. Second generation cephalosporins include: Lorabid®/loracarbef, Cefotan®/cefotetan, Ceftin®/Zinacef®/cefuroxime, Cefzil®/cefprozil, borabid Pulvules®/loracarbef, Mefoxin®/cefoxitin, and Raniclor®/cefaclor. Third generation cephalosporins include: Cedax®/ceftibuten, Rocephin®/ceftriaxone, Claforan®/cefotaxime, Vantin®/cefpodoxime, Omnicef®/cefdinir, Suprax®/cefixime, Spectracef®/cefditoren, Cefizox®/ceftizoxime, Cefobid®/cefoperazone, Ceptaz®/ceftazidime, and Fortaz®/Tazicef®/ceftazidime. Fourth generation cephalosprins include: Maxipime®/cefepime. New generation cephalosporins include: Teflaro®/ceftaroline.

### Cephalosporins: Generations 1-4 and Spectrum of Activity

Parenteral and oral cephalosporins have been grouped into generations on the basis of their antibacterial activity and spectrum of microbiologic activity. Cephems are traditionally divided into first-, second-, third-, and fourth-generation cephalosporins. The first generation compounds have a relatively narrow spectrum of activity focused primarily on Gram-positive cocci. Second-generation cephalosporins have variable activity against Gram-positive cocci but have increased activity against Gram-negative bacteria. In spite of relatively increased potency against Gram-negative aerobic and anaerobic bacilli, the cephamycins are included in the second generation. Those cephalosporins with very marked activity against Gram-negative bacteria are grouped in a third generation; some of these compounds have limited activity against Gram-positive cocci, particularly methicillin-susceptible S. aureus. Only ceftazidime and cefoperazone have clinically useful activity against Pseudomonas aeruginosa. The newest cephalosporins represent attempts to maintain activity against Gram-positive and Gram-negative organisms, including P. aeruginosa and many of the Enterobacteriaceae, and avoid many of the problems of resistance that have affected other antimicrobial compounds. These agents are the fourth-generation cephalosporins. (Victor L. Yu, et al., Antimicrobial Therapy and Vaccines, (1999) 1^{st} Ed., Williams & Wilkins, at 748-764)

Since Cephalosporins contain a β-lactam ring, penicillinase-producing bacterial strains can degrade the ring structure, rendering these antibiotics ineffective in treating such strains. However, many of them, such as cefoxitin and cepahalothin, are relatively resistant to β-lactamases.

### Carbapenems

Carbapenems are a group of naturally occurring antibiotics produced by the soil organism Streptomyces cattleya. They have the widest spectrum of antibacterial activity of all the beta-lactams and have activity against many Gram-negative and Gram-positive aerobic and anaerobic bacteria. Examples of carbapenems include: Doribax®/doripenem, Merrem®/meropenem, Invanz®/ertapenem, Primaxin®/cilastatin/imipenem. Carbapenems can be delivered orally to a patient. (Victor L. Yu, et al., Antimicrobial Therapy and Vaccines, (1999) 1^{st} Ed., Williams & Wilkins, at 690-702)

The general structure of a carbapenem is:

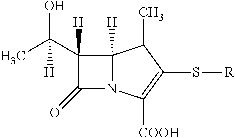

Carbapenems are bactericidal agents that bind to the penicillin-binding-proteins (PBPs) on a cell wall, inhibit the bacterial cell wall synthesis. The actions of carbapenems result in bactericidal effects through inhibition of cellular growth and division and loss of cell wall integrity, eventually causing cell lysis.

Among Gram-positive aerobes, the carbapenems are active against most strains of methicillin-susceptible Staphylococcus aureus and coagulase-negative staphylococci. Aerobic hemolytic streptococci are also highly susceptible, as is Listeria monocytogenes. Strains of Streptococcus pneumonia with intermediate- or high-level resistance to penicillin are usually susceptible to carbapenems; however, these strains are usually four-to eightfold less susceptible than fully penicillin-susceptible strains. Activity of the carbapenems against enterococci varies considerably between species. Carbapenems exhibit excellent in vitro activity against Gram-negative aerobic bacteria. Meropenem is generally slightly more active than imipenem against Gram-negative clinical isolates. The carbapenems are extremely active against both β-lactamase-positive and -negative strains of Neisseria gonorrhoeae and Haemophilus influenza, including ampicillin-resistant β-lactamase-negative Haemophilus strains. Clinical isolates of Acinetobacter are usually quite susceptible to the carbapenems.

The carbapenems are also active against most strains of Gram-positive and Gram-negative anaerobes, including Peptostreptococcus, Propionibacterium acnes, Actinomyces, and Actinobacillus. Organisms commonly considered to be minimally susceptible or resistant to the carbapenems include methicillin-resistant staphylococci, E. faecium, Stenotrophomonas (Xanthomonas) maltophilia, and Flavobacterium meningosepticum. Mycoplasma and Chlamydia are also resistant to the carbapenems.

While carbapenems are generally more resistant to β-lactamases than other (3-lactam antibiotics, because of their stability to hydrolysis by many extended-spectrum chromosomal and plasmid-mediated β-lactamases, including AmpC and extended-spectrum β-lactamases (ESBLs). (Bassetti et al., Ann Clin Microbiol Antimicrob. 2013; 12:22), they are susceptible to the New Delhi metallo-beta-lactamase (NDM-1). 26.4% of P. aeruginosa and 36.8% of A. baumanii hospital-acquired isolates have been found resistant to carbapenems. (Hidron et al., Infect. Control Hosp. Epidemiol. 2008, 29, 996.)

### Monobactams

Monobactams (monocyclic bacterially derived β-lactams) are naturally occurring antibiotic compounds produced by bacteria in soil, such as Chromobacterium, Agrobacterium, Acetobacter, Flexibacter, Gluconobacter, and even some Pseudomonas species. (Victor L. Yu, et al., Antimicrobial Therapy and Vaccines, (1999) 1^{st} Ed., Williams & Wilkins, at 831-837). The the β-lactam ring stands alone and is not fused to another ring, in contrast to most other β-lactams, which have at least two rings. The general structure for monobactams is:

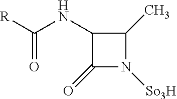

The monobactams are effective only against Gram-negative bacteria. (Bassetti et al., Ann Clin Microbiol Antimicrob. 2013; 12:22) The mechanism of action is similar to other bactericidal actions of other β-lactams, i.e., through inhibition of Gram-negative penicillin binding proteins. Thus, monobactams interfere with cell wall synthesis by binding to and inactivating penicillin-binding proteins, producing bacterial filamentation, cell lysis, and death.

Aztreonam, an exemplary monobactam, has potent activity against most aerobic Gram-negative bacteria, including Pseudomonas aeruginosa. Aztreonam is active against more than 90% of Enterobacteriaceae, including some strains that are resistant to other β-lactams and aminoglycosides. Susceptibility testing has shown that the in vitro activity of aztreonam against Escherichia coli, Proteus mirabilis, P. vulgaris, Morganella morganii, Providencia rettgeri, and Providencia stuartii, Citrobacter freundii, and Klebsiella, including K. pneumonia, consistently shows susceptibility.

Other monobactams include pirazmonam, tigemonam, and carumonam. Pirazmonam shows activity similar to aaztreonam with regard to its activity against members of the Enterobacteriaceae family but is also highly active against P. aeruginosa. Pirazmonam also shows activity against Pseudomonas and Acinetobacter.

Tigemonam is a monobactam that demonstrated bactericidal activity in vitro against members of Enterobacteriaceae family, H. influenza, Moraxella catarrhalis, and N. gonorrhoeae, including those producing β-lactamase. However, tigemonam did not inhibit Pseudomonas or Acinetobacter, and Gram-positive organisms demonstrated in vitro resistance to tigemonam.

Carumonam is highly active in vitro against Gram-negative Enterobacteriaceae, P. aeruginosa, and H. influenza, weakly active against Streptococcus pneumonia, and inactive against Staphylococcus aureus. Carumonam exhibits resistance to hydrolysis by plasmid-mediated and chromosomal β-lactamases, which shows more stability than aztreonam to the extended-spectrum β-lactamases expressed by K. pneumonia and E. cloacae; however, its activity against P. aeruginosa was intermediate between aztreonam and ceftazidime.

Resistance to monobactams can occur from intrinsic difficulty passing through the Gram-negative outer membrane, particularly with P. aeruginosa.

### β-Lactam Agent/β-Lactamase Inhibitors

In β-lactam agent/β-lactamase inhibitor combinations, the β-lactamase inhibitor potentiates the action of the β-lactam agent by protecting it from enzymatic hydrolysis. Examples include augmentin XR®/amoxicillin/clavulanate, Unasyn®/ampicillin/sulbactam, Zosyn®/piperacillin/tazobactam, Augmentin®/amoxicillin/clavulanate, augmentin ES-600, Amoclan®/amoxicillin/clavulanate, and Timentin®/clavulanate/ticarcillin. (Victor L. Yu, et al., Antimicrobial Therapy and Vaccines, (1999) 1^{st} Ed., Williams & Wilkins, at 676-689),

Exemplary lactamase inhibitors are clavulanic acid, the penam sulfones sulbactam and tazobactam, and new compounds such as BRL 42715. The structures of these four representative β-lactamase inhibitors are:

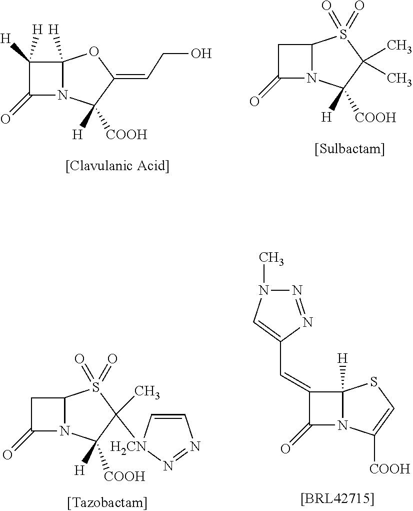

These compounds, initially recognized as normal substrates by the β-lactamase, eventually form covalent bonds with various amino acid residues within the active site, thus leading to irreversible inactivation of the enzymatic activity.

Currently used β-lactam/β-lactamase inhibitor compounds are highly active against various extended spectrum beta-lactamases. β-lactamase inhibitors generally have very little antimicrobial activity themselves, but rather, typically restore antimicrobial activity to other β-lactams.

In the last decade, the clinical usefulness of β-lactamase inhibitors has been compromised by emergence of isolates resistant to clavulanic acid. Clavulanate resistance in E. coli is increasingly associated with overproduction of the wild-type penicillinases TEM-1 and TEM-2 or with production of β-lactamases showing low sensitivity to β-lactamase inhibitors, e.g., the IRT and the OXA enzymes. Resistance to amoxicillin-clavulanate in clinical isolates producing inhibitor-resistant TEM enzymes results from point mutations that alter the region of the genes encoding TEM-1 and TEM-2. Such substitutions, initially described in E. coli, have been reported in Klebsiella pneumonia and Proteus mirabilis. The mutations are located in or near the active site of the enzyme.

### 2. Inhibitors of Protein Synthesis

Inhibitors of protein synthesis are inhibitors that specifically target bacterial 70S ribosomes, thereby selectively blocking bacterial protein synthesis.

### Aminoglycosides

Aminoglycosides are produced by actinomycetes. For example, streptomycin is produced by Streptomyces griseus, neomycin by Streptomyces fradiae, kanamycin by Streptomyces kanamyceticus, and gentamycin by Micromonospora purpurea. Amikacin is a semisynthetic derivative of kanamycin. The aminoglycoside antibiotics are composed of modified amino-sugar residues. Exemplary structures of amikacin, gentamicin Cla, and kanamycin B are:

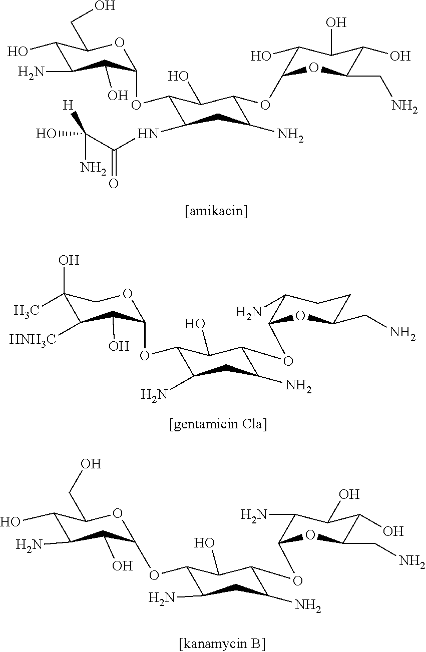

Aminoglycosides are used almost exclusively in the treatment of infections caused by Gram-negative bacteria, and are relatively ineffective against anaerobic bacteria, facultative anaerobes growing under anaerobic conditions, and Gram-positive bacteria. Nine (gentamicin, tobramycin, amikacin, streptomycin, neomycin, kanamycin, paromomycin, netilmicin, and spectinomycin) are approved for clinical use in the United States and Europe. (Victor L. Yu, et al., Antimicrobial Therapy and Vaccines, (1999) 1^{st} Ed., Williams & Wilkins, at 621-637)

Aminoglycosides bind to the 30S subunit of bacterial ribosomes, blocking protein synthesis and decreasing the fidelity of translation of the genetic code. They disrupt the normal functioning of the ribosomes by interfering with the formation of initiation complexes, the first step of protein synthesis that occurs during translation, and induce misreading of mRNA, leading to formation of nonfunctional enzymes. The interference with protein synthesis results in the death of the bacterium. Various mutations can occur, however, that reduce the effect of misreading some mRNA molecules.

To be effective, aminoglycoside antibiotics must be transported across the cytoplasmic membrane. Although sensitive bacteria transport the aminoglycosides across the cytoplasmic membrane, accumulating these antibiotics intracellularly, resistant strains may lack a mechanism for aminoglycoside transport into the cell. Resistant strains also may produce enzymes that degrade or transform the aminoglycoside molecules, for example, various enzymes associated with the plasma membranes of some bacterial strains can adenylate, acetylate, or phosphorylate aminoglycosides. Also mutations can occur that alter the site at which the aminoglycosides normally bind to the bacterial ribosomes. Some Pseudomonas aeruginosa strains, for example, possess ribosomes to which streptomycin is unable to bind. Relative to other classes of antibiotics, the aminoglycosides have demonstrated relative stability against the development of resistance. Treatment-emergent resistance (especially when used in combination with other agents) is rare. (Bassetti et al., Ann Clin Microbiol Antimicrob. 2013; 12:22.)

Since the introduction of aminoglycoside antibiotics into clinical practice, the major obstacle limiting their use is the potential for drug-related ototoxicity and nephrotoxicity.

Streptomycin is used in the treatment of only a limited number of bacterial infections because of its serious side effect on the 8^{th} cranial nerve, resulting in deafness with prolonged usage. Gentamycin, while effective, is extremely toxic and is thus used only in severe infections that may prove lethal if unchecked, particularly when the infecting bacteria are not sufficiently sensitive to other, less toxic antibiotics. Tobramycin has properties similar to those of gentamicin, but Ps. aeruginosa is particularly sensitive to tobramycin. Tobramycin is administered by intravenous or intramuscular delivery. Tobramycin is susceptible to inactivating modifications, such as phosphorylation, leading to bacterial resistance. Neomycin is primarily used in topical application. Kanamycin, a narrow spectrum antibiotic, is frequently used by pediatricians for infections due to Klebsiella, Enterobacter, Proteus and E. coli. Amikacin, which has the broadest spectrum of activity of the aminoglycosides, is the antibiotic of choice for treating serious nosocomial infections caused by Gram-negative bacteria, because such infections often are due to bacterial strains that are resistant to multiple antibiotics, including other aminoglycosides.

### Tetracyclines

The tetracyclines are a class of broad-spectrium bacteriostatic antibiotics derived from cultures of Streptomyces bacteria, and are effective as antibacterial agents by inhibiting protein synthesis after uptake into susceptible organisms. Examples of tetracyclines include: Monodox®/Oraxyl®/Doryx®/doxy100®/doxy200®/Adoxa®/Oracea®/Monodox®/Vibramycin®/Alodox®/Avidoxy®/Morgidox®/Ocudox®/Periostat®/Uracil®/vibra-Tabs®/doxycycline, Declomycin®/demeclocycline, Dynacin®/Solodyn®/Minocin®/Myrac®/Ximino®/minocycline, Terramycin®/oxytetracycline, and Actisite®/ala-Tet®/Sumycin®/tetracycline. (Victor L. Yu, et al., Antimicrobial Therapy and Vaccines, (1999) 1^{st} Ed., Williams & Wilkins, at 981-994)

A tetracycline is generally composed of four fused cyclic rings:

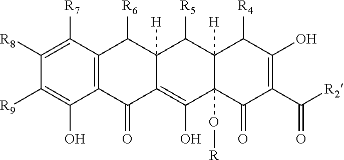

Exemplary tetracyclin compounds, such as aureomycin (produced by Streptomyces aureofaciens), demyeclocycline (also produced by Streptomyces aureofaciens), and oxytetracycline (produced by Streptomyces rimosus), are naturally occurring. Methacycline, doxycycline, minocycline, and tetracycline are all semisynthetic derivatives.

Tetracyclines bind specifically to the 30S ribosomal subunit, and block the receptor site for the attachment of aminoacyl tRNA to the mRNA ribosome complex, thus preventing addition of amino acids to a growing peptide chain.

Tetracyclines exhibit a broad spectrum of activity, which includes many aerobic and anaerobic Gram-positive and Gram-negative bacteria. They also possess activity against other microbes such as Rickettsiae, Coxiella burnetii, Borrelia recurrentis, Borrelia burgdorferi, Treponema pallidum, Treponema pertenue, Chlamydia, Mycoplasma pneumoniae, Plasmodium, Entamoeba histolytica, and Mycobacterium marinum. Tetracyclines have negligible activity against fungi and viruses. Doxycycline and minocycline are considered the most active of the tetracyclines, followed by tetracycline.

Sensitivity to tetracyclines depends on the transport of the tetracycline molecules across the cytoplasmic membrane. Some tetracyclines, such as doxycycline, appear to pass directly across the membrane, while others enter the bacterial cell only by active transport. Resistance to tetracyclines develops because of the movement of a transposon between a plasmid and the bacterial chromosome, and involves an alteration of the mechanisms of membrane transport of the tetracycline molecules. Notably, once microorganisms develop resistance to one tetracycline, this typically confers resistance to the class of drugs. Most commonly, resistance occurs when less tetracycline accumulates within the cell. This may occur in two ways. Influx of tetracycline into the cell may decrease or, more likely, efflux of tetracycline from the cell may increase. Tetracycline influx is altered by chromosomal mutations that change the outer membrane pore through which tetracycline diffuses. This alteration also confers low-level resistance to β-lactams, chloramphenicol, and quinolones. Then, when the organism is exposed to tetracycline or chloramphenicol, there is selection for mutations that confer high-level resistance to tetracycline or chloramphenicol, as well as structurally unrelated drugs such as the penicillins, cephalosporins, and quinolones. For tetracycline, high-level resistance is accomplished by antibiotic efflux, mediated by Tet membrane proteins, resulting in energy-dependent pumping of the drug from the cell. Thus, tetracycline is prevented from accumulating inside the cell, which ultimately protects the ribosome from inhibition.

### Macrolides

Macrolides are antibiotic compounds produced by streptomycetes. They are natural lactones with a large ring, consisting of 14 to 20 atoms. Macrolides commercially available or in clinical development can be divided into 14-, 15-, and 16-membered lactone ring macrolides. (Leclercq and Courvalin, Antimicrobial Agents and Chemotherapy, 1991, 35(7): 1267-1272) The only compound with a 15-membered ring, azithromycin, contains a tertiary amino group. Macrolides bind to the 50S subunit of the bacterial ribosome and inhibit ribosomal translocation, leading to inhibition of bacterial protein synthesis. Their action is primarily bacteriostatic but can be bactericidal at high concentrations, or depending on the type of microorganism. Macrolides mainly affect Gram-positive cocci and and intracellular pathogens such as mycoplasma, chlamydia, and legionella. Examples of macrolides are: Dificid®/fidaxomicin, erythrocin stearate FilmTab®/ery-Tab®/Eryc®/Eryped®/Erythrocin®/erythrocin Lactobionate®/Ilosone®/PCE disperTab®/erythromycin, Zithromax®/Zmax®/azithromycin, biaxin XL®/clarithromycin, and Tao®/troleandomycin. (Victor L. Yu, et al., Antimicrobial Therapy and Vaccines, (1999) 1^{st} Ed., Williams & Wilkins, at 795-819)

Erythromycin, which is produced by Streptomyces erythreus, contains a multimembered lactone ring attached to deoxy sugar moieties:

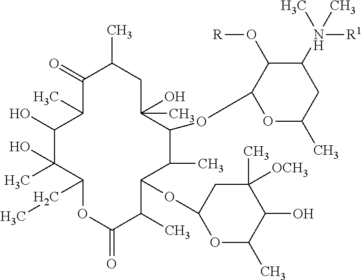

In general, the macrolides show fairly uniform activity against streptococci and methicillin-susceptible staphylococci. Clarithromycin is the most active against Bacteroides species but is least active against Haemophilus influenzae; when clarithromycin is combined with its metabolite, it has additive activity against H. influenzae. Azithromycin is most active against H. influenzae, Moraxella catarrhalis, Neisseria gonorrhoeae, and Fusobacterium species. Al macrolides have excellent activity against Bordetella pertussis. Clarithromycin has excellent activity against Mycobacterium avium complex. Azithromycin has excellent activity against Chlamydia species, Legionella species, and Mycoplasma pneumoniae. There is also direct evidence of antipseudomonal activity of the macrolides. Erythromycin is most effective against Gram-positive cocci, such as Streptococcus pyogenes. It is not active against most aerobic gram-negative rods, but does show antibacterial activity against some gram negative organisms such as Pasteurella multocida, Bordetella pertussis, and Legionella pneumophilia.

Erythromycin and other macrolides bind to the 50S ribosomal subunits of prokaryotic ribosomes with a specific target in the 23S ribosomal RNA molecule and in various ribosomal proteins, thereby inhibiting t protein synthesis. Bacteria predominantly become resistant to macrolides through mutations in 23 rRNA, the same as nonmethylated rRNA. (Giedraitiene et al., Medicina (Kaunas) 2011; 47(3): 137-46.) A susceptibility study conducted in 2000-2001 showed that 30% of all S. pneumoniae isolates were resistant to macrolides. (Stratton, Emerging Infectious Diseases, 2003 9(1): 10-16)

Macrolides are considered among the safest antimicrobial agents, and serious adverse effects are rare. Gastrointestinal adverse effects are the most common.

### Lincomycin

Linomycin is produced by Streptomyces lincolnensis. Clindamycin is a semisynthetic derivative of lincomycin.

The structure of lincomycin is:

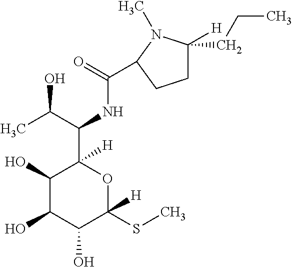 [SMILES: CCC[C@@H]1CCC(C(=O)N[C@@H]([C@H]2O[C@H](SC)[C@H](O)[C@@H](O)[C@H]2O)[C@@H](C)O)N1C]

The structure of clindamycin is:

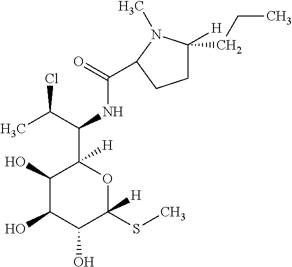 [SMILES: CCC[C@@H]1CCC(C(=O)N[C@@H]([C@H]2O[C@H](SC)[C@H](O)[C@@H](O)[C@H]2O)[C@@H](C)Cl)N1C]

Clindamycin and lincomycin inhibit bacterial protein synthesis by binding to the 50S ribosomal bacterial subunit, and block protein synthesis by inhibiting initiation of peptide chain synthesis.

Clindamycin and lincomycin are particularly effective against Gram-positive bacteria, including anaerobes, and in the treatment of infections due to Bacteroides and Fusobacterium species. Although clindamycin is regarded primarily as a bacteriostatic agent, the drug has a concentration-dependent bactericidal activity against a variety of organisms, including Staphylococci, Streptocci, anaerobes, and H. pylori. (Victor L. Yu, et al., Antimicrobial Therapy and Vaccines, (1999) 1^{st} Ed., Williams & Wilkins, at 774-788)

Resistance to lincomycin is common, and leads to co-resistance with macrolide, lincosamide and streptogramin B antibiotics. It is usually the result of modification of the target receptor with the result that the antibiotic is unable to bind. Specifically, the 50S ribosomal subunit RNA is methylated and the 23S subunit is also altered. The gene causing this change is plasmid mediated and encoded on transposons. In addition, enzymatic inactivation of lincomycin by conversion into its 3-(5′-adenylate) form by clinical isolates of strains of Staphylococcus aureus and S. haemolyticus with retention of sensitivity to macrolides and streptogramins has been observed.

### Bacterial and Mammalian Inhibitors of Protein Synthesis

Other antibacterial agents that inhibit protein synthesis are not useful in treating bacterial infections because they inhibit protein synthesis in mammalian cells to the same extent as in bacterial cells. Examples of such agents are puromycin, dactinomycin (actinomycin D), and rifampin, a semi-synthetic derivative of rifamycin B.

### 3. Inhibitors of Membrane Function

### Polymyxin

Polymyxins are produced by non-ribosomal peptide synthetase systems in Gram-positive bacteria such as Paenibacillus polymyxa. Their general structure consists of a cyclic peptide with a long hydrophobic tail:

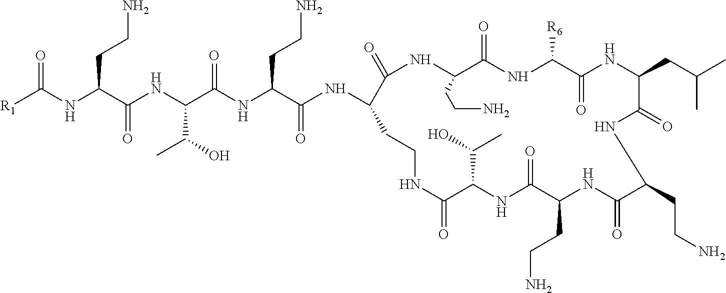

Polymyxin B: where

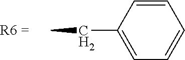

Colistin/Polymyxin E, where

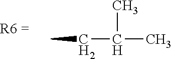

The polymyxins are selectively toxic for Gram-negative bacteria due to their specificity for the lipopolysaccharide molecule that exists within many Gram-negative outer membranes. They target the structure of the bacterial cell membrane by interacting with its phospholipids, and disrupt this structure by causing changes in the bacterial cytoplasmic membrane and leakage of cell contents. (J. Med. Chem. (2010) 11; 53(5): 1898-1916.)

Sensitive bacteria take up more polymyxin B than resistant strains. Currently, polymyxins are being used as last-line antibiotics for otherwise untreatable serious infections, such as P. aeruginosa, A. baumannii, and K. pneumoniae. However, resistance to polymyxins in hospitalized patients has been increasingly reported.

Gram-negative bacteria become resistant to polymyxins by modifying the lipid head groups in the lipopolysaccharide molecule of their cell membranes, reducing the initial electrostatic interaction between the outer membrane and polymyxins.

### 4. DNA Inhibitors

### Quinolones

The quinolone class of synthetic, bactericidal antibacterial agents has broad-spectrium activity. Examples of quinolones are: nalidixic acid, ciprofloxacin, ofloxacin, norfloxacin, lomefloxacin, enoxacin, levofloxacin, sparfloxacin, trovafloxacin, and grepafloxacin. (Victor L. Yu, et al., Antimicrobial Therapy and Vaccines, (1999) 1^{st} Ed., Williams & Wilkins, at 875-900)

The basic structure of the quinolone molecule is:

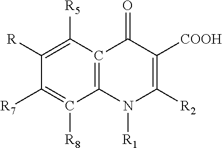

The quinolone class of antimicrobial agents targets Topoisomerase II and IV. Topoisomerase II is a DNA gyrase that is necessary for the replication of the microorganism's DNA. It nicks double-stranded chromosomal DNA, introduces negative supercoils on DNA, permitting transcription or replication, and then seals the nicked DNA. By inhibiting this enzyme, DNA replication and transcription is blocked. Topoisomerase IV is involved in decatenation of the linked DNA molecules in the bacterial cell.

The antimicrobial activity of the early, first-generation quinolones (i.e., nalidixic acid, oxolinic acid, cinoxacin, piromidic acid, pipemidic acid, and flumequine) was excellent against aerobic, gram-negative bacteria. However, first-generation quinolones were not very active against aerobic, gram-positive bacteria or anaerobic bacteria. The second-generation quinolones were introduced when norfloxacin was synthesized by adding a fluorine at C-6 and a cyclic diamine piperazine at C-7. These changes added antimicrobial activity against aerobic gram-positive bacteria and improved activity against gram-negative bacteria, compared with the first-generation compounds, but the second-generation quinolones still lacked activity against anaerobic bacteria. Norfloxacin was the first of the “fluoroquinolones,” a name resulting from the addition of a fluorine at the C-6 position. Other second-generation quinolones include ciprofloxacin, ofloxacin, levofloxacin, enoxacin, fleroxacin, lomefloxadin, pefloxacin, and rufloxacin. Newer fluoroquinolones (i.e., third-generation fluoroquinolones, including grepafloxacin, gatifloxacin, sparfloxacin, temafloxacin, tosufloxacin, and pazufloxacin) were subsequently developed and had greater potency against gram-positive bacteria, particularly pneumococci; they also had good activity against anaerobic bacteria. The fourth-generation fluoroquinolones (e.g., trovafloxacin, clinafloxacin, sitafloxacin, moxifloxacin, and gemifloxacin) had potent activity against anaerobes and increased activity against pneumococci. (Clinical Infectious Diseases (2005); 41:S113-9)

The clinical utility of quinolones has diminished due to the widespread of quinolone resistance, especially in Gram-negative rods. Bacterial resistance to quinolones occurs either with the induction of amino acid changes in specific areas of the parC and parE genes of topoisomerase IV, particularly in pneumococci, and in the gyrA gene of topoisomerase II, in staphylococci, or with amino acid changes in both topoisomerases II and IV in many bacterial species.

Marine actinomycetes and marine myxobacteria have been cultivated from sediments collected in the Caribbean and Gulf of Mexico to generate a natural product freation library of greater than 2000 fractions for biological screening. Using this library, five compounds from two marine actinomycetes were identified with potent and selective activity against the opportunistic bacterial pathogens Pseudomonas aeruginosa and Burkholderia cepacia. Two polyketides, mangrolide A and mangrolide B were identified. Mangrolide A is a macrolide antibiotic with potent and selective antibiotic activity against Gram-negative pathogens. This invention describes schemes to chemically synthesize mangrolide A, intermediates and analogs of Mangrolide A and their antibacterial activity.

### SUMMARY OF INVENTION

According to one aspect, the described invention provides synthetic, biologically active mangrolide A compound. According to one embodiment, the compound is of structural formula 14.5:

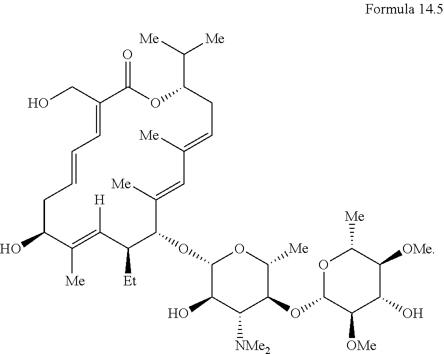 [SMILES: CC[C@H]1/C=C(\C)[C@@H](O)C/C=C/C=C(\CO)C(=O)O[C@H](C(C)C)C/C=C(C)/C=C(\C)[C@@H]1O[C@@H]1O[C@H](C)[C@@H](O[C@@H]2O[C@H](C)[C@@H](OC)[C@H](O)[C@H]2OC)[C@H](N(C)C)[C@H]1O]

According to another aspect, the described invention provides a method for treating a disease or a disorder caused by a bacterial infection in a patient, comprising administering to the patient a therapeutic amount of the synthetic compound of any one of claims 1 and 2. According to one embodiment of the method, the synthetic compound according to claim 1 exhibits antibacterial activity against a population of Gram-negative bacteria. According to another embodiment, the antibacterial activity is a bacteriocidal activity or bacteriostatic activity. According to another embodiment, the therapeutic amount of the synthetic compound according to claim 1 is effective against a multidrug resistant bacterial infection. According to another embodiment, the population of Gram-negative bacteria is a population selected from the group consisting of Enterococcus faecalis, Escherichia coli, Klebsiella pneunomiae, Burkholderia cepacia, and Pseudomonas aeruginosa. According to another embodiment, the population of Gram-negative bacteria is selected from the group consisting of Pseudomonas aeruginosa and Burkholderia cepacia.

According to another aspect, the described invention provides a pharmaceutical composition comprising a therapeutic amount of at least one synthetic compound according to any one of claims 1 and 2 and a pharmaceutically acceptable carrier.

According to another aspect, the described invention provides a method for the synthesis of a fragment of Formula 1.6:

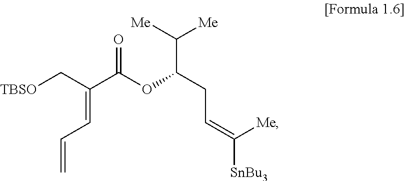

or a pharmaceutically acceptable salt thereof required for synthesis of a correct mangrolide Aglycon disilylether, comprising, in order: (a) reacting a compound of Formula 1.1:

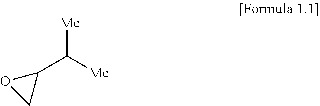

with acetic acid, toluence in the presence of a (R, R)-Salen-Co (II) catalyst at room temperature to form a compound of Formula 1.2:

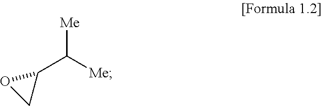

(b) reacting the compound of formula 1.2 in the presence of 1-propyne, n-butyllithium (n-BuLi) boron trifluoride diethyl etherate (BF₃OEt₂), and tetrahydrofuran (THF) at −78° C. to form a compound of Formula 1.3:

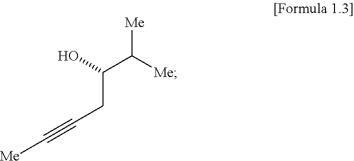

(c) reacting the compound of formula 1.3 in the presence of tributyl tin hydride (Bu₃SnH, which catalyzes the deoxygenation of alcohols), copper (I) cyanide (CuCN), n-butyllithium (n-BuLi), methanol, and tetrahydrofuran (THF), at −78° C. to −20° C., to form a compound of Formula 1.4,

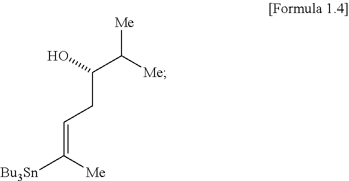

and (d) reacting the compound of formula 1.4 comprising an SnBu₃ protecting group, and a compound of Formula 1.5 comprising a tert-butyl dimethyl silyl (TBSO) protecting group,

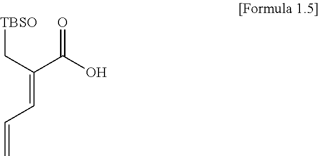 [SMILES: C=C/C=C(\CO[Si](C)(C)C(C)(C)C)C(=O)O]

in the presence of diphenyl phosphate chlorate (PhO)₂P(O)Cl), triethylamine (NEt₃), and 4-dimethyl aminopyridine (DMAP), a nucleophilic catalyst for esterification, in toluene at 45° C. to form a compound of Formula 1.6:

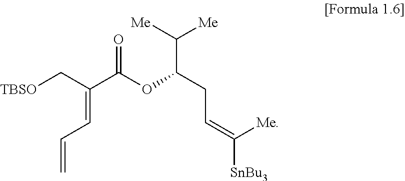

According to another aspect, the described invention provides a method for the synthesis of a compound of Formula 7.11:

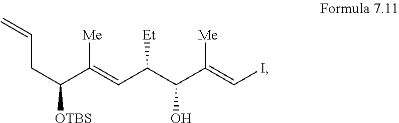 [SMILES: C=CC[C@H](O[Si](C)(C)C(C)(C)C)/C(C)=C/[C@H](CC)[C@@H](O)/C(C)=C/I]

comprising in order: (a) reacting a compound of formula 2.1 in the presence of 3,4-dihydropyran, pyridinium p-toluene sulfonate (PPTS) and dichloromethane (CH₂Cl₂) to form a compound of Formula 2.2:

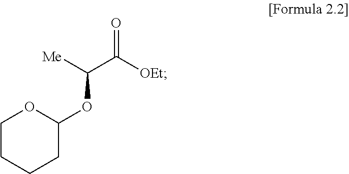 [SMILES: CCOC(=O)[C@H](C)OC1CCCCO1]

(b) reacting the compound of Formula 2.2 in the presence of diisobutylaluminum hydride (DIBAL) amd dichloroethane (CH₂Cl₂) at −78° C. to form a compound of Formula 2.3:

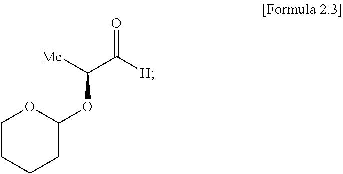

(c) reacting the compound of Formula 2.3 in the presence of n-PrPh₃PBr, n-butyllilthium (nBuLi), tetrohydrofuran (THF) at −78° C. to room temperature to form a compound of Formula 2.4:

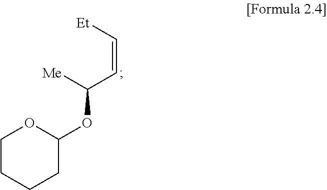 [SMILES: CC/C=C\[C@H](C)OC1CCCCO1]

(d) reacting the compound of Formula 2.4 with tosylic acid (TsOH.H₂)), ethylene glycol (HO(CH₂)2OH at 0° C. to form a compound of Formula 2.5:

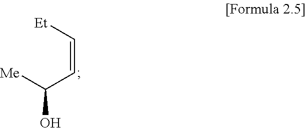

(e) reacting the compound of Formula 2.5 with a compound of formula J:

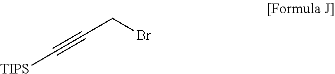

in the presence of sodium tert-butoxide (t-BuONa) and tetrahydrofuran (THF) at 0° C. to form a compound of Formula 2.6:

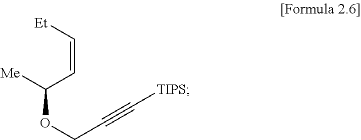

(f) reacting the compound of Formula 2.6 in the presence of n-butyllithium, tetrahydrofuran (THF) at −78° C. and then methoxy methylether chloride (MOMCl) to form a compound of Formula 7.1:

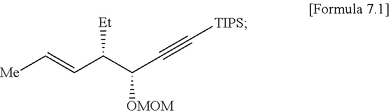

(g) reacting the compound of Formula 7.1 in the presence of ozone (03), dichloromethane (CH₂Cl₂) at −78° C.; then dimethylsulfide (Me₂S) at room temperature to form a compound of Formula 7.2:

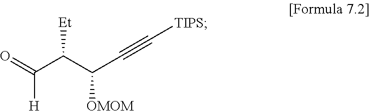

(h) reacting the compound of Formula 7.2 in the presence of Ph₃P═C(Me)CO₂Et and toluene at 90° C. to form a compound of Formula 7.3:

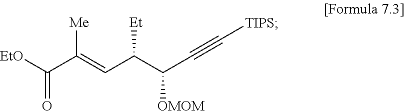

(i) reacting the compound of Formula 7.3 in the presence of diisobuutylaluminum hydride (DIBAL) and dichloromethane (CH₂Cl₂) at −78° C. to form a compound of Formula 7.4:

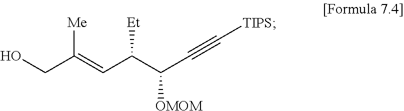

(j) reacting the compound of Formula 7.4 in the presence of tetra-n-butylammonium fluoride (TBAF) and tetrahydrofuran (THF) at 0° C. to form a compound of Formula 7.5:

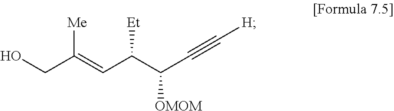

(k) reacting the compound of Formula 7.5 in the presence of triethylsilyltrifluoromethane sulfonate (TESOTf), and 2,6-lutidine at 0° C. to form a compound of Formula 7.6:

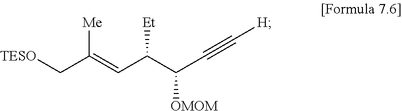

(l) reacting the compound of Formula 7.6 in the presence of tributyltin hydride (Bu₃SnSnBu₃), butyllithium (BuLi), copper (I) cyanide (CuCN), tetrahydrofuran (THF), then methyl iodide (MeI) at −78° C. to room temperature, then elemental iodine (I₂) and dichloromethane (CH₂Cl₂) at 0° C. to form a compound of Formula 7.7:

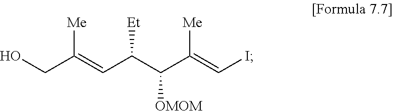

(m) reacting the compound of Formula 7.7 in the presence of Dess-Martin periodinane and dichloromethane (CH₂Cl₂) at 0° C. to form a compound of Formula 7.8:

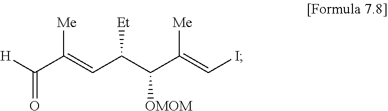

(n) reacting the compound of Formula 7.8 in the presence of (−)-lpc₂B-Allyl diethylether at −78° C. to form the compound of Formula 7.9:

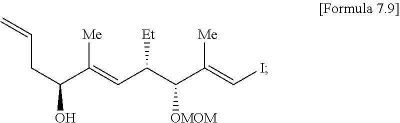

(o) reacting the compound of Formula 7.9 in the presence of triethylsilyltrifluoromethane sulfonate (TESOTf), and 2,6-lutidine at 0° C. to form a compound of Formula 7.10:

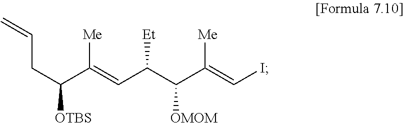

and (p) reacting the compound of Formula 7.10 in the presence of boron trifluoride etherate (BF₃.OEt₂ and dimethyl sulfide to form a compound of Formula 7.11:

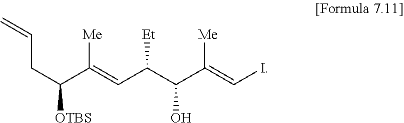 [SMILES: C=CC[C@H](O[Si](C)(C)C(C)(C)C)/C(C)=C/[C@H](CC)[C@@H](O)/C(C)=C/I]

According to another aspect, the described invention provides a method for the synthesis of a compound of Formula 8.5:

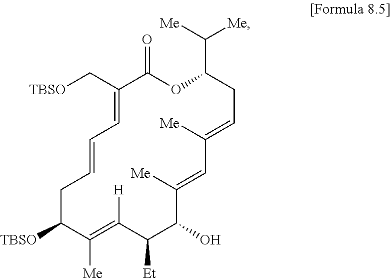 [SMILES: CC[C@H]1/C=C(\C)[C@@H](O[Si](C)(C)C(C)(C)C)C/C=C/C=C(\CO[Si](C)(C)C(C)(C)C)C(=O)O[C@H](C(C)C)C/C=C(C)/C=C(\C)[C@@H]1O]

wherein the compound of Formula 8.5 is synthetic mangrolide Aglycon disilyl ether of correct structure, comprising, in order: (a) reacting a compound of Formula 1.6:

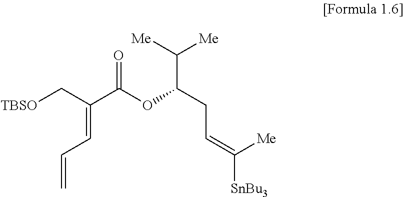

and a compound of Formula 7.11:

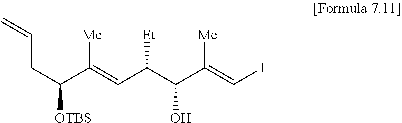 [SMILES: C=CC[C@H](O[Si](C)(C)C(C)(C)C)/C(C)=C/[C@H](CC)[C@@H](O)/C(C)=C/I]

in the presence of the catalyst tetrakis(triphenylphosphine)palladium (Pd(PPh₃)₄), CuCl, LiCl, and dimethylsulfoxide (DMSO) at 70° C. to form a compound of Formula 8.4:

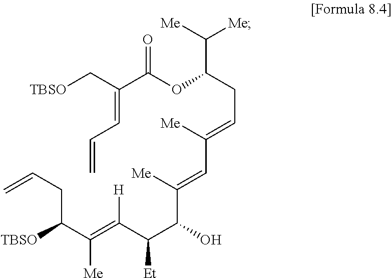 [SMILES: C=C/C=C(\CO[Si](C)(C)C(C)(C)C)C(=O)O[C@@H](C/C=C(C)/C=C(\C)[C@H](O)[C@H](/C=C(\C)[C@H](CC=C)O[Si](C)(C)C(C)(C)C)CC)C(C)C]

and (b) reacting the compound of formula 8.4 in the presence of Grubbs 2^{nd} catalyst and dichloromethane (CH₂Cl₂) under reflux conditions to form the compound of Formula 8.5:

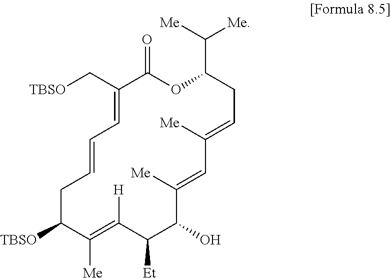 [SMILES: CC[C@H]1/C=C(\C)[C@@H](O[Si](C)(C)C(C)(C)C)C/C=C/C=C(\CO[Si](C)(C)C(C)(C)C)C(=O)O[C@H](C(C)C)C/C=C(C)/C=C(\C)[C@@H]1O]

According to another aspect, the described invention provides a method for the synthesis of a mangrolide A deoxyglucose fragment a method for the synthesis of a mangrolide A deoxyglucose fragment of Formula 10.9:

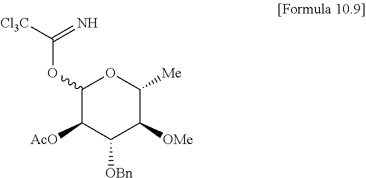

comprising, in order: (a) reacting a compound of Formula 10.1 in the presence of p-toluenesulfonyl chloride (TsCl), Pyridine (Py) and dichloromethane (CH₂Cl₂) to form a compound of formula 10.2:

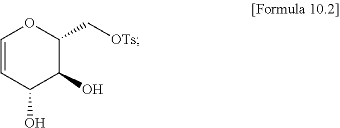 [SMILES: Cc1ccc(S(=O)(=O)OC[C@H]2OC=C[C@@H](O)[C@@H]2O)cc1]

(b) reacting the compound of Formula 10.2 in the presence of lithium aluminum hydride (LiAlH₄) and tetrahydrofuran (THF) to form a compound of Formula 10.3:

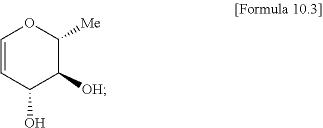

(c) reacting the compound of Formula 10.3 in the presence of dibenzyl tin (II) oxide (Bn₂SnO), and then tetrabutylammonium iodide (TBAI) and benzyl bromide (BnBr) to form a compound of Formula 10.4:

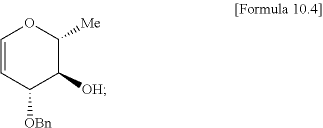 [SMILES: C[C@H]1OC=C[C@@H](OCc2ccccc2)[C@@H]1O]

(d) reacting the compound of formula 10.4 in the presence of potassium bis(trimethylsilyl)amide (KHMDS), methyl iodide (MeI) and tetrahydrofuran (THF) at −78° C. to form a compound of Formula 10.5:

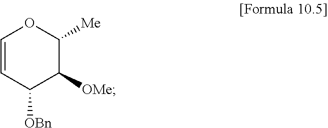 [SMILES: CO[C@@H]1[C@@H](C)OC=C[C@H]1OCc1ccccc1]

(e) reacting the compound of Formula 10.5 in the presence of osmium tetroxide (OsO₄), N-methylmorpholine-N-oxide (NMO), and tert-butanol/acetone/water (1:1:1) at room temperature to form a compound of Formula 10.6;

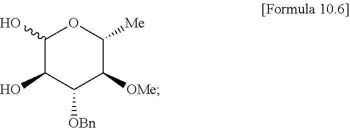 [SMILES: CO[C@H]1[C@H](OCc2ccccc2)[C@@H](O)C(O)O[C@@H]1C]

(f) reacting the compound of Formula 10.6 in the presence of acetic anhydride (Ac₂O), triethylamine (Et3N), 4-(dimethylamino)pyridine (DMAP), and dichloromethane (CH₂Cl₂) to form a compound of Formula 10.7:

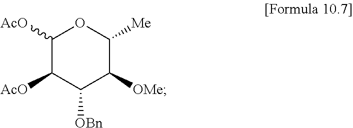 [SMILES: CO[C@H]1[C@H](OCc2ccccc2)[C@@H](OC(C)=O)C(OC(C)=O)O[C@@H]1C]

(g) reacting the compound of Formula 10.7 in the presence of hydrazine acetate and dimethylformamide (DMF) to form a compound of Formula 10.8:

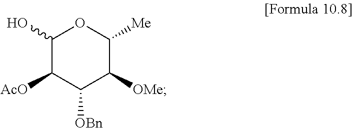 [SMILES: CO[C@H]1[C@H](OCc2ccccc2)[C@@H](OC(C)=O)C(O)O[C@@H]1C]

and (h) reacting the compound of Formula 10.8 in the presence of 1,8-diazabicycloundec-7-ene (DBU), trichloroacetonitrile (CCl₃CN) and dichloromethane (CH₂Cl₂) to form the compound of Formula 10.9:

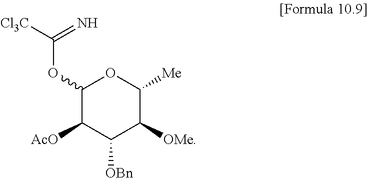

According to another aspect, the described invention provides a method for the synthesis of a mangrolide A mycaminose sugar of Formula 11.3:

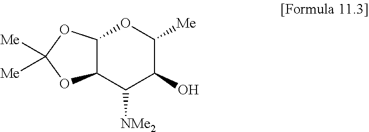

comprising, in order: (a) reacting a compound of Formula 11.1:

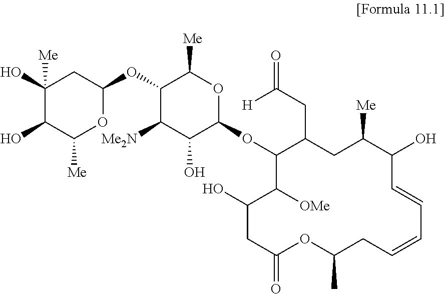 [SMILES: COC1C(O)CC(=O)O[C@H](C)C/C=C\C=C\C(O)[C@H](C)CC(CC=O)C1O[C@@H]1O[C@H](C)[C@@H](O[C@@H]2C[C@](C)(O)[C@H](O)[C@@H](C)O2)[C@H](N(C)C)[C@H]1O]

in the presence of 2N HCl under reflux conditions, to form a compound of Formula 11.2:

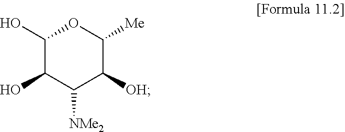

and (b) reacting the compound of formula 11.2 in the presence of

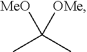

pyridinium toluene-4-sulfonate (PPTS) and dimethylformamide (DMF) to form the compound of Formula 11.3:

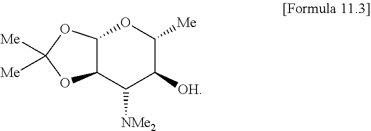

According to another aspect, the described invention provides a method for the synthesis of a disaccharide fragment of mangrolide A of Formula 12.6:

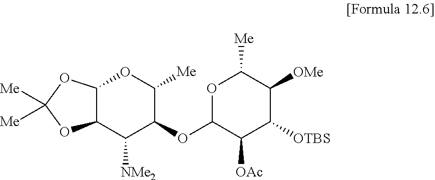 [SMILES: CO[C@H]1[C@H](O[Si](C)(C)C(C)(C)C)[C@@H](OC(C)=O)C(O[C@H]2[C@H](N(C)C)[C@H]3OC(C)(C)O[C@@H]3O[C@@H]2C)O[C@@H]1C]

comprising, in order: (a) reacting a compound of Formula 10.3

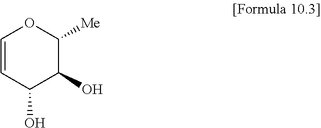

in the presence of tert-butyldimethylsilyl chloride (TBSCl), imidazone, and dichloromethane (CH₂Cl₂) to form a compound of Formula 12.1:

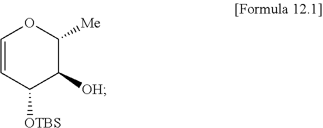 [SMILES: C[C@H]1OC=C[C@@H](O[Si](C)(C)C(C)(C)C)[C@@H]1O]

(b) reacting the compound of Formula 12.1 in the presence of potassium bis(trimethylsilyl)amide (KHMDS), methyl iodide (MeI) and tetrahydrofuran (THF) at −78° C. to form a compound of Formula 12.2:

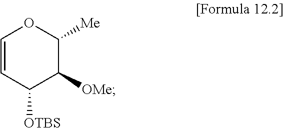 [SMILES: CO[C@@H]1[C@@H](C)OC=C[C@H]1O[Si](C)(C)C(C)(C)C]

(c) reacting the compound of Formula 12.2 in the presence of osmium tetroxide (OsO₄), N-methylmorpholine-N-oxide (NMO), and tert-butanol/acetone/water (1:1:1) at room temperature to form a compound of Formula 12.3:

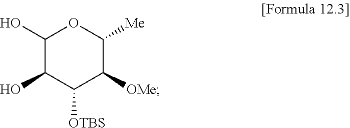 [SMILES: CO[C@H]1[C@H](O[Si](C)(C)C(C)(C)C)[C@@H](O)C(O)O[C@@H]1C]

(d) reacting the compound of Formula 12.3 in the presence of acetic anhydride (Ac2O), triethylamine (Et3N), 4-(dimethylamino)pyridine (DMAP), and dichloromethane (CH2Cl2) to form a compound of Formula 12.4:

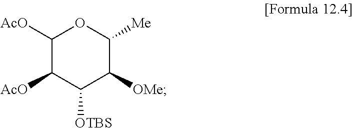 [SMILES: CO[C@H]1[C@H](O[Si](C)(C)C(C)(C)C)[C@@H](OC(C)=O)C(OC(C)=O)O[C@@H]1C]

(e) reacting the compound of Formula 12.4 with a compound of Formula 11.3:

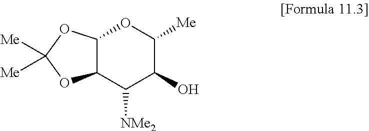

in the presence of tin (IV) chloride (SnCl4) at −15° C. to room temperature to form a compound of Formula 12.5:

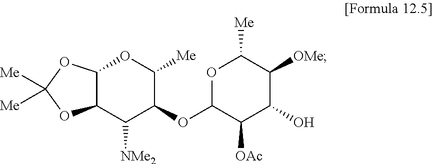

and (f) reacting the compound of Formula 12.5 in the presence of trifluoromethylsilyl trifluoromethane sulfonate (TBSOTf) and 2,6-lutidine to form the compound of Formula 12.6:

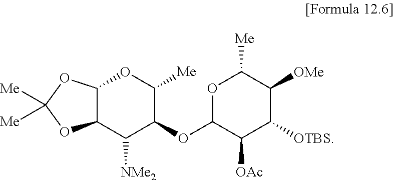

According to another aspect, the described invention provides a method for the synthesis of a compound of Formula 13.8:

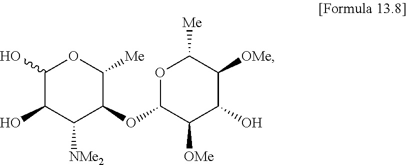 [SMILES: CO[C@H]1[C@H](O[C@H]2[C@H](N(C)C)[C@@H](O)C(O)O[C@@H]2C)O[C@H](C)[C@@H](OC)[C@@H]1O]

wherein the compound of Formula 13.8 matches the degraded sugar fragment from natural mangrolide A, comprising, in order: (a) reacting a compound of Formula 10.3:

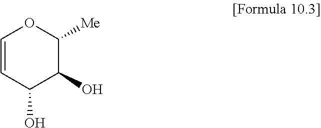

in the presence of acetylchloride (AcCl), pyridine (Py) and dichloromethane (CH₂Cl₂) to form a compound of Formula 13.1:

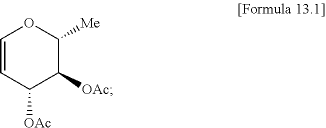 [SMILES: CC(=O)O[C@@H]1[C@@H](C)OC=C[C@H]1OC(C)=O]

(b) reacting the compound of Formula 13.1 in the presence of benzyl alcohol (BnOH), boron trifluoride etherate (BF3.OEt2) at 0° C. to form a compound of Formula 13.2:

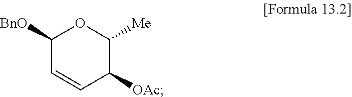 [SMILES: CC(=O)O[C@H]1C=C[C@@H](OCc2ccccc2)O[C@@H]1C]

(c) reacting the compound of Formula 13.2 in the presence of potassium carbonate (K2CO3) and methanol (MeOH) to form a compound of Formula 13.3:

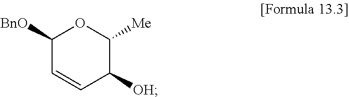 [SMILES: C[C@H]1O[C@H](OCc2ccccc2)C=C[C@@H]1O]

(d) reacting the compound of Formula 13.3 in the presence of meta-clhoroperoxybenzoic acid (mCPBA), sodium bicarbonate (NaHCO3), and dichlromethane (CH₂Cl₂) to form a compound of Formula 13.4:

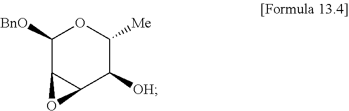 [SMILES: C[C@H]1O[C@H](OCc2ccccc2)[C@@H]2O[C@@H]2[C@@H]1O]

(e) reacting the compound of Formula 13.4 with a compound of Formula 10.9:

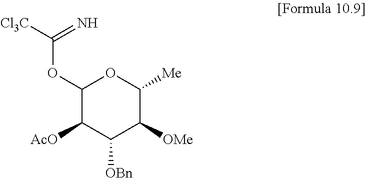

in the presence of boron trifluoride etherate, and dichloromethane at −15 C to form a compound of Formula 13.5:

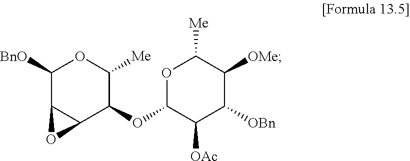

(f) reacting the compound of formula 13.5 in the presence of potassium hydroxide (KOH),

DMSO then methyl iodide (MEI) to form a compound of Formula 13.6:

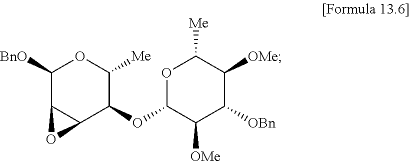

(g) reacting the compound of formula 13.6 in the presence of dimethylamine (Me2NH (aq)) and acetonitrile (MeCN) at 70° C. to form the compound of Formula 13.7:

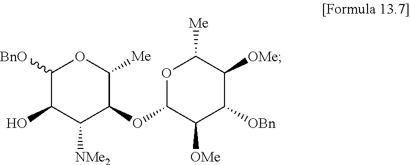 [SMILES: CO[C@H]1[C@H](O[C@H]2[C@H](N(C)C)[C@@H](O)C(OCc3ccccc3)O[C@@H]2C)O[C@H](C)[C@@H](OC)[C@@H]1OCc1ccccc1]

and (h) reacting the compound of Formula 13.7 in the presence of palladium on carbon (Pd/C), hydrogen gas (H₂), 1N HCl and ethanol (EtOH) to form the compound of Formula 13.8:

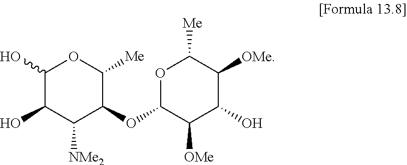

According to another aspect, the described invention provides a method for the synthesis of a fully synthetic, biologically active mangrolide A of Formula 14.5:

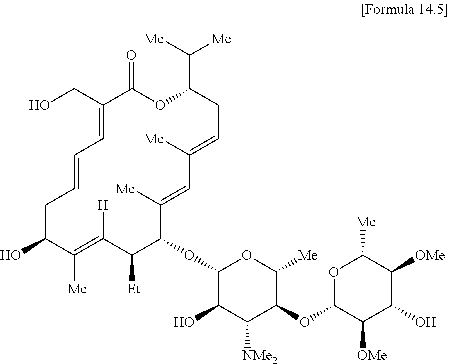 [SMILES: CC[C@H]1/C=C(\C)[C@@H](O)C/C=C/C=C(\CO)C(=O)O[C@H](C(C)C)C/C=C(C)/C=C(\C)[C@@H]1O[C@@H]1O[C@H](C)[C@@H](O[C@@H]2O[C@H](C)[C@@H](OC)[C@H](O)[C@H]2OC)[C@H](N(C)C)[C@H]1O]

comprising, in order: (a) reacting a compound of Formula 13.8:

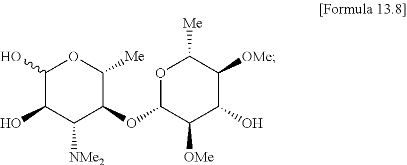 [SMILES: CO[C@H]1[C@H](O[C@H]2[C@H](N(C)C)[C@@H](O)C(O)O[C@@H]2C)O[C@H](C)[C@@H](OC)[C@@H]1O]

in the presence of acetic anhydride (Ac₂O), pyridine (Py), 4-dimethylaminopyridine (DMAP), and dichloromethane (CH₂Cl₂) to form a compound of Formula 14.1:

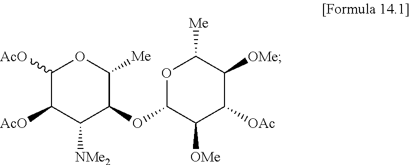

(b) reacting the compound of Formula 14.1 in the presence of thiophenol (PhSH) and Boron trifluoride diethyl etherate (BF₃OEt₂) to form a compound of Formula 14.2:

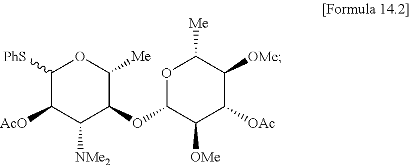

(c) reacting the compound of Formuula 14.2 and a compound of Formula 8.5:

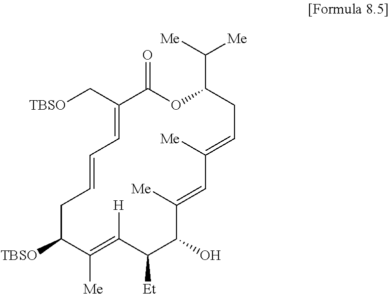 [SMILES: CC[C@H]1/C=C(\C)[C@@H](O[Si](C)(C)C(C)(C)C)C/C=C/C=C(\CO[Si](C)(C)C(C)(C)C)C(=O)O[C@H](C(C)C)C/C=C(C)/C=C(\C)[C@@H]1O]

in the presence of n-iodosuccinimide (NIS) and trimethylsilyl trifluoromethanesulfonate (TMSOf) to form a compound of Formula 14.3:

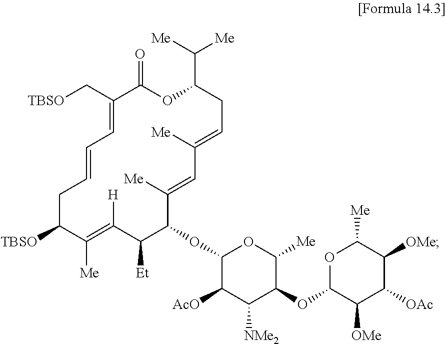

(d) reacting the compound of Formula 14.3 in the presence of potassium carbonate (K₂CO₃) and methanol (MeOH) to form a compound of Formula 14.4:

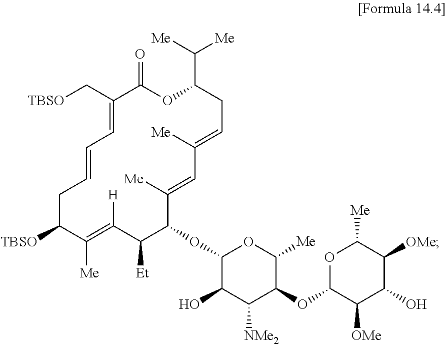

and (e) reacting the compound of Formula 14.4 in the presence of hydrofluoric acid (HF), pyridine (Py) and tetrahydrofuran (THF) to form the compound of Formula 14.5:

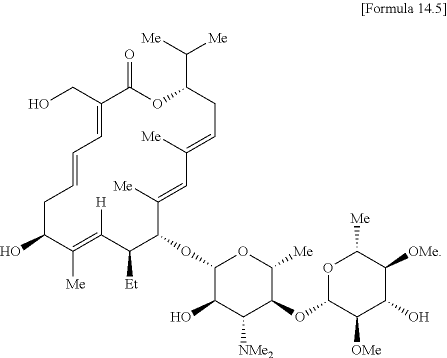

### BRIEF DESCRIPTION OF THE DRAWINGS

FIG. 1A shows proton NMR spectrum of synthetic C8-C9 Z isomer of mangrolide A aglycon methylether (possible structure A).

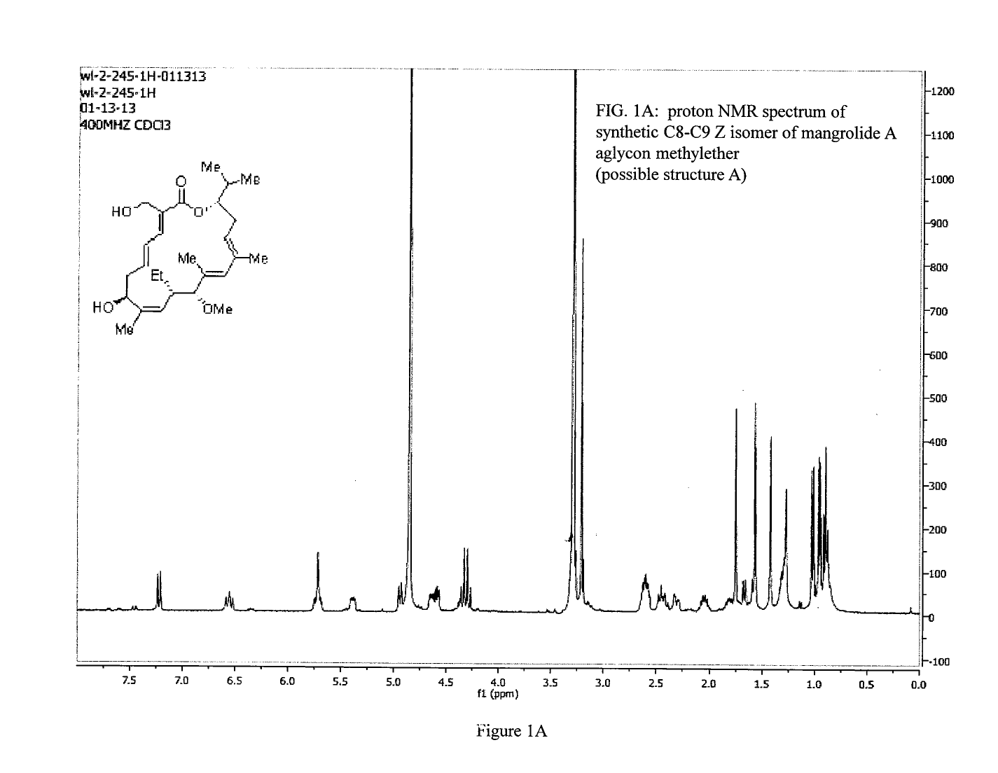

FIG. 1B shows carbon-13 NMR spectrum of synthetic C8-C9 Z-somer of mangrolide A aglycon methylether (possible structure A).

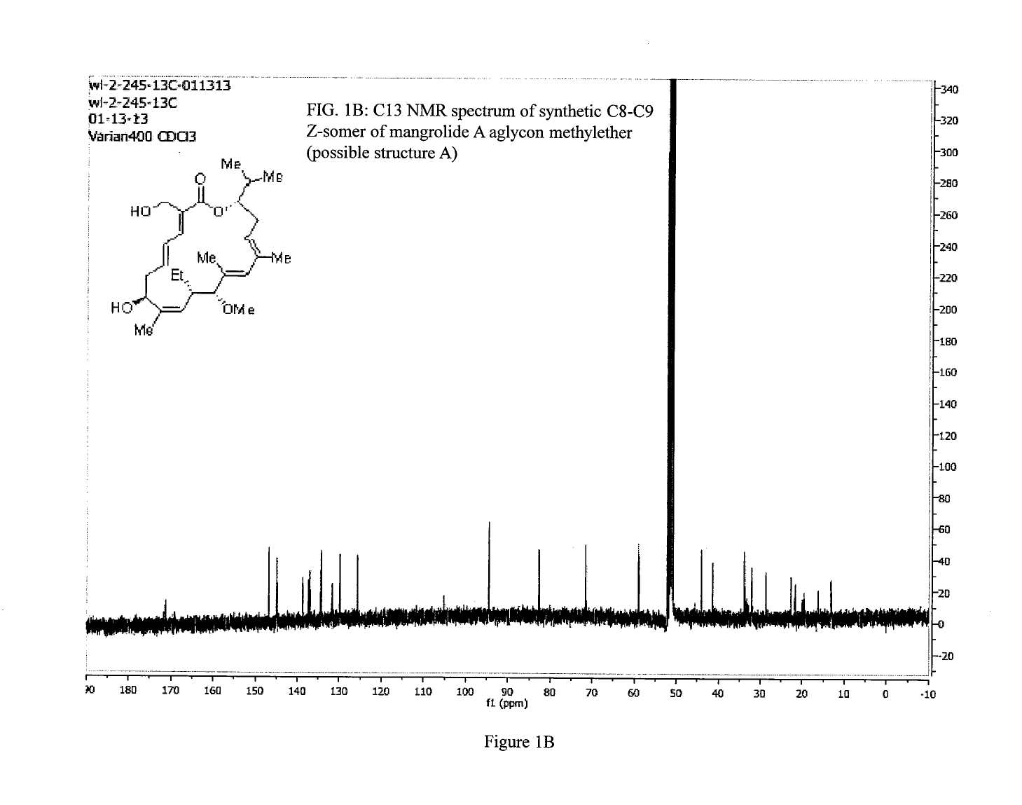

FIG. 2 shows proton NMR spectra of mangrolide aglycon methyl ether possible structcure B, which did not match the corresponding spectra obtained from degradation of natural mangrolide A.

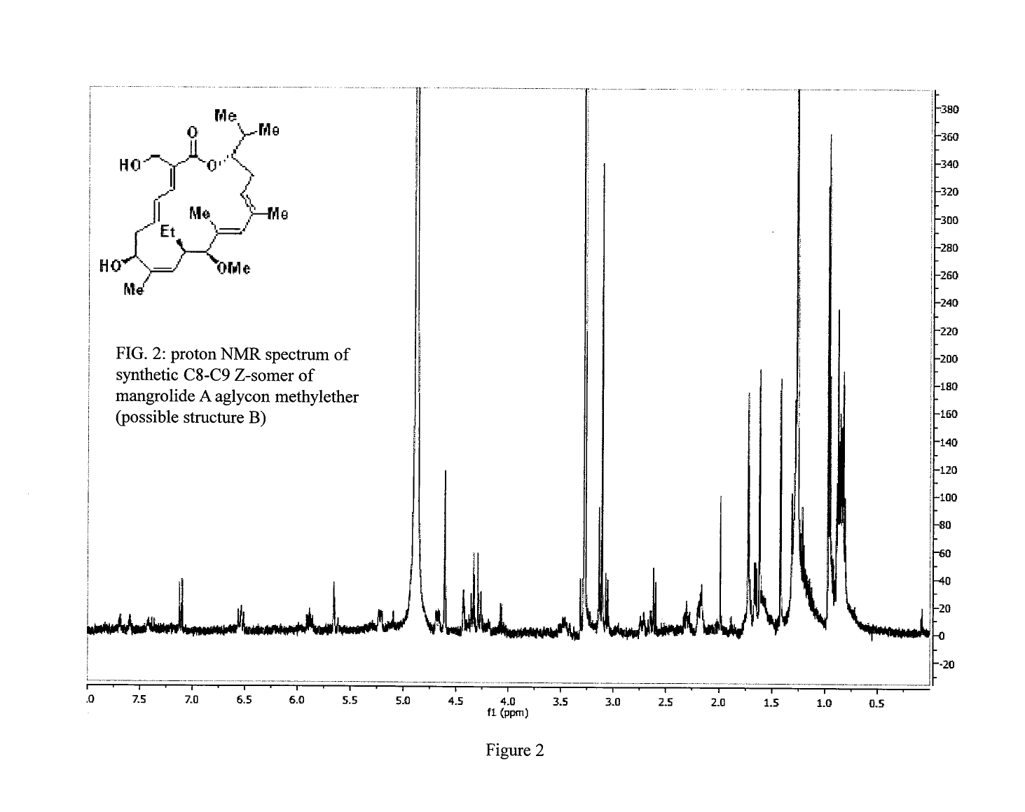

FIG. 3A shows carbon-13 NMR structure of synthetic C8-C9 E-isomer of mangrolide A aglycon methylether (possible structure C that matches natural mangrolide A aglycon methyl ether).

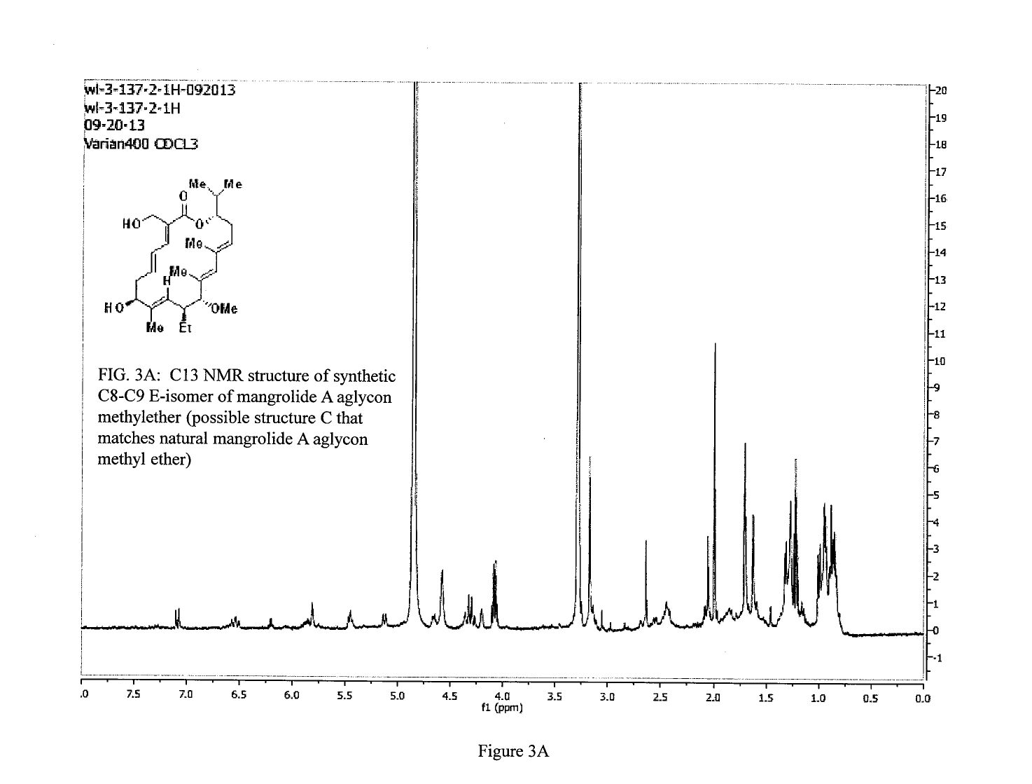

FIG. 3B shows proton NMR structure of synthetic C8-C9 E-isomer of mangrolide A aglycon methylether (possible structure C that matches natural mangrolide A aglycon methyl ether).

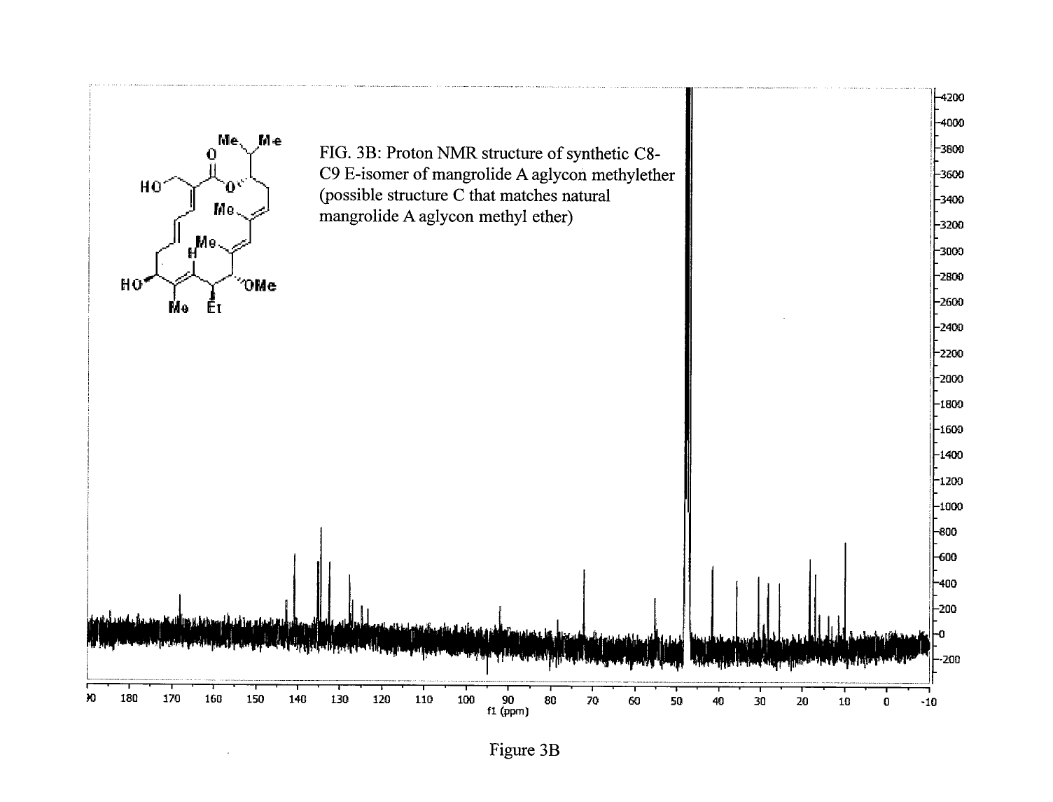

FIG. 4A shows proton NMR structure of natural mangrolide A aglycon methyl ether (obtained from degradation of natural mangrolide A).

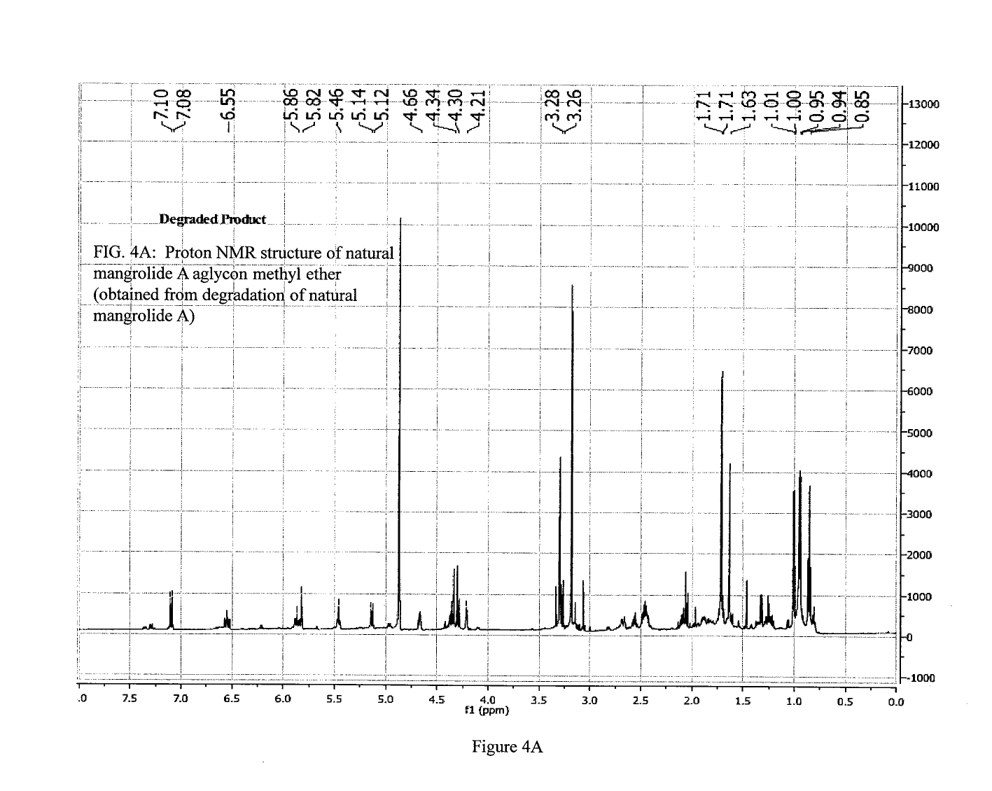

FIG. 4B shows carbon-13 NMR structure of natural mangrolide A aglycon methyl ether (obtained from degradation of natural mangrolide A).

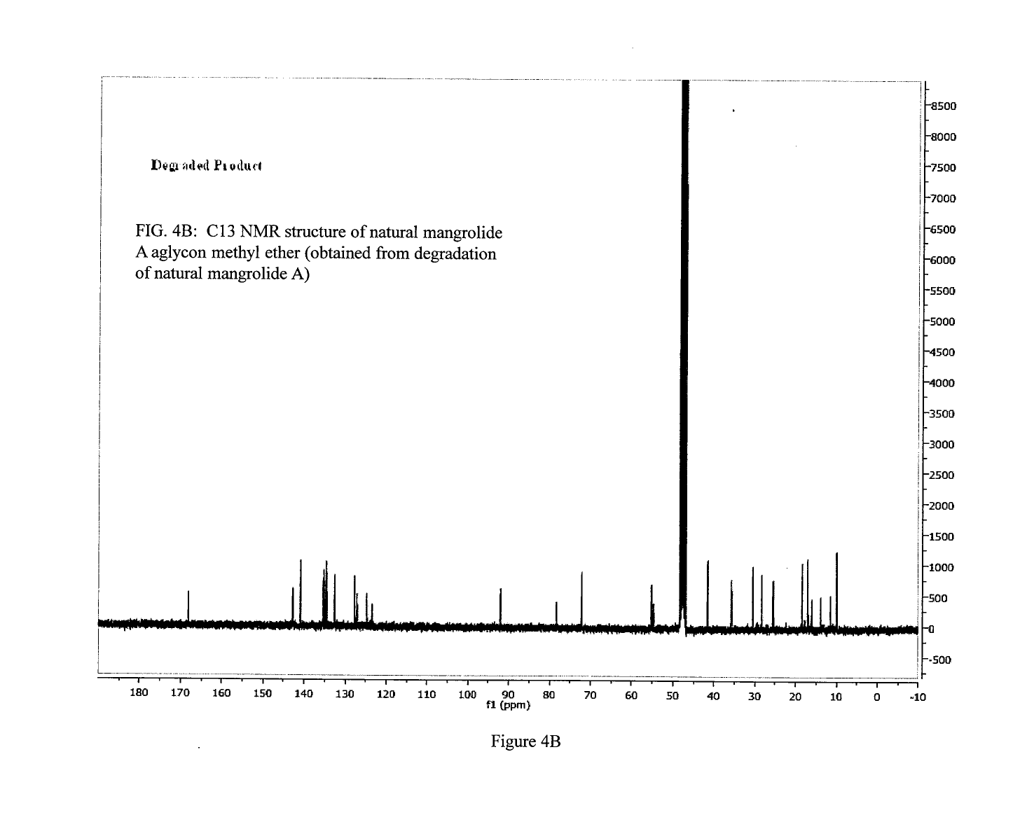

FIG. 4C shows proton NMR spectrum of synthetic mangrolide A disaccharide (matches natural disaccharide obtained from Mangrolide A degradation).

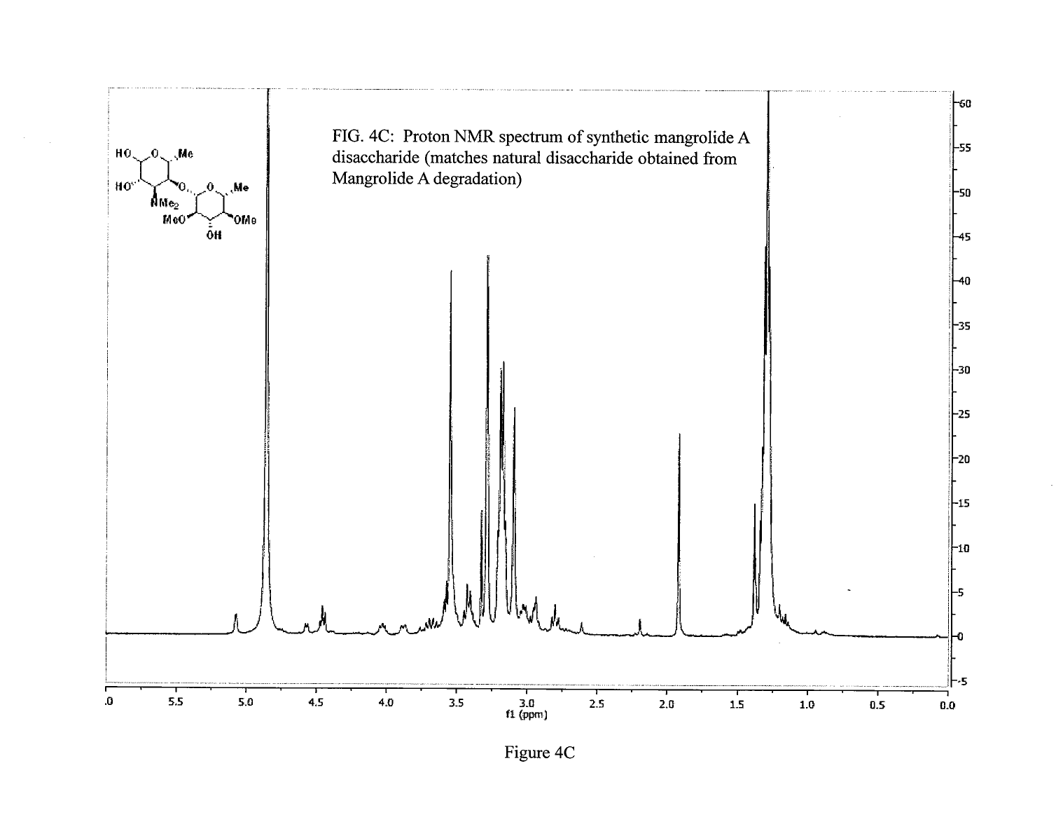

FIG. 4D shows proton NMR spectrum of natural mangrolide A disaccharide.

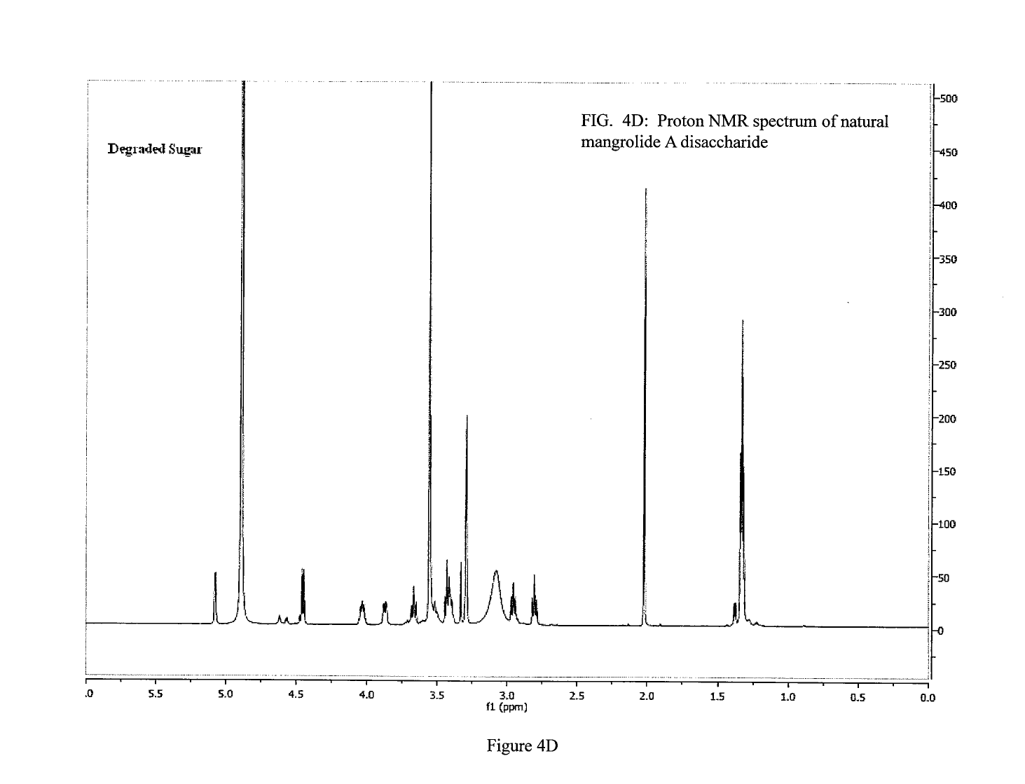

FIG. 5 shows the mean inhibitory concentration (MIC) of mangrolide A and an analog, mangrolide A O-methyl ether. (A) shows the structures of mangrolide A and a mangrolide A derivative, mangrolide A O-methyl ether. (B) Gramnegative and Gram-positive microbial pathogens were exposed to either mangrolide A or mangrolide A O-methyl ether. Antibiotic activity is measured in MIC μg/mL.

FIG. 6 is a plot of relative incorporation of 14C-phenylalanine or 3H-leucine versus concentration of mangrolide A, during protein synthesis, using the poly (UUU) RNA template [5′-GCGGCAAGGAGGUAAUAAUG (UUU)₁₂UAAGCAGG-3′ (SEQ ID NO: 1), 14C-phenylalanine, and 3H-lleucine and an i S30 extract from E. coli. It shows that mangrolide induces misincorporation of amino acids.

FIG. 7 shows that a 16S ribosome mutant is resistant to mangrolide. (A) An illustration depicting an A1408G (S30) mutation. (B) Is a plot of relative incorporation of a radiolabeled amino acid vs. mangrolide concentration. The results show that an A1408G (S30) mutation permits incorporation of 14C-Phe and 3H-Leu in the mutant comparable to that of wildtype. (C) Results of streaking wildtype or A1408G mutant bacteria on agar plates with or without mangrolide. The A1408G mutant strain was able to grow on plates with mangrolide, unlike the wildtype.

FIG. 8 is a plot showing radiolabeled amino acid incorporation during protein synthesis in S30 extracts of E. coli with or without neomycin phosphotransferase-1 (NPT) using the poly (UUU) RNA template [5′-GCGGCAAGGAGGUAAUAAUG (UUU)₁₂UAAGCAGG-3′(SEQ ID NO: 1), 14C-phenylalanine, and 3H-leucine. It shows that mangrolide is inactivated by neomycin phosphotransferase-1 (NPT).

### DETAILED DESCRIPTION

The described invention can be better understood from the following description of exemplary embodiments, taken in conjunction with the accompanying figures and drawings.

### Definitions

Various terms used throughout this specification shall have the definitions as set out herein.

The terms “administering” or “administration” as used herein are used interchangeably to mean the giving or applying of a substance and include in vivo administration, as well as administration directly to tissue ex vivo. Generally, compositions may be administered systemically either orally, buccally, parenterally, topically, by inhalation or insufflation (i.e., through the mouth or through the nose), or rectally in dosage unit formulations containing conventional nontoxic pharmaceutically acceptable carriers, adjuvants, and vehicles as desired, or may be locally administered by means such as, but not limited to, injection, implantation, grafting, topical application, or parenterally.

The terms “analog” and “derivative” are used interchangeably to mean a compound produced from another compound of similar structure in one or more steps. A “derivative” or “analog” of a compound retains at least a degree of the desired function of the reference compound. Accordingly, an alternate term for “derivative” may be “functional derivative.” Derivatives can include chemical modifications, such as akylation, acylation, carbamylation, iodination or any modification that derivatizes the compound. Such derivatized molecules include, for example, those molecules in which free amino groups have been derivatized to form amine hydrochlorides, p-toluene sulfonyl groups, carbobenzoxy groups, t-butyloxycarbonyl groups, chloroacetyl groups or formal groups. Free carboxyl groups can be derivatized to form salts, esters, amides, or hydrazides. Free hydroxyl groups can be derivatized to form O-acyl or O-alkyl derivatives.

The term “aerobe” as used herein refers to a bacterium that can survive and grow in an oxygenated environment.

The term “anaerobe” as used herein refers to a bacterium which can grow in the absence of oxygen.

The term “antibiotic” is a substance produced by or derived from certain fungi, bacteria, other organisms or produced synthetically that can destroy or inhibit the growth of other microorganisms. Antibiotics are widely used in the prevention and treatment of infectious diseases.

The term “Area Under the Curve (AUC)” as used herein refers to an index used to measure the linearity of a drug's dose-response relationship obtained from a plot of serum concentration versus time. A linear dose-response relationship makes it easy to predict serum concentration as a function of dose.

The term “β-Lactam” as used herein refers to a class of antibiotics, including penicillins, cephalosporins, and carbapenems, which irreversibly inactivate DD-transpeptidases involved in the cross-linking of bacterial peptidoglycan, often causing cell lysis.

The term “bacterial ribosome” refers to a cytoplasmic nucleoprotein particle whose main function is to serve as the site of mRNA translation and protein synthesis. The bacterial ribosome consists of two subunits denoted 30S (small subunit) and 50S (large subunit). When the small subunit and large subunit are joined, the ribosome has a sedimentation coefficient of 70S as opposed to the sedimentation coeffeicient of mammalian ribosomes of 80S.

The term “bacteriocidal effect” or “bactericidal effect” refers to a drug effect in which bacteria are killed.

The term “bacteriostatic effect” refers to a drug effect in which bacterial multiplication is prevented or inhibited even though the microorganism remains alive and can metabolize/use nutrients.

The term “bacilli” or “bacillus” refers to cylindrical (rod-shaped) bacterial organism(s).

The term “bioactivity/bioavailability” as used herein refers to the extent to which a drug is dissolved, absorbed, and then distributed to site(s) where it is active.

The term “clinical isolate” as used herein refers to bacterium (bacteria) isolated from a patient.

The term “Cmax” as used herein refers to Maximum (peak) serum concentration. Cmax must exceed the in vitro MIC to effectively combat a bacterial infection.

The term “coccus,” plural “cocci,” as used herein means spherical-shaped bacterial organism(s). Many species of bacteria have characteristic arrangements that are useful in identification. Pairs of cocci are diplococci; rows or chains of such cells are called streptococci; grapelike clusters of cells, staphylococci; packets of eight or more cells, sarcinae; and groups of four cells in a square arrangement, tetrads. These characteristic groupings occur as a result of variations in the reproduction process in bacteria.

The term “colony” as used herein means at the proper concentration/dilution, a bacterium and the progeny of that bacterium visible with the naked eye that have grown on solid agar in flat plastic plates.

The term “colony forming unit” or “CFU” as used herein, at the proper concentration, is a single bacterium.

The term “condition”, as used herein, refers to a variety of health states and is meant to include disorders or diseases caused by any underlying mechanism or injury.

The term “cross-resistance” as used herein refers to resistance against one drug that results in resistance against another drug.

The term “culture” as used herein refers to a population of bacteria grown in liquid nutrient medium.

The term “disease” or “disorder,” as used herein, refers to an impairment of health or a condition of abnormal functioning.

The term “drug” as used herein refers to a therapeutic agent or any substance used in the prevention, diagnosis, alleviation, treatment, or cure of disease.

The term “ED₉₀₋₁₀₀” as used herein refers to a therapeutic or effective dose, i.e., the mg/kg drug required to achieve an infection cure rate of 90-100%.

The term “Gram stain test” as used herein refers to a staining procedure which differentiates bacteria into two groups (Gram positive and Gram negative) on the basis of differences in their cell-wall structure. In brief, bacteria are heat-fixed to slides, stained with a purple dye (crystal violet), which is then fixed, washed with alcohol or acetone, and counterstained with a red dye. Gram positive organisms retain the purple dye because their cell wall acts as a permeability barrier to elution of the initial staining complex. Gram negative organisms are stained red because their cell walls allow the organic solvent to decolorize them.

The term “growth curve” as used herein refers to bacterial growth, which is characteristically exponential. However, an exponentially growing culture, referred to as a culture in “log phase,” eventually slows down and stops growing (“stationary phase”). In this stationary phase, the cells become smaller as a result of dividing faster than they grow. Stationary phase cells transferred to fresh medium exhibit a “lag phase,” which is a period of time between the introduction of a microorganism into a culture medium and the time it begins to increase in number exponentially. Cell number lags more than cell mass because the small stationary-phase cells increase in size before they begin to divide.

The term “Inhibitory Quotient (I.Q.)” as used herein refers to the ratio between serum peak drug concentration and MIC calculated from one point in time.

The term “injury,” as used herein, refers to damage or harm to a structure or function of the body caused by an outside agent or force, which may be physical or chemical.

The term “killing curve,” as used herein, refers to a plot of number of bacterium (y-axis) against time (x-axis) in the presence of a particular drug concentration(s). A killing curve represents the killing rate and the efficiency of a drug's interaction with the test organism(s).

The term “Mangrolide A” refers to an antibiotic, either produced naturally by marine actinomycetes, derived from a mangrove swamp sediment collected at Sweetings Cay, Bahamas or synthesized synthetically. The marin actinomycetes organism has about 97% similarity to Actinoalloteichus spitiensis (16S rRNA). Natural mangrolide A exhibits bactericidal/bacteriostatic activity against Pseudomonas aeruginosa and Burkholderia.

The term “maximum tolerated dose” or MTD as used herein refers to the highest dose of a pharmacological treatment (e.g., Magrolide A) that will produce the desired effect without unacceptable toxicity.

The term “MBC” as used herein refers to the lowest concentration of a drug that will kill 99.9% of the organisms. MBC is referred to in the literature as either most (or minimal) bactericidal concentration.

The term “MBC90” as used herein refers to the lowest concentration of a drug that will kill 90% of bacteria in a culture.

The term “mechanism of action” or “MOA” is the means by which a pharmacologically active substance produces an effect on a living organism or in a biochemical system. The MOA is usually considered to include an identification of the specific molecular targets to which a pharmacologically active substance binds or whose biochemical action it influences; and a general recognition of the broad biochemical pathways (such as DNA synthesis, protein synthesis) which are inhibited or affected by a substance.

The term “minimal inhibitory concentration” (MIC) refers to the lowest concentration of an antimicrobial agent that, under defined test conditions, inhibits the visible growth of the bacterium being investigated. MIC values are used to determine susceptibilities of bacteria to drugs and also to evaluate the activity of new antimicrobial agents. (Wiegand et al., Nature Protocols, 2008, 3:163-175).

The term “MIC50” refers to the lowest concentration of a drug that will inhibit the growth of 50% of bacteria in a culture.

The term “MIC90” refers to the lowest concentration of a drug that will inhibit the growth of 90% of bacteria in a culture.

The term “modify” as used herein means to change, vary, adjust, temper, alter, affect or regulate to a certain measure or proportion in one or more particulars.

The term “modifying agent” as used herein refers to a substance, composition, extract, botanical ingredient, botanical extract, botanical constituent, therapeutic component, active constituent, therapeutic agent, drug, metabolite, active agent, protein, non-therapeutic component, non-active constituent, non-therapeutic agent, or non-active agent that reduces, lessens in degree or extent, or moderates the form, symptoms, signs, qualities, character or properties of a condition, state, disorder, disease, symptom or syndrome.

The term “modulate” as used herein means to regulate, alter, adapt, or adjust to a certain measure or proportion.

The term “multi-drug resistant” refers to organisms that, have become resistant to drugs used to treat them, i.e., a particular drug is no longer able to kill or control growth of the bacteria. Bacteria that resist treatment with more than one antibiotic are multi-drug resistant organisms. Multi-drug resistant organisms are found mainly in hospitals and long-term care facilities. Other terms to describe this occurrence include antibiotic resistance, antibacterial resistance, and antimicrobial resistance. Some common examples of these multi-drug resistant organisms are healthcare associated methicillin-resistant Staphylococcus aureus (MRSA), vancomycin-resistant enterococci (VRE), extended-spectrum beta lactamsases (ESBL), and penicillin-resistant Streptococcus pneumoniae (PRSP). Multi-drug resistant organisms can cause infections in almost any part of the body, including the skin, lungs, urinary tract, bloodstream, and wounds.

As used herein, the term “mutation” refers to a change of the DNA sequence within a gene or chromosome of an organism resulting in the creation of a new character or trait not found in the parental type, or the process by which such a change occurs in a chromosome, either through an alteration in the nucleotide sequence of the DNA coding for a gene or through a change in the physical arrangement of a chromosome. Three mechanisms of mutation include substitution (exchange of one base pair for another), addition (the insertion of one or more bases into a sequence), and deletion (loss of one or more base pairs).

The terms “mutants” and “variants” are used interchangeably herein to refer to nucleotide sequences with substantial identity to a reference nucleotide sequence. The differences in the sequences may be the result of changes, either naturally or by design, in sequence or structure. Natural changes may arise during the course of normal replication or duplication in nature of the particular nucleic acid sequence. Designed changes may be specifically introduced into the sequence for specific purposes. Such specific changes may be made in vitro using a variety of techniques.

The term “penetration” as used herein refers to ability of a substance to enter the interior of, pass into or through a membrane into a cell.

The term “pharmaceutical composition” as used herein refers to a preparation comprising a pharmaceutical product, drug, metabolite, or active ingredient.

The term “pharmacokinetics” as used herein refers to the use of pharmacological parameters that together relate to a drug's bioavailability to characterize a drug.

The term “post antibiotic effect” as used herein refers to the delayed regrowth of surviving bacteria following transient exposure to an antibiotic. Post antibiotic effect is bacterial species-specific and is affected by antibiotic concentration, length of exposure, growth conditions, and the bacterial inoculum.

The term “potency” as used herein refers to drug potency within whole bacterial cells. A drug's potency is a complex sum of its ability to penetrate into the cell's interior (where the drug target(s) are located), how tightly the drug binds to the target(s), the drug's ability to interfere with the cell's metabolic function after binding to the target(s), and the extent to which this interference is lethal to the bacterial cell.

The term “prodrug” as used herein refers to a compound that, on administration, must be chemically converted in the body by normal metabolic processes from an inactive form into an active form.

The term “reduce” or “reducing” as used herein refers to limiting occurrence of a disorder in individuals at risk of developing the disorder.

The term “renal clearance” as used herein refers to the amount of drug dose excreted in urine 24 h after administered dose.

The term “ribosomal RNA” (rRNA) as used herein refers to ribonucleic acid (RNA) that is synthesized in the nucleolus from genes that encode rRNA. Ribosomal RNA is a structural component of a ribosome. The rRNAs form extensive secondary structures and play an active role in recognizing conserved portions of mRNAs and tRNAs. Each subunit of the ribosome is about two-thirds RNA and one-third protein. The ribosomal proteins are tightly associated with each other and with the RNA. The small bacterial ribosomal subunit contains one each of 21 different proteins, named S1 to S21 according to their positions in gel electrophoresis. The large bacterial ribosomal subunit contains proteins L1 to L34. (Davis et al., Microbiology, (1980) 3^{rd} Ed., Harper & Row Publishers, at 235)

The term “ring compound” as used herein refers to a compound in which carbon atoms are arranged to form rings.

The term “serum bactericidal activity” (SBA) as used herein refers to an in vitro test of drug efficacy that measures the greatest dilution activity of a serum sample (obtained from a patient or subject after that subject has received an antibiotic) that inhibits the growth of a given microorganism (serum inhibitory titer) or kills more than 99.9% of a defined inoculum (serum bactericidal titer).

The term “serum half-life” as used herein refers to the time within which the concentration of a drug in serum is reduced by half.

The term “stationary culture” as used herein refers to a bacterial culture that is not multiplying in number but, however, does utilize nutrients at a slow rate.

The term “Staphylococci” or “Staphylococcus” as used herein refers to spherical organism(s) which appear in clusters like a bunch of grapes.

The term “Streptococci” or “Streptococcus” as used herein refers to spherical organism(s) which appear in chains.

The terms “subject” or “individual” or “patient” are used interchangeably to refer to a member of an animal species of mammalian origin, including humans.

As used herein, the term “substantially pure” refers to purity of at least 75%, at least 80%, at least 85%, at least 90%, at least 95% or at least 99% pure as determined by an analytical protocol.

The term “symptom” as used herein refers to a phenomenon that arises from and accompanies a particular disease or disorder and serves as an indication of it.

The term “syndrome,” as used herein, refers to a pattern of symptoms indicative of some disease or condition.

The term “synergy” as used herein refers to a synergistic effect of one antibiotic with other antibiotics. Synergy is usually defined by a reduction in the MIC or MBC of at least four-fold or a fractional inhibitory index of 0.5 or less.

The term “therapeutic agent” as used herein refers to a drug, molecule, nucleic acid, protein, metabolite, composition or other substance that provides a therapeutic effect. The term “active” as used herein refers to the ingredient, component or constituent of the compositions of the described invention responsible for the intended therapeutic effect. The terms “therapeutic agent” and “active agent” are used interchangeably herein. The active agent may be, for example, but not limited to, at least one of a compound of formula I, or a pharmaceutically acceptable salt thereof.

The term “therapeutic component” as used herein refers to a therapeutically effective dosage (i.e., dose and frequency of administration) that eliminates, reduces, or prevents the progression of a particular disease manifestation in a percentage of a population. An example of a commonly used therapeutic component is the ED50, which describes the dose in a particular dosage that is therapeutically effective for a particular disease manifestation in 50% of a population.

The term “therapeutic effect” as used herein refers to a consequence of treatment, the results of which are judged to be desirable and beneficial. A therapeutic effect may include, directly or indirectly, the arrest, reduction, or elimination of a disease manifestation. A therapeutic effect may also include, directly or indirectly, the arrest reduction or elimination of the progression of a disease manifestation.

The term “Tmax” as used herein refers to a period of time for serum concentration to reach its peak, i.e., time until Cmax is reached.

As used herein, the term “topical” refers to administration of a composition at, or immediately beneath, the point of application. The phrase “topically applying” describes application onto one or more surfaces(s) including epithelial surfaces. Topical administration, generally provides a local rather than a systemic effect.

As used herein the term “treating” includes abrogating, substantially inhibiting, slowing or reversing the progression of a condition, substantially ameliorating clinical symptoms of a condition, or substantially preventing the appearance of clinical symptoms of a condition. Treating further refers to accomplishing one or more of the following: (a) reducing the severity of the disorder; (b) limiting development of symptoms characteristic of the disorder(s) being treated; (c) limiting worsening of symptoms characteristic of the disorder(s) being treated; (d) limiting recurrence of the disorder(s) in patients that have previously had the disorder(s); and (e) limiting recurrence of symptoms in patients that were previously asymptomatic for the disorder(s). The term “condition” as used herein refers to a variety of health states and is meant to include disorders or diseases caused by any underlying mechanism or disorder in which bacteria infect a subject. A subject in need thereof is a patient having, or at risk of having a disorder related to bacterial infection.

As used herein, the term “Volume of Distribution” reflects a drug's distribution in the body's cells, organs, and tissue. A measurement showing that a drug's volume of distribution exceeds the volume of normal body water (0.6 l/kg) indicates that that drug is being concentrated in tissues.

### Chemical Substituents

The term “Aliphatic” as used herein, denotes a straight- or branched-chain arrangement of constituent carbon atoms, including, but not limited to paraffins (alkanes), which are saturated, olefins (alkenes or alkadienes), which are unsaturated, and acetylenes (alkynes), which contain a triple bond. In complex structures, the chains may be branched or cross-linked.

The term “lower” as used herein refers to a group having between one and six carbons.

As used herein, the term “alkyl” refers to a straight or branched chain hydrocarbon having from 1 to 25 carbon atoms, optionally substituted with substituents such as, but not limited to, nitro, cyano, halogen, perfluoroalkyl, aryl, heteroaryl, heterocyclyl, cycloalkyl, lower alkyl, lower alkoxy, lower alkylsulfanyl, lower alkylsulfenyl, lower alkylsulfonyl, oxo, hydroxy, mercapto, amino optionally substituted by alkyl or aryl or heteroaryl or heterocyclyl or cycloalkyl, aminocarbonyl (—NRC(O)R) optionally substituted by alkyl or aryl or heteroaryl or heterocyclyl or cycloalkyl, carboxy, acyl, acyloxy, alkoxycarbonyl, aryloxy, heteroaryloxy, heterocyclyloxy, aroyloxy, heteroaroyloxy, heterocycloyloxy, carbamoyl optionally substituted by alkyl or cycloalkyl or aryl or heteroaryl or heterocyclyl, aminosulfonyl optionally substituted by alkyl or cycloalkyl or aryl or heteroaryl or heterocyclyl, silyloxy optionally substituted by alkyl or aryl, silyl optionally substituted by alkoxy or alkyl or aryl, multiple degrees of substitution being allowed. Such an “alkyl” group may contain one or more O, S, S(O), or S(O)₂ atoms. Examples of “alkyl” as used herein include, but are not limited to, methyl, ethyl, propyl, decyl, undecyl, octadecyl, nonadecyl, eicosyl, heneicosyl, decosyl, tricosyl, tetracosyl, and pentacosyl, n-butyl, t-butyl, n-pentyl, isobutyl, and isopropyl, and the like.

The term “alkylene” as used herein refers to a straight or branched chain divalent hydrocarbon radical having from one to 25 carbon atoms, optionally substituted with substituents such as, but not limited to, nitro, cyano, halogen, perfluoroalkyl, aryl, heteroaryl, heterocyclyl, cycloalkyl, lower alkyl, lower alkoxy, lower alkylsulfanyl, lower alkylsulfenyl, lower alkylsulfonyl, oxo, hydroxy, mercapto, amino optionally substituted by alkyl or aryl or heteroaryl or heterocyclyl or cycloalkyl, aminocarbonyl (—NRC(O)R) optionally substituted by alkyl or aryl or heteroaryl or heterocyclyl or cycloalkyl, carboxy, acyl, acyloxy, alkoxycarbonyl, aryloxy, heteroaryloxy, heterocyclyloxy, aroyloxy, heteroaroyloxy, heterocycloyloxy, carbamoyl optionally substituted by alkyl or cycloalkyl or aryl or heteroaryl or heterocyclyl, aminosulfonyl optionally substituted by alkyl or cycloalkyl or aryl or heteroaryl or heterocyclyl, silyloxy optionally substituted by alkyl or aryl, silyl optionally substituted by alkoxy or alkyl or aryl, multiple degrees of substitution being allowed. Such an “alkylene” group may contain one or more O, S, S(O), or S(O)₂ atoms. Examples of “alkylene” as used herein include, but are not limited to, methylene, ethylene, and the like.

The term “Alkenyl,” as used herein, denotes a monovalent, straight (unbranched) or branched hydrocarbon chain having one or more double bonds therein where the double bond can be unconjugated or conjugated to another unsaturated group (e.g., a polyunsaturated alkenyl) and can be unsubstituted or substituted, with multiple degrees of substitution being allowed. It may be optionally substituted with substituents such as, but not limited to, nitro, cyano, halogen, perfluoroalkyl, aryl, heteroaryl, heterocyclyl, cycloalkyl, lower alkyl, lower alkoxy, lower alkylsulfanyl, lower alkylsulfenyl, lower alkylsulfonyl, oxo, hydroxy, mercapto, amino optionally substituted by alkyl or aryl or heteroaryl or heterocyclyl or cycloalkyl, aminocarbonyl (—NRC(O)R) optionally substituted by alkyl or aryl or heteroaryl or heterocyclyl or cycloalkyl, carboxy, acyl, acyloxy, alkoxycarbonyl, aryloxy, heteroaryloxy, heterocyclyloxy, aroyloxy, heteroaroyloxy, heterocycloyloxy, carbamoyl optionally substituted by alkyl or cycloalkyl or aryl or heteroaryl or heterocyclyl, aminosulfonyl optionally substituted by alkyl or cycloalkyl or aryl or heteroaryl or heterocyclyl, silyloxy optionally substituted by alkyl or aryl, silyl optionally substituted by alkoxy or alkyl or aryl, multiple degrees of substitution being allowed. Such an “alkenyl” group may contain one or more O, S, S(O), or S(O)₂ atoms. For example, and without limitation, the alkenyl can be vinyl, allyl, butenyl, pentenyl, hexenyl, butadienyl, pentadienyl, hexadienyl, 2-ethylhexenyl, 2-propyl-2-butenyl, 4-(2-methyl-3-butene)-pentenyl, decenyl, undecenyl, dodecenyl, heptadecenyl, octadecenyl, nonadecenyl, eicosenyl, heneicosenyl, docosenyl, tricosenyl, tetracisenyl, pentacosenyl, phytyl, the branched chain isomers thereof, and polyunsaturated alkenes including octadec-9,12,-dienyl, octadec-9,12,15-trienyl, and eicos-5,8,11,14-tetraenyl.

As used herein, the term “alkenylene” refers to a straight or branched chain divalent hydrocarbon radical having from 2 to 25 carbon atoms and one or more carbon-carbon double bonds, optionally substituted with substituents such as, without limitation, nitro, cyano, halogen, perfluoroalkyl, aryl, heteroaryl, heterocyclyl, cycloalkyl, lower alkyl, lower alkoxy, lower alkylsulfanyl, lower alkylsulfenyl, lower alkylsulfonyl, oxo, hydroxy, mercapto, amino optionally substituted by alkyl or aryl or heteroaryl or heterocyclyl or cycloalkyl, aminocarbonyl (—NRC(O)R) optionally substituted by alkyl or aryl or heteroaryl or heterocyclyl or cycloalkyl, carboxy, acyl, acyloxy, alkoxycarbonyl, aryloxy, heteroaryloxy, heterocyclyloxy, aroyloxy, heteroaroyloxy, heterocycloyloxy, carbamoyl optionally substituted by alkyl or cycloalkyl or aryl or heteroaryl or heterocyclyl, aminosulfonyl optionally substituted by alkyl or cycloalkyl or aryl or heteroaryl or heterocyclyl, silyloxy optionally substituted by alkyl or aryl, silyl optionally substituted by alkoxy or alkyl or aryl, multiple degrees of substitution being allowed. Such an “alkenylene” group may contain one or more O, S, S(O), or S(O)₂ atoms. Examples of “alkenylene” as used herein include, but are not limited to, ethene-1,2-diyl, propene-1,3-diyl, methylene-1,1-diyl, and the like.

As used herein, the term “alkynyl” refers to a hydrocarbon radical having from 2 to 25 carbons and at least one carbon-carbon triple bond, optionally substituted with substituents such as, without limitation, nitro, cyano, halogen, perfluoroalkyl, aryl, heteroaryl, heterocyclyl, cycloalkyl, lower alkyl, lower alkoxy, lower alkylsulfanyl, lower alkylsulfenyl, lower alkylsulfonyl, oxo, hydroxy, mercapto, amino optionally substituted by alkyl or aryl or heteroaryl or heterocyclyl or cycloalkyl, aminocarbonyl (—NRC(O)R) optionally substituted by alkyl or aryl or heteroaryl or heterocyclyl or cycloalkyl, carboxy, acyl, acyloxy, alkoxycarbonyl, aryloxy, heteroaryloxy, heterocyclyloxy, aroyloxy, heteroaroyloxy, heterocycloyloxy, carbamoyl optionally substituted by alkyl or cycloalkyl or aryl or heteroaryl or heterocyclyl, aminosulfonyl optionally substituted by alkyl or cycloalkyl or aryl or heteroaryl or heterocyclyl, silyloxy optionally substituted by alkyl or aryl, silyl optionally substituted by alkoxy or alkyl or aryl, multiple degrees of substitution being allowed. Such an “alkynyl” group may contain one or more O, S, S(O), or S(O)₂ atoms.

As used herein, the term “alkynylene” refers to a straight or branched chain divalent hydrocarbon radical having from 2 to 25 carbon atoms and one or more carbon-carbon triple bonds, optionally substituted with substituents such as, without limitation, nitro, cyano, halogen, perfluoroalkyl, aryl, heteroaryl, heterocyclyl, cycloalkyl, lower alkyl, lower alkoxy, lower alkylsulfanyl, lower alkylsulfenyl, lower alkylsulfonyl, oxo, hydroxy, mercapto, amino optionally substituted by alkyl or aryl or heteroaryl or heterocyclyl or cycloalkyl, aminocarbonyl (—NRC(O)R) optionally substituted by alkyl or aryl or heteroaryl or heterocyclyl or cycloalkyl, carboxy, acyl, acyloxy, alkoxycarbonyl, aryloxy, heteroaryloxy, heterocyclyloxy, aroyloxy, heteroaroyloxy, heterocycloyloxy, carbamoyl optionally substituted by alkyl or cycloalkyl or aryl or heteroaryl or heterocyclyl, aminosulfonyl optionally substituted by alkyl or cycloalkyl or aryl or heteroaryl or heterocyclyl, silyloxy optionally substituted by alkyl or aryl, silyl optionally substituted by alkoxy or alkyl or aryl, multiple degrees of substitution being allowed. Such an “alkynylene” group may contain one or more O, S, S(O), or S(O)₂ atoms. Examples of “alkynylene” as used herein include, but are not limited to, ethyne-1,2-diyl, propyne-1,3-diyl, and the like.

The term “aryl” as used herein refers to a benzene ring or to an optionally substituted benzene ring system fused to one or more optionally substituted benzene rings, with multiple degrees of substitution being allowed. Substituents include, but are not limited to, nitro, cyano, halogen, perfluoroalkyl, aryl, heteroaryl, heterocyclyl, cycloalkyl, lower alkyl, lower alkoxy, lower alkylsulfanyl, lower alkylsulfenyl, lower alkylsulfonyl, oxo, hydroxy, mercapto, amino optionally substituted by alkyl or aryl or heteroaryl or heterocyclyl or cycloalkyl, aminocarbonyl (—NRC(O)R) optionally substituted by alkyl or aryl or heteroaryl or heterocyclyl or cycloalkyl, carboxy, acyl, acyloxy, alkoxycarbonyl, aryloxy, heteroaryloxy, heterocyclyloxy, aroyloxy, heteroaroyloxy, heterocycloyloxy, carbamoyl optionally substituted by alkyl or cycloalkyl or aryl or heteroaryl or heterocyclyl, aminosulfonyl optionally substituted by alkyl or cycloalkyl or aryl or heteroaryl or heterocyclyl, silyloxy optionally substituted by alkyl or aryl, silyl optionally substituted by alkoxy or alkyl or aryl, multiple degrees of substitution being allowed. Examples of aryl include, but are not limited to, phenyl, 2-napthyl, 1-naphthyl, 1-anthracenyl, and the like.

It should be understood that wherever the terms “alkyl” or “aryl” or either of their prefix roots appear in a name of a substituent, they are to be interpreted as including those limitations given above for alkyl and aryl. Designated numbers of carbon atoms (e.g. C₁₋₁₀) shall refer independently to the number of carbon atoms in an alkyl, alkenyl or alkynyl or cyclic alkyl moiety or to the alkyl portion of a larger substituent in which the term “alkyl” appears as its prefix root.

As used herein, the term “arylene” refers to a benzene ring diradical or to a benzene ring system diradical fused to one or more optionally substituted benzene rings, with multiple degrees of substitution being allowed. Substituents include, but are not limited to, nitro, cyano, halogen, perfluoroalkyl, aryl, heteroaryl, heterocyclyl, cycloalkyl, lower alkyl, lower alkoxy, lower alkylsulfanyl, lower alkylsulfenyl, lower alkylsulfonyl, oxo, hydroxy, mercapto, amino optionally substituted by alkyl or aryl or heteroaryl or heterocyclyl or cycloalkyl, aminocarbonyl (—NRC(O)R) optionally substituted by alkyl or aryl or heteroaryl or heterocyclyl or cycloalkyl, carboxy, acyl, acyloxy, alkoxycarbonyl, aryloxy, heteroaryloxy, heterocyclyloxy, aroyloxy, heteroaroyloxy, heterocycloyloxy, carbamoyl optionally substituted by alkyl or cycloalkyl or aryl or heteroaryl or heterocyclyl, aminosulfonyl optionally substituted by alkyl or cycloalkyl or aryl or heteroaryl or heterocyclyl, silyloxy optionally substituted by alkyl or aryl, silyl optionally substituted by alkoxy or alkyl or aryl, multiple degrees of substitution being allowed. Examples of “arylene” include, but are not limited to, benzene-1,4-diyl, naphthalene-1,8-diyl, and the like.

As used herein, “cycloalkyl” (used interchangeably with “aliphatic cyclic” herein) refers to a non-aromatic monovalent, monocyclic or polycyclic ring structure having a total of from 3 to 18 carbon ring atoms (but no heteroatoms) optionally possessing one or more degrees of unsaturation, optionally substituted with substituents such as, without limitation, nitro, cyano, halogen, perfluoroalkyl, aryl, heteroaryl, heterocyclyl, cycloalkyl, lower alkyl, lower alkoxy, lower alkylsulfanyl, lower alkylsulfenyl, lower alkylsulfonyl, oxo, hydroxy, mercapto, amino optionally substituted by alkyl or aryl or heteroaryl or heterocyclyl or cycloalkyl, aminocarbonyl (—NRC(O)R) optionally substituted by alkyl or aryl or heteroaryl or heterocyclyl or cycloalkyl, carboxy, acyl, acyloxy, alkoxycarbonyl, aryloxy, heteroaryloxy, heterocyclyloxy, aroyloxy, heteroaroyloxy, heterocycloyloxy, carbamoyl optionally substituted by alkyl or cycloalkyl or aryl or heteroaryl or heterocyclyl, aminosulfonyl optionally substituted by alkyl or cycloalkyl or aryl or heteroaryl or heterocyclyl, silyloxy optionally substituted by alkyl or aryl, silyl optionally substituted by alkoxy or alkyl or aryl, multiple degrees of substitution being allowed. “Cycloalkyl” includes by way of example cyclopropyl, cyclobutyl, cyclopentyl, cyclopentenyl, cyclohexyl, cyclohehexenyl, adamantanyl, norbornyl, nobornenyl, cycloheptyl, or cyclooctyl, and the like.

As used herein, the term “cycloalkylene” refers to an non-aromatic alicyclic divalent hydrocarbon radical having from three to twelve carbon atoms and optionally possessing one or more degrees of unsaturation, optionally substituted with substituents such as, but not limited to, nitro, cyano, halogen, perfluoroalkyl, aryl, heteroaryl, heterocyclyl, cycloalkyl, lower alkyl, lower alkoxy, lower alkylsulfanyl, lower alkylsulfenyl, lower alkylsulfonyl, oxo, hydroxy, mercapto, amino optionally substituted by alkyl or aryl or heteroaryl or heterocyclyl or cycloalkyl, aminocarbonyl (—NRC(O)R) optionally substituted by alkyl or aryl or heteroaryl or heterocyclyl or cycloalkyl, carboxy, acyl, acyloxy, alkoxycarbonyl, aryloxy, heteroaryloxy, heterocyclyloxy, aroyloxy, heteroaroyloxy, heterocycloyloxy, carbamoyl optionally substituted by alkyl or cycloalkyl or aryl or heteroaryl or heterocyclyl, aminosulfonyl optionally substituted by alkyl or cycloalkyl or aryl or heteroaryl or heterocyclyl, silyloxy optionally substituted by alkyl or aryl, silyl optionally substituted by alkoxy or alkyl or aryl, multiple degrees of substitution being allowed. Examples of “cycloalkylene” as used herein include, but are not limited to, cyclopropyl-1,1-diyl, cyclopropyl-1,2-diyl, cyclobutyl-1,2-diyl, cyclopentyl-1,3-diyl, cyclohexyl-1,4-diyl, cycloheptyl-1,4-diyl, or cyclooctyl-1,5-diyl, and the like.

The terms “heterocycle” and “heterocyclic” as used herein are used interchangeably to refer to a three to twelve-membered heterocyclic ring optionally possessing one or more degrees of unsaturation, containing one or more heteroatomic substitutions selected from —S—, —SO—, —SO₂—, —O—, or —N—, optionally substituted with substitutents, including, but not limited to, nitro, cyano, halogen, perfluoroalkyl, aryl, heteroaryl, heterocyclyl, cycloalkyl, lower alkyl, lower alkoxy, lower alkylsulfanyl, lower alkylsulfenyl, lower alkylsulfonyl, oxo, hydroxy, mercapto, amino optionally substituted by alkyl or aryl or heteroaryl or heterocyclyl or cycloalkyl, aminocarbonyl (—NRC(O)R) optionally substituted by alkyl or aryl or heteroaryl or heterocyclyl or cycloalkyl, carboxy, acyl, acyloxy, alkoxycarbonyl, aryloxy, heteroaryloxy, heterocyclyloxy, aroyloxy, heteroaroyloxy, heterocycloyloxy, carbamoyl optionally substituted by alkyl or cycloalkyl or aryl or heteroaryl or heterocyclyl, aminosulfonyl optionally substituted by alkyl or cycloalkyl or aryl or heteroaryl or heterocyclyl, silyloxy optionally substituted by alkyl or aryl, silyl optionally substituted by alkoxy or alkyl or aryl, multiple degrees of substitution being allowed. Such a ring optionally may be fused to one or more of another “heterocyclic” ring(s). Examples of “heterocyclic” include, but are not limited to, pyrrole, furan, thiophene, imidazole, oxazole, thiazole, pyrazole, 3-pyrroline, pyrrolidine, pyridine, pyrimidine, purine, quinoline, isoquinoline, carbazole, tetrahydrofuran, 1,4-dioxane, 1,3-dioxane, piperidine, pyrrolidine, morpholine, piperazine and the like.

As used herein, the term “C-linked heterocycle” means a heterocycle that is bonded through a carbon atom, e.g. —(CH₂)ₙ-heterocycle where n is 1, 2 or 3 or —C<heterocycle where C<represents a carbon atom in a heterocycle ring. Similarly, R moieties that are N-linked heterocycles mean a heterocycle that is bonded through a heterocycle ring nitrogen atom, e.g. —N<heterocycle where N<represents a nitrogen atom in a heterocycle ring.

Examples of heterocycles include, but are not limited to, pyridyl, thiazolyl, tetrahydrothiophenyl, sulfur oxidized tetrahydrothiophenyl, pyrimidinyl, furanyl, thienyl, pyrrolyl, pyrazolyl, imidazolyl, tetrazolyl, benzofuranyl, thianaphthalenyl, indolyl, indolenyl, quinolinyl, isoquinolinyl, benzimidazolyl, piperidinyl, 4-piperidonyl, pyrrolidinyl, 2-pyrrolidonyl, pyrrolinyl, tetrahydrofuranyl, tetrahydroquinolinyl, tetrahydroisoquinolinyl, decahydroquinolinyl, octahydroisoquinolinyl, azocinyl, triazinyl, 6H-1,2,5-thiadiazinyl, 2H,6H-1,5,2-dithiazinyl, thienyl, thianthrenyl, pyranyl, isobenzofuranyl, chromenyl, xanthenyl, phenoxathiinyl, 2H-pyrrolyl, isothiazolyl, isoxazolyl, pyrazinyl, pyridazinyl, indolizinyl, isoindolyl, 3H-indolyl, 1H-indazoly, purinyl, 4H-quinolizinyl, phthalazinyl, naphthyridinyl, quinoxalinyl, quinazolinyl, cinnolinyl, pteridinyl, 4aH-carbazolyl, carbazolyl, beta-carbolinyl, phenanthridinyl, acridinyl, pyrimidinyl, phenanthrolinyl, phenazinyl, phenothiazinyl, furazanyl, phenoxazinyl, isochromanyl, chromanyl, imidazolidinyl, imidazolinyl, pyrazolidinyl, pyrazolinyl, piperazinyl, indolinyl, isoindolinyl, quinuclidinyl, morpholinyl, oxazolidinyl, benzotriazolyl, benzisoxazolyl, oxindolyl, benzoxazolinyl, and isatinoyl.

By way of example and not limitation, carbon bonded heterocycles are bonded at position 2, 3, 4, 5, or 6 of a pyridine, position 3, 4, 5, or 6 of a pyridazine, position 2, 4, 5, or 6 of a pyrimidine, position 2, 3, 5, or 6 of a pyrazine, position 2, 3, 4, or 5 of a furan, tetrahydrofuran, thiofuran, thiophene, pyrrole or tetrahydropyrrole, position 2, 4, or 5 of an oxazole, imidazole or thiazole, position 3, 4, or 5 of an isoxazole, pyrazole, or isothiazole, position 2 or 3 of an aziridine, position 2, 3, or 4 of an azetidine, position 2, 3, 4, 5, 6, 7, or 8 of a quinoline or position 1, 3, 4, 5, 6, 7, or 8 of an isoquinoline. Carbon bonded heterocycles include 2-pyridyl, 3-pyridyl, 4-pyridyl, 5-pyridyl, 6-pyridyl, 3-pyridazinyl, 4-pyridazinyl, 5-pyridazinyl, 6-pyridazinyl, 2-pyrimidinyl, 4-pyrimidinyl, 5-pyrimidinyl, 6-pyrimidinyl, 2-pyrazinyl, 3-pyrazinyl, 5-pyrazinyl, 6-pyrazinyl, 2-thiazolyl, 4-thiazolyl, or 5-thiazolyl.

By way of example and not limitation, nitrogen bonded heterocycles are bonded at the nitrogen atom or position 1 of an aziridine, azetidine, pyrrole, pyrrolidine, 2-pyrroline, 3-pyrroline, imidazole, imidazolidine, 2-imidazoline, 3-imidazoline, pyrazole, pyrazoline, 2-pyrazoline, 3-pyrazoline, piperidine, piperazine, indole, indoline, 1H-indazole, position 2 of a isoindole, or isoindoline, position 4 of a morpholine, and position 9 of a carbazole, or beta-carboline. Typically, nitrogen bonded heterocycles include 1-aziridyl, 1-azetedyl, 1-pyrrolyl, 1-imidazolyl, 1-pyrazolyl, and 1-piperidinyl and tautomers of any of these.

As used herein, the term “heterocyclylene” refers to a three to twelve-membered heterocyclic ring diradical optionally having one or more degrees of unsaturation containing one or more heteroatoms selected from S, SO, SO₂, O, or N, optionally substituted with substituents such as, without limitation, lower nitro, cyano, halogen, perfluoroalkyl, aryl, heteroaryl, heterocyclyl, cycloalkyl, lower alkyl, lower alkoxy, lower alkylsulfanyl, lower alkylsulfenyl, lower alkylsulfonyl, oxo, hydroxy, mercapto, amino optionally substituted by alkyl or aryl or heteroaryl or heterocyclyl or cycloalkyl, aminocarbonyl (—NRC(O)R) optionally substituted by alkyl or aryl or heteroaryl or heterocyclyl or cycloalkyl, carboxy, acyl, acyloxy, alkoxycarbonyl, aryloxy, heteroaryloxy, heterocyclyloxy, aroyloxy, heteroaroyloxy, heterocycloyloxy, carbamoyl optionally substituted by alkyl or cycloalkyl or aryl or heteroaryl or heterocyclyl, aminosulfonyl optionally substituted by alkyl or cycloalkyl or aryl or heteroaryl or heterocyclyl, silyloxy optionally substituted by alkyl or aryl, silyl optionally substituted by alkoxy or alkyl or aryl, multiple degrees of substitution being allowed. Such a ring may be optionally fused to one or more benzene rings or to one or more of another “heterocyclic” rings or cycloalkyl rings. Examples of “heterocyclylene” include, but are not limited to, tetrahydrofuran-2,5-diyl, morpholine-2,3-diyl, pyran-2,4-diyl, 1,4-dioxane-2,3-diyl, 1,3-dioxane-2,4-diyl, piperidine-2,4-diyl, piperidine-1,4-diyl, pyrrolidine-1,3-diyl, morpholine-2,4-diyl, piperazine-1,4-dyil, and the like.

As used herein, the term “heteroaryl” refers to a five-to seven-membered aromatic ring, or to a polycyclic heterocyclic aromatic ring, containing one or more nitrogen, oxygen, or sulfur heteroatoms, where N-oxides and sulfur monoxides and sulfur dioxides are permissible heteroaromatic substitutions, optionally substituted with substituents including, but not limited to, nitro, cyano, halogen, perfluoroalkyl, aryl, heteroaryl, heterocyclyl, cycloalkyl, lower alkyl, lower alkoxy, lower alkylsulfanyl, lower alkylsulfenyl, lower alkylsulfonyl, oxo, hydroxy, mercapto, amino optionally substituted by alkyl or aryl or heteroaryl or heterocyclyl or cycloalkyl, aminocarbonyl (—NRC(O)R) optionally substituted by alkyl or aryl or heteroaryl or heterocyclyl or cycloalkyl, carboxy, acyl, acyloxy, alkoxycarbonyl, aryloxy, heteroaryloxy, heterocyclyloxy, aroyloxy, heteroaroyloxy, heterocycloyloxy, carbamoyl optionally substituted by alkyl or cycloalkyl or aryl or heteroaryl or heterocyclyl, aminosulfonyl optionally substituted by alkyl or cycloalkyl or aryl or heteroaryl or heterocyclyl, silyloxy optionally substituted by alkyl or aryl, silyl optionally substituted by alkoxy or alkyl or aryl, multiple degrees of substitution being allowed. For polycyclic aromatic ring systems, one or more of the rings may contain one or more heteroatoms. Examples of “heteroaryl” used herein are furan, thiophene, pyrrole, imidazole, pyrazole, triazole, tetrazole, thiazole, oxazole, isoxazole, oxadiazole, thiadiazole, isothiazole, pyridine, pyridazine, pyrazine, pyrimidine, quinoline, isoquinoline, quinazoline, benzofuran, benzothiophene, indole, and indazole, and the like.

As used herein, the term “heteroarylene” refers to a five-to seven-membered aromatic ring diradical, or to a polycyclic heterocyclic aromatic ring diradical, containing one or more nitrogen, oxygen, or sulfur heteroatoms, where N-oxides and sulfur monoxides and sulfur dioxides are permissible heteroaromatic substitutions, optionally substituted with substituents including, but not limited to, nitro, cyano, halogen, perfluoroalkyl, aryl, heteroaryl, heterocyclyl, cycloalkyl, lower alkyl, lower alkoxy, lower alkylsulfanyl, lower alkylsulfenyl, lower alkylsulfonyl, oxo, hydroxy, mercapto, amino optionally substituted by alkyl or aryl or heteroaryl or heterocyclyl or cycloalkyl, aminocarbonyl (—NRC(O)R) optionally substituted by alkyl or aryl or heteroaryl or heterocyclyl or cycloalkyl, carboxy, acyl, acyloxy, alkoxycarbonyl, aryloxy, heteroaryloxy, heterocyclyloxy, aroyloxy, heteroaroyloxy, heterocycloyloxy, carbamoyl optionally substituted by alkyl or cycloalkyl or aryl or heteroaryl or heterocyclyl, aminosulfonyl optionally substituted by alkyl or cycloalkyl or aryl or heteroaryl or heterocyclyl, silyloxy optionally substituted by alkyl or aryl, silyl optionally substituted by alkoxy or alkyl or aryl, multiple degrees of substitution being allowed. For polycyclic aromatic ring system diradicals, one or more of the rings may contain one or more heteroatoms. Examples of “heteroarylene” used herein are furan-2,5-diyl, thiophene-2,4-diyl, 1,3,4-oxadiazole-2,5-diyl, 1,3,4-thiadiazole-2,5-diyl, 1,3-thiazole-2,4-diyl, 1,3-thiazole-2,5-diyl, pyridine-2,4-diyl, pyridine-2,3-diyl, pyridine-2,5-diyl, pyrimidine-2,4-diyl, quinoline-2,3-diyl, and the like.

As used herein, the term “direct bond”, where part of a structural variable specification, refers to the direct joining of the substituents flanking (preceding and succeeding) the variable taken as a “direct bond”.

As used herein, the term “O-linked moiety” means a moiety that is bonded through an oxygen atom. Thus, when an R group is an O-linked moiety, that R is bonded through oxygen and it thus can be an ether, an ester (e.g., —O—C(O)-optionally substituted alkyl), a carbonate or a carbamate (e.g., —O—C(O)—NH₂ or —O—C(O)—NH-optionally substituted alkyl). Similarly, the term “S-linked moiety” means a moiety that is bonded through a sulfur atom. Thus, when an R group is an S-linked moiety, that R is bonded through sulfur and it thus can be a thioether (e.g., —S-optionally substituted alkyl), a thioester (—S—C(O)-optionally substituted alkyl) or a disulfide (e.g., —S—S-optionally substituted alkyl). The term “N-linked moiety” means a moiety that is bonded through a nitrogen atom. Thus, when an R group is an N-linked moiety, the R group is bonded through nitrogen and one or more of these can thus be an N-linked amino acid such as —NH—CH2-COOH, a carbamate such as —NH—C(O)—O-optionally substituted alkyl, an amine such as —NH-optionally substituted alkyl, an amide such as —NH—C(O)-optionally substituted alkyl or —N₃. The term “C-linked moiety” means a moiety that is bonded through a carbon atom. When one or more R group is bonded through carbon, one or more of these thus can be -optionally substituted alkyl such as —CH₂—CH₂—O—CH₃, —C(O)-optionally substituted alkyl hydroxyalkyl, mercaptoalkyl, aminoalkyl or ═CH-optionally substituted alkyl.

The term “alkoxy” as used herein refers to the group RₐO—, where Rₐ is alkyl.

The term “alkenyloxy” as used herein refers to the group RₐO—, where Rₐ is alkenyl.

The term “alkynyloxy” as used herein refers to the group RₐO—, where Rₐ is alkynyl.

The term “alkylsulfanyl” as used herein refers to the group RₐS—, where Rₐ is alkyl.

The term “alkenylsulfanyl” as used herein refers to the group RₐS—, where Rₐ is alkenyl.

The term “alkynylsulfanyl” as used herein refers to the group RₐS—, where Rₐ is alkynyl.

The term “alkylsulfenyl” as used herein refers to the group RₐS(O)—, where Rₐ is alkyl.

The term “alkenylsulfenyl” as used herein refers to the group RₐS(O)—, where Rₐ is alkenyl.

The term “alkynylsulfenyl” as used herein refers to the group RₐS(O)—, where Rₐ is alkynyl.

The term “alkylsulfonyl” as used herein refers to the group RₐSO₂—, where Rₐ is alkyl.

The term “alkenylsulfonyl” as used herein refers to the group RₐSO₂—, where Rₐ is alkenyl.

The term “alkynylsulfonyl” as used herein refers to the group RₐSO₂—, where Rₐ is alkynyl.

The term “acyl” as used herein refers to the group RₐC(O)—, where Rₐ is alkyl, alkenyl, alkynyl, cycloalkyl, cycloalkenyl, aryl, heteroaryl, or heterocyclyl.

The term “aroyl” as used herein refers to the group RₐC(O)—, where Rₐ is aryl.

The term “heteroaroyl” as used herein refers to the group RₐC(O)—, where Rₐ is heteroaryl.

The term “heterocycloyl” as used herein refers to the group RₐC(O)—, where Rₐ is heterocyclyl.

The term “alkoxycarbonyl” as used herein refers to the group RₐOC(O)—, where Rₐ is alkyl.

The term “acyloxy” as used herein refers to the group RₐC(O)O—, where Rₐ is alkyl, alkenyl, alkynyl, cycloalkyl, cycloalkenyl, aryl, heteroaryl, or heterocyclyl.

The term “aroyloxy” as used herein refers to the group RₐC(O)O—, where Rₐ is aryl.

The term “heteroaroyloxy” as used herein refers to the group RₐC(O)O—, where Rₐ is heteroaryl.

The term “heterocycloyloxy” as used herein refers to the group RₐC(O)O—, where Rₐ is heterocyclyl.

The term “substituted” as used herein refers to substitution with the named substituent or substituents, multiple degrees of substitution being allowed unless otherwise stated.

The terms “contain” or “containing” can as used herein refers to in-line substitutions at any position along the above defined alkyl, alkenyl, alkynyl or cycloalkyl substituents with one or more of any of O, S, SO, SO₂, N, or N-alkyl, including, for example, —CH₂—O—CH₂, —CH₂—SO₂—CH₂, —CH₂—NH—CH₃ and so forth.

The term “oxo” as used herein refers to the substituent ═O.

The term “halogen” or “halo” as used herein includes iodine, bromine, chlorine and fluorine.

The term “mercapto” as used herein refers to the substituent —SH.

The term “carboxy” as used herein refers to the substituent —COOH.

The term “cyano” as used herein refers to the substituent —CN.

The term “aminosulfonyl” as used herein refers to the substituent —SO₂NH₂.

The term “carbamoyl” as used herein refers to the substituent —C(O)NH₂.

The term “sulfanyl” as used herein refers to the substituent —S—.

The term “sulfenyl” as used herein refers to the substituent —S(O)—.

The term “sulfonyl” as used herein refers to the substituent —S(O)₂—.

The term “ethoxy” as used herein refers to the substituent —O—CH₂CH₃.

The term “methoxy” as used herein refers to the substituent —O—CH₃.

As used herein, the term “optionally” means that the subsequently described event(s) may or may not occur, and includes both event(s) which occur and events that do not occur.

As used herein, the term “configuration” refers to the three-dimensional shape of a molecule. In order to represent three-dimensional configurations on a two-dimensional surface, perspective drawings in which the direction of a bond is specified by the line connecting the bonded atoms are used. Formula III shows an illustrative perspective drawing:

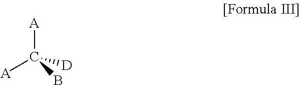

In formula III, the focus of configuration is a carbon (C) atom so the lines specifying bond directions will originate there. A simple straight line represents a bond lying approximately in the surface plane, as shown by the two bonds to substituent “A.” A wedge shaped bond is directed in front of this plane (thick end toward the viewer), as shown by the bond to substituent “B.” A hatched bond is directed in back of the plane (away from the viewer), as shown by the bond to substituent “D.” A dashed bond represents a single or double bond which can be in the cis or trans configuration.

### Stereochemistry

According to some embodiments, the stereochemistry of the chiral centers (marked by “*”) represents all possible combinations in terms of relative and absolute chemistry. Accordingly, it may represent either racemic enantiomers or pure enantiomers.

The term “racemate” as used herein refers to an equimolar mixture of two optically active components that neutralize the optical effect of each other and is therefore optically inactive.

As used herein, an “enantiomer” refers to one of a pair of optical isomers containing one or more symmetric carbons (C*) whose molecular configurations have left- and right-hand (chiral) forms. Enantiomers have identical physical properties, except for the direction of rotation of the plane of polarized light. For example, the two 2-methyl-1-butanols have identical melting points, boiling points, densities, refractive indexes, and any other physical constant one might measure, expect that one rotates the plane-polarized light to the right, the other to the left. Only the direction of rotation is different; the amount of rotation is the same. Enantiomers have identical chemical properties except toward optically active reagents. The atoms of each enantiomer that undergo attack in each enantiomer are influenced in their reactivity by exactly the same combination of substituents. The reagent approaching either kind of molecule encounters the same environment, except that one environment is the mirror image of the other. In the case of a reagent that is itself optically active, the influences exerted on the reagent are not identical in the attack on the two enantiomers, and reaction rates will be different; in some cases the reaction with one isomer does not take place at all. In biological systems, such stereochemical specificity is the rule rather than the exception, since enzymes, and most of the compounds they work on, are optically active. Enantiomers show different properties (physical or chemical) only in a chiral medium. Polarized light provides such a medium, and in it enantiomers differ in a physical property: direction of the rotation of the light. They also may differ in solubility in an optically active solvent, or in adsorption on an optically active surface. For enantiomers to react at different rates, the necessary chiral medium can be provided in a number of ways: by an optically active reagent; by a chiral solvent, or the chiral surface of a catalyst. The terms “optically active reagent” or “chiral reagent” refer to reaction under any chiral condition. The terms “optically inactive reagent” or “achiral reagent” refer to reaction in the absence of a chiral medium.

When named by the spatial configuration of its atoms, optical isomers conventionally are designated dextro (D) and levo (L) because they compare to each other structurally as do the right and left hand when the carbon atoms are lined up, i.e., they are mirror images of each other. Several pairs of enantiomers are possible depending on the number of asymmetric carbon atoms in the molecule. Compounds in which an asymmetric carbon is present display optical rotation, meaning the change of direction of the plane of polarized light to either the right or the left as it passes through a molecule containing one or more asymmetric carbon atoms.

Each chiral center is labeled R or S according to a system by which its substituents are each designated a priority according to the Cahn Ingold Prelog priority rules (CIP), based on atomic number. If the center is oriented so that the lowest priority of the four is pointed away from a viewer, the viewer will see two possibilities: if the priority of the remaining three substituents decreases in clockwise direction, it is labeled R (for Rectus), if it decreases in counterclockwise direction, it is S (for Sinister).

This system labels each chiral center in a molecule (and also has an extension to chiral molecules not involving chiral centers). Thus, it has greater generality than the D/L system, and can label, for example, an (R,R) isomer versus an (R,S)-diastereomer.

The R/S system has no fixed relation to the (+)/(−) system. An R isomer can be either dextrorotatory or levorotatory, depending on its exact substituents.

The R/S system also has no fixed relation to the D/L system. For example, the side-chain one of serine contains a hydroxyl group, —OH. If a thiol group, —SH, were swapped in for it, the D/L labeling would, by its definition, not be affected by the substitution. But this substitution would invert the molecule's R/S labeling, because the CIP priority of CH₂OH is lower than that for CO₂H but the CIP priority of CH₂SH is higher than that for CO₂H.

For this reason, the D/L system remains in common use in certain areas of biochemistry, such as amino acid and carbohydrate chemistry, because it is convenient to have the same chiral label for all of the commonly occurring structures of a given type of structure in higher organisms. In the D/L system, they are nearly all consistent-naturally occurring amino acids are nearly all L, while naturally occurring carbohydrates are nearly all D. In the R/S system, they are mostly S, but there are some common exceptions.

An enantiomer can be named by the direction in which it rotates the plane of polarized light. If it rotates the light clockwise (as seen by a viewer towards whom the light is traveling), that enantiomer is labeled (+). Its mirror-image is labeled (−). The (+) and (−) isomers have also been termed d- and l-, respectively (for dextrorotatory and levorotatory).

An optical isomer can be named by the spatial configuration of its atoms. The D/L system does this by relating the molecule to glyceraldehyde. Glyceraldehyde is chiral itself, and its two isomers are labeled D and L (typically typeset in small caps in published work). Certain chemical manipulations can be performed on glyceraldehyde without affecting its configuration, and its historical use for this purpose (possibly combined with its convenience as one of the smallest commonly used chiral molecules) has resulted in its use for nomenclature. In this system, compounds are named by analogy to glyceraldehyde, which, in general, produces unambiguous designations, but is easiest to see in the small biomolecules similar to glyceraldehyde. One example is the amino acid alanine, which has two optical isomers, and they are labeled according to which isomer of glyceraldehyde they come from. On the other hand, glycine, the amino acid derived from glyceraldehyde, has no optical activity, as it is not chiral (achiral). Alanine, however, is chiral.

The D/L labeling is unrelated to (+)/(−); it does not indicate which enantiomer is dextrorotatory and which is levorotatory. Rather, it says that the compound's stereochemistry is related to that of the dextrorotatory or levorotatory enantiomer of glyceraldehyde—the dextrorotatory isomer of glyceraldehyde is, in fact, the D isomer. Nine of the nineteen L-amino acids commonly found in proteins are dextrorotatory (at a wavelength of 589 nm), and D-fructose is also referred to as levulose because it is levorotatory.

A rule of thumb for determining the D/L isomeric form of an amino acid is the “CORN” rule. The groups: COOH, R, NH₂ and H (where R is a variant carbon chain) are arranged around the chiral center carbon atom. Sighting with the hydrogen atom away from the viewer, if these groups are arranged clockwise around the carbon atom, then it is the D-form. If counter-clockwise, it is the L-form.

As used herein, the term “absolute configuration” refers to the spatial arrangement of the atoms of a chiral molecular entity (or group) and its stereochemical description, for example, R or S.

The term “asymmetric” as used herein refers to lacking all symmetry elements (other than the trivial one of a one-fold axis of symmetry), i.e., belonging to the symmetry of point group C₁. The term has been used loosely (and incorrectly) to describe the absence of a rotation-reflection axis (alternating axis) in a molecule, i.e., as meaning chiral, and this usage persists in the traditional terms such as, but not limited to, asymmetric carbon atom, asymmetric synthesis, and asymmetric induction.

As used herein, the term “relative configuration” refers to the configuration of any stereogenic (asymmetric) center with respect to any other stereogenic center contained within the same molecular entity. Unlike absolute configuration, relative configuration is reflection-invariant. Relative configuration, distinguishing diastereoisomers may be denoted by the configurational descriptors R*,R* (or l) and R*,S* (or u) meaning, respectively, that the two centers have identical or opposite configurations. For molecules with more than two asymmetric centers, the prefix rel- may be used in front of the name of one enantiomer where R and S have been used. If any centers have known absolute configuration then only R* and S* can be used for the relative configuration. For example, two different molecules Xabcd and Xabce may be said to have the same relative configurations if e takes the position of d in the tetrahedral arrangement of ligands around X (i.e., the pyramidal fragments Xabc are superposable). Similarly, the enantiomer of Xabce may be said to have the opposite relative configuration to Xabcd. The terms may be applied to chiral molecular entities with central atoms other than carbon but are limited to cases where the two related molecules differ in a single ligand. These definitions can be generalized to include stereogenic units other than asymmetric centers.

As used herein, the term “stereogenic unit” (or “stereogen” or “stereoelement”) refers to a grouping within a molecular entity that may be considered a focus of stereoisomerism. At least one of these must be present in every enantiomer (though the presence of stereogenic units does not conversely require the corresponding chemical species to be chiral). Three basic types are recognized for molecular entities involving atoms having not more than four substituents: (a) a grouping of atoms consisting of a central atom and distinguishable ligands, such that the interchange of any two of the substituents leads to a stereoisomer. An asymmetric atom (chirality center) is the traditional example of this stereogenic unit; (b) a chain of four non-coplanar atoms (or rigid groups) in a stable conformation, such that an imaginary or real (restricted) rotation (with a change of sign of the torsion angle) about the central bond leads to a steroisomer; and (c) a grouping of atoms consisting of a double bond with substituents which give rise to cis-trans isomerism.

As used herein, the term “chiral” is used to describe an object that is nonsuperposable on its mirror image and therefore has the property of chirality.

As used herein, the term “chirality” refers to the geometric property of a rigid object (or spatial arrangement of points or atoms) of being non-superposable on its mirror image; such an object has no symmetry elements of the second kind (a mirror plane, σ=S₁, a center of inversion, i=S₂, a rotation-reflection axis, S₂ₙ). If the object is superposable on its mirror image the object is described as being achiral.

As used herein, the term “chirality axis” refers to an axis about which a set of ligands is held so that it results in a spatial arrangement which is not superposable on its mirror image. For example, with an allene abC═C═Ccd the chiral axis is defined by the C═C═C bonds; and with an ortho-substituted biphenyl C-1, C-1′, C-4 and C-4′ lie on the chiral axis.

As used herein, the term “chirality center” refers to an atom holding a set of ligands in a spatial arrangement, which is not superposable on its mirror image. A chirality center may be considered a generalized extension of the concept of the asymmetric carbon atom to central atoms of any element.

As used herein, the terms “chiroptic” or “chiroptical” refer to the optical techniques (using refraction, absorption or emission of anisotropic radiation) for investigating chiral substances (for example, measurements of optical rotation at a fixed wavelength, optical rotary dispersion (ORD), circular dichroism (CD) and circular polarization of luminescence (CPL).

As used herein, the term “chirotopic” refers to an atom (or point, group, face, etc. in a molecular model) that resides within a chiral environment. One that resides within an achiral environment has been called achirotopic.

As used herein, the terms “cis” and “trans” are descriptors which show the relationship between two ligands attached to separate atoms that are connected by a double bond or are contained in a ring. The two ligands are said to be located cis to each other if they lie on the same side of a plane. If they are on opposite sides, their relative position is described as trans. The appropriate reference plane of a double bond is perpendicular to that of the relevant σ-bonds and passes through the double bond. For a ring (the ring being in a conformation, real or assumed, without re-entrant angles at the two substituted atoms) it is the mean place of the ring(s). For alkenes the terms cis and trans may be ambiguous and have therefore generally have been replaced by the E, Z convention for the nomenclature of organic compounds. If there are more than two entities attached to the ring the use of cis and trans requires the definition of a reference substituent (see IUPAC, Nomenclature of Organic Chemistry, Sections A, B, C, D, E, F and H, Pergamon Press, 1979, p. 478, Rule E-2.3.3, E-2.3.4; IUPAC, A Guide to IUPAC Nomenclature of Organic Chemistry, Blackwell Scientific Publications, 1993, pp. 149-151, Rule R-7.1.1).

As used herein, the terms “cis-trans isomers” refer to stereoisomeric olefins or cycloalkanes (or hetero-analogues) which differ in the positions of atoms (or groups) relative to a reference plane: in the cis-isomer the atoms are on the same side, in the trans-isomer they are on opposite sides. For example:

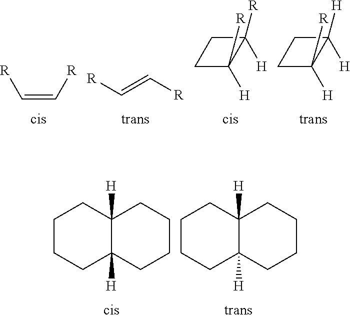

As used herein, the term “diastereoisomerism” refers to stereoisomerism other than enantiomerism. Diastereoisomers (or diastereomers) are stereoisomers not related as mirror images. Diastereoisomers are characterized by differences in physical properties, and by some differences in chemical behavior towards achiral as well as chiral reagents. Diastereomers have similar chemical properties, since they are members of the same family. Their chemical properties are not identical, however. Diastereomers have different physical properties: different melting points, boiling points, solubilities in a given solvent, densities, refractive indexes, and so on. Diastereomers also differ in specific rotation; they may have the same or opposite signs of rotation, or some may be inactive. The presence of two chiral centers can lead to the existence of as many as four stereoisomers. For compounds containing three chiral centers, there could be as many as eight stereoisomers; for compounds containing four chiral centers, there could be as many as sixteen stereoisomers, and so on. The maximum number of stereoisomers that can exist is equal to 2ⁿ, where n is the number of chiral centers. The term “diastereotopic” refers to constitutionally equivalent atoms or groups of a molecule which are not symmetry related. Replacement of one of two diastereotopic atoms or groups results in the formation of one of a pair of diastereoisomers. For example, the two hydrogen atoms of the methylene group C-3 are diastereotopic.

The term “isomer” as used herein refers to one of two or more molecules having the same number and kind of atoms and hence the same molecular weight, but differing in respect to the arrangement or configuration of the atoms. Stereoisomers are isomers that are different from each other only in the way the atoms are oriented in space (but are like one another with respect to which atoms are joined to which other atoms).

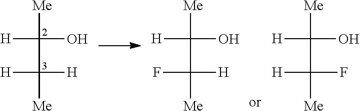 [SMILES: CCC(C)O ; CC(O)C(C)F ; CC(O)C(C)F]

According to this definition, cis-trans isomerism is a form of diastereoisomerism.

As used herein, the term “superposability” refers to the ability to bring two particular stereochemical formulae (or models) into coincidence (or to be exactly superposable in space, and for the corresponding molecular entities or objects to become exact replicas of each other) by no more than translation and rigid rotation.

### Compositions

According to another aspect, the described invention provides pharmaceutical compositions comprising a synthetic biologically active mangrolide A compound or an analog or derivative thereof and a pharmaceutically acceptable carrier.

The term “active” as used herein refers to having pharmacological or biological activity or affect. The term “active ingredient” (“AT”, “active pharmaceutical ingredient”, or “bulk active”) is the substance in a drug that is pharmaceutically active.

The terms “formulation” and “composition” are used interchangeably herein to refer to a product of the described invention that comprises all active and inert ingredients. The terms “pharmaceutical formulation” or “pharmaceutical composition” as used herein refer to a formulation or composition that is employed to prevent, reduce in intensity, cure or otherwise treat a target condition or disease.

As used herein, the term “binder” refers to substances that bind or “glue” powders together and make them cohesive by forming granules, thus serving as the “adhesive” in the formulation. Binders add cohesive strength already available in the diluent or bulking agent. Exemplary binders include sugars such as sucrose; starches derived from wheat, corn rice and potato; natural gums such as acacia, gelatin and tragacanth; derivatives of seaweed such as alginic acid, sodium alginate and ammonium calcium alginate; cellulosic materials such as methylcellulose and sodium carboxymethylcellulose and hydroxypropylmethylcellulose; polyvinylpyrrolidone; and inorganics such as magnesium aluminum silicate. The amount of binder in the composition can range from about 2 to about 20% by weight of the composition, more preferably from about 3 to about 10% by weight, even more preferably from about 3 to about 6% by weight.

As used herein, the term “capsule” refers to a special container or enclosure made of methyl cellulose, polyvinyl alcohols, or denatured gelatins or starch for holding or containing compositions comprising the active ingredients. Hard shell capsules are typically made of blends of relatively high gel strength bone and pork skin gelatins. The capsule itself may contain small amounts of dyes, opaquing agents, plasticizers and preservatives.

As used herein, the term “coloring agents” refers to excipients that provide coloration to the composition or the dosage form. Such excipients can include food grade dyes and food grade dyes adsorbed onto an Exemplary adsorbent such as clay or aluminum oxide. The amount of the coloring agent can vary from about 0.1 to about 5% by weight of the composition, preferably from about 0.1 to about 1%.

As used herein, the term “diluent” refers to substances that usually make up the major portion of the composition or dosage form. Exemplary diluents include sugars such as lactose, sucrose, mannitol and sorbitol; starches derived from wheat, corn, rice and potato; and celluloses such as microcrystalline cellulose. The amount of diluent in the composition can range from about 10 to about 90% by weight of the total composition, preferably from about 25 to about 75%, more preferably from about 30 to about 60% by weight, even more preferably from about 12 to about 60%.

As used herein, the term “disintegrant” refers to materials added to the composition to help it break apart (disintegrate) and release the medicaments. Exemplary disintegrants include starches; “cold water soluble” modified starches such as sodium carboxymethyl starch; natural and synthetic gums such as locust bean, karaya, guar, tragacanth and agar; cellulose derivatives such as methylcellulose and sodium carboxymethylcellulose; microcrystalline celluloses and cross-linked microcrystalline celluloses such as sodium croscarmellose; alginates such as alginic acid and sodium alginate; clays such as bentonites; and effervescent mixtures. The amount of disintegrant in the composition can range from about 2 to about 15% by weight of the composition, more preferably from about 4 to about 10% by weight.

As used herein, the term “glident” refers to material that prevents caking and improves the flow characteristics of granulations, so that flow is smooth and uniform. Exemplary glidents include silicon dioxide and talc. The amount of glident in the composition can range from about 0.1% to about 5% by weight of the total composition, preferably from about 0.5 to about 2% by weight.

As used herein, the term “lubricant” refers to a substance added to the dosage form to enable the tablet, granules, etc. after it has been compressed, to release from the mold or die by reducing friction or wear. Exemplary lubricants include metallic stearates such as magnesium stearate, calcium stearate or potassium stearate; stearic acid; high melting point waxes; and water soluble lubricants such as sodium chloride, sodium benzoate, sodium acetate, sodium oleate, polyethylene glycols and d′l-leucine. Lubricants are usually added at the very last step before compression, since they must be present on the surfaces of the granules and in between them and the parts of the tablet press. The amount of lubricant in the composition can range from about 0.2 to about 5% by weight of the composition, preferably from about 0.5 to about 2%, more preferably from about 0.3 to about 1.5% by weight.

As used herein, the term “oral gel” refers to the active ingredients dispersed or solubilized in a hydrophillic semi-solid matrix.

As used herein, the term “tablet” refers to a compressed or molded solid dosage form containing the active ingredients with suitable diluents. The tablet can be prepared by compression of mixtures or granulations obtained by wet granulation, dry granulation or by compaction.

As used herein, the term “therapeutic amount” refers to the amount of natural or fully synthetic biologically active Mangrolide A necessary or sufficient to realize a desired biologic effect. Combined with the teachings provided herein, by choosing among the various active compounds and weighing factors such as potency, relative bioavailability, patient body weight, severity of adverse side-effects and preferred mode of administration, an effective prophylactic or therapeutic treatment regimen may be planned which does not cause substantial toxicity and yet is effective to treat the particular subject. The effective amount for any particular application may vary depending on such factors as the disease or condition being treated, the particular described compound, the size of the subject, or the severity of the disease or condition. One of ordinary skill in the art may determine empirically the therapeutically effective amount of a particular described compound and/or other therapeutic agent without necessitating undue experimentation. It is generally preferred that a maximum dose be used, that is, the highest safe dose according to some medical judgment. The terms “dose” and “dosage” are used interchangeably herein.

For any compound described herein the therapeutically effective amount can be initially determined from preliminary in vitro studies and/or animal models. A therapeutically effective dose can also be determined from human data for natural or fully synthetic, biologically active mangrolide A. The applied dose can be adjusted based on the relative bioavailability and potency of the administered compound. Adjusting the dose to achieve maximal efficacy based on the methods described above and other methods as are well-known in the art is within the capabilities of the ordinarily skilled artisan.

The formulations of antibacterial compounds may be administered in pharmaceutically acceptable solutions, which may routinely contain pharmaceutically acceptable concentrations of salt, buffering agents, preservatives, compatible carriers, adjuvants, and optionally other therapeutic agents.

According to another embodiment, the compositions of the described invention can further include one or more additional compatible active ingredients. “Compatible” as used herein means that the components of such a composition are capable of being combined with each other in a manner such that there is no interaction that would substantially reduce the efficacy of the composition under ordinary use conditions. As used herein, the phrase “additional active ingredient” refers to an agent, other than Mangrolide A of the described composition, that exerts a pharmacological, or any other beneficial activity.

### Pharmaceutically Acceptable Carrier

The term “pharmaceutically-acceptable carrier” as used herein refers to one or more compatible solid or liquid filler, diluents or encapsulating substances which are Exemplary for administration to a human or other vertebrate animal. The term “carrier” as used herein refers to an organic or inorganic ingredient, natural or synthetic, with which the active ingredient is combined to facilitate the application. According to some embodiments, the carrier can be inert, or it can possess pharmaceutical benefits.

The components of the pharmaceutical compositions also are capable of being commingled in a manner such that there is no interaction which would substantially impair the desired pharmaceutical efficiency.

The carrier can be liquid or solid and is selected with the planned manner of administration in mind to provide for the desired bulk, consistency, etc., when combined with an active and the other components of a given composition.

### Administration

For use in therapy, a therapeutic amount of a natural or fully synthetic, biologically active mangrolide A may be administered to a subject by any mode. Administering the pharmaceutical composition may be accomplished by any means known to the skilled artisan. Routes of administration include, but are not limited to, parenteral oral, buccal, topical, by inhalation or insufflation (i.e., through the mouth or through the nose), or rectal.

The term “parenteral” as used herein refers to introduction into the body by way of an injection (i.e., administration by injection), including, for example, subcutaneously (i.e., an injection beneath the skin), intramuscularly (i.e., an injection into a muscle); intravenously (i.e., an injection into a vein), intrathecally (i.e., an injection into the space around the spinal cord), intrasternal injection, or infusion techniques. A parenterally administered composition of the present invention is delivered using a needle. Injectable preparations, such as sterile injectable aqueous or oleaginous suspensions, may be formulated according to the known art using Exemplary dispersing or wetting agents and suspending agents.

The compositions of the present invention may be in the form of a sterile injectable aqueous solution or oleaginous suspension. A solution generally is considered as a homogeneous mixture of two or more substances; it is frequently, though not necessarily, a liquid. In a solution, the molecules of the solute (or dissolved substance) are uniformly distributed among those of the solvent. A suspension is a dispersion in which a finely-divided species is combined with another species, with the former being so finely divided and mixed that it doesn't rapidly settle out. The term “dispersion”, as used herein, refers to a two-phase system, in which one phase is distributed as particles or droplets in the second, or continuous phase. In these systems, the dispersed phase frequently is referred to as the discontinuous or internal phase, and the continuous phase is called the external phase or dispersion medium. For example, in coarse dispersions, the particle size is 0.5 mm. In colloidal dispersions, size of the dispersed particle is in the range of approximately 1 nm to 0.5 mm. Molecular dispersion is a dispersion, in which the dispersed phase consists of individual molecules; if the molecules are less than colloidal size, the result is a true solution.

The compositions of the described invention also may be in the form of an emulsion. An emulsion is a two-phase system prepared by combining two immiscible liquid carriers, one of which is disbursed uniformly throughout the other and consists of globules that have diameters equal to or greater than those of the largest colloidal particles. The globule size is critical and must be such that the system achieves maximum stability. Usually, separation of the two phases will not occur unless a third substance, an emulsifying agent, is incorporated. Thus, a basic emulsion contains at least three components, the two immiscible liquid carriers and the emulsifying agent, as well as the active ingredient. Most emulsions incorporate an aqueous phase into a non-aqueous phase (or vice versa). However, it is possible to prepare emulsions that are basically non-aqueous, for example, anionic and cationic surfactants of the non-aqueous immiscible system glycerin and olive oil. Thus, the compositions of the invention may be in the form of an oil-in-water emulsion. The oily phase may be a vegetable oil, for example, olive oil or arachis oil, or a mineral oil, for example a liquid paraffin, or a mixture thereof. Exemplary emulsifying agents may be naturally-occurring gums, for example, gum acacia or gum tragacanth, naturally-occurring phosphatides, for example soy bean, lecithin, and esters or partial esters derived from fatty acids and hexitol anhydrides, for example sorbitan monooleate, and condensation products of the partial esters with ethylene oxide, for example, polyoxyethylene sorbitan monooleate.

According to some embodiments, the composition may be formulated for parenteral administration by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Pharmaceutical formulations for parenteral administration include aqueous solutions of the active compounds in water-soluble form. Additionally, suspensions of the active compounds may be prepared as appropriate oily injection suspensions. Exemplary lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension also may contain exemplary stabilizers or agents, which increase the solubility of the compounds to allow for the preparation of highly concentrated solutions. Alternatively, the active compounds may be in powder form for constitution with an exemplary vehicle, e.g., sterile pyrogen-free water, before use.

The pharmaceutical compositions also may comprise exemplary solid or gel phase carriers or excipients. Examples of such carriers or excipients include, but are not limited to, calcium carbonate, calcium phosphate, various sugars, starches, cellulose derivatives, gelatin, and polymers such as polyethylene glycols.

Exemplary liquid or solid pharmaceutical preparation forms are, for example, microencapsulated, and if appropriate, with one or more excipients, encochleated, coated onto microscopic gold particles, contained in liposomes, pellets for implantation into the tissue, or dried onto an object to be rubbed into the tissue. Such pharmaceutical compositions also may be in the form of granules, beads, powders, tablets, coated tablets, (micro)capsules, suppositories, syrups, emulsions, suspensions, creams, drops or preparations with protracted release of active compounds, in whose preparation excipients and additives and/or auxiliaries such as disintegrants, binders, coating agents, swelling agents, lubricants, or solubilizers are customarily used as described above. The pharmaceutical preparation forms can be used in a variety of drug delivery systems. For a brief review of methods for drug delivery, see Langer 1990 Science 249, 1527-1533, which is incorporated herein by reference.

Depending upon the structure, a compound of the described invention may be administered per se (neat) or, depending upon the structure of the antibiotic, in the form of a pharmaceutically acceptable salt. The antibiotics of the described invention may form pharmaceutically acceptable salts with organic or inorganic acids, or organic or inorganic bases. When used in medicine the salts should be pharmaceutically acceptable, but non-pharmaceutically acceptable salts conveniently may be used to prepare pharmaceutically acceptable salts thereof. Such salts include, but are not limited to, those prepared from the following acids: hydrochloric, hydrobromic, sulphuric, nitric, phosphoric, maleic, acetic, salicylic, p-toluene sulphonic, tartaric, citric, methane sulphonic, formic, malonic, succinic, naphthalene-2-sulphonic, and benzene sulphonic. Also, such salts may be prepared as alkaline metal or alkaline earth salts, such as sodium, potassium or calcium salts of the carboxylic acid group.

By “pharmaceutically acceptable salt” is meant those salts which are, within the scope of sound medical judgment, Exemplary for use in contact with the tissues of humans and lower animals without undue toxicity, irritation, allergic response and the like and are commensurate with a reasonable benefit/risk ratio. Pharmaceutically acceptable salts are well-known in the art. For example, P. H. Stahl, et al. describe pharmaceutically acceptable salts in detail in “Handbook of Pharmaceutical Salts: Properties, Selection, and Use” (Wiley VCH, Zurich, Switzerland: 2002).

The salts may be prepared in situ during the final isolation and purification of the compounds described within the described invention or separately by reacting a free base function with an Exemplary organic acid. Representative acid addition salts include, but are not limited to, acetate, adipate, alginate, citrate, aspartate, benzoate, benzenesulfonate, bisulfate, butyrate, camphorate, camphorsufonate, digluconate, glycerophosphate, hemisulfate, heptanoate, hexanoate, fumarate, hydrochloride, hydrobromide, hydroiodide, 2-hydroxyethansulfonate(isethionate), lactate, maleate, methanesulfonate, nicotinate, 2-naphthalenesulfonate, oxalate, pamoate, pectinate, persulfate, 3-phenylpropionate, picrate, pivalate, propionate, succinate, tartrate, thiocyanate, phosphate, glutamate, bicarbonate, p-toluenesulfonate and undecanoate. Also, the basic nitrogen-containing groups may be quaternized with such agents as lower alkyl halides, such as methyl, ethyl, propyl, and butyl chlorides, bromides and iodides; dialkyl sulfates like dimethyl, diethyl, dibutyl and diamyl sulfates; long chain halides, such as decyl, lauryl, myristyl and stearyl chlorides, bromides and iodides; arylalkyl halides, such as benzyl and phenethyl bromides, and others. Water or oil-soluble or dispersible products are thereby obtained. Examples of acids which may be employed to form pharmaceutically acceptable acid addition salts include such inorganic acids as hydrochloric acid, hydrobromic acid, sulphuric acid and phosphoric acid and such organic acids as oxalic acid, maleic acid, succinic acid and citric acid. Basic addition salts may be prepared in situ during the final isolation and purification of compounds described within the invention by reacting a carboxylic acid-containing moiety with an Exemplary base such as the hydroxide, carbonate or bicarbonate of a pharmaceutically acceptable metal cation or with ammonia or an organic primary, secondary or tertiary amine. Pharmaceutically acceptable salts include, but are not limited to, cations based on alkali metals or alkaline earth metals such as lithium, sodium, potassium, calcium, magnesium and aluminum salts and the like and nontoxic quaternary ammonia and amine cations including ammonium, tetramethylammonium, tetraethylammonium, methylamine, dimethylamine, trimethylamine, triethylamine, diethylamine, ethylamine and the like. Other representative organic amines useful for the formation of base addition salts include ethylenediamine, ethanolamine, diethanolamine, piperidine, piperazine and the like. Pharmaceutically acceptable salts may be also obtained using standard procedures well known in the art, for example by reacting a sufficiently basic compound such as an amine with a Exemplary acid affording a physiologically acceptable anion. Alkali metal (for example, sodium, potassium or lithium) or alkaline earth metal (for example calcium or magnesium) salts of carboxylic acids may also be made.

The formulations may be presented conveniently in unit dosage form and may be prepared by any of the methods well known in the art of pharmacy. All methods include the step of bringing into association a composition, or a pharmaceutically acceptable salt or solvate thereof (“active compound”) with the carrier which constitutes one or more accessory agents. In general, the formulations are prepared by uniformly and intimately bringing into association the active agent with liquid carriers or finely divided solid carriers or both and then, if necessary, shaping the product into the desired formulation.

The pharmaceutical agent or a pharmaceutically acceptable ester, salt, solvate or prodrug thereof may be mixed with other active materials that do not impair the desired action, or with materials that supplement the desired action. Solutions or suspensions used for parenteral, intradermal, subcutaneous, intrathecal, or topical application may include, but are not limited to, for example, the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parenteral preparation may be enclosed in ampoules, disposable syringes or multiple dose vials made of glass or plastic. Administered intravenously, particular carriers are physiological saline or phosphate buffered saline (PBS).

Pharmaceutical compositions for parenteral injection comprise pharmaceutically acceptable sterile aqueous or nonaqueous solutions, dispersions, suspensions or emulsions and sterile powders for reconstitution into sterile injectable solutions or dispersions. Examples of Exemplary aqueous and nonaqueous carriers, diluents, solvents or vehicles include water, ethanol, polyols (propylene glycol, polyethylene glycol, glycerol, and the like), Exemplary mixtures thereof, vegetable oils (such as olive oil) and injectable organic esters such as ethyl oleate. Proper fluidity may be maintained, for example, by the use of a coating such as lecithin, by the maintenance of the required particle size in the case of dispersions, and by the use of surfactants.

These compositions also may contain adjuvants including preservative agents, wetting agents, emulsifying agents, and dispersing agents. Prevention of the action of microorganisms may be ensured by various antibacterial and antifungal agents, for example, parabens, chlorobutanol, phenol, sorbic acid, and the like. It also may be desirable to include isotonic agents, for example, sugars, sodium chloride and the like. Prolonged absorption of the injectable pharmaceutical form may be brought about by the use of agents delaying absorption, for example, aluminum monostearate and gelatin.

Suspensions, in addition to the active compounds, may contain suspending agents, as, for example, ethoxylated isostearyl alcohols, polyoxyethylene sorbitol and sorbitan esters, microcrystalline cellulose, aluminum metahydroxide, bentonite, agar-agar, tragacanth, and mixtures thereof.

The therapeutic agent(s), including the composition(s) of the described invention may be provided in particles. The term “particles” as used herein refers to nano or microparticles (or in some instances larger) that may contain in whole or in part the composition or the other therapeutic agent(s) as described herein. The particles may contain the therapeutic agent(s) in a core surrounded by a coating. The therapeutic agent(s) also may be dispersed throughout the particles. The therapeutic agent(s) also may be adsorbed into the particles. The particles may be of any order release kinetics, including zero order release, first order release, second order release, delayed release, sustained release, immediate release, etc., and any combination thereof. The particle may include, in addition to the therapeutic agent(s), any of those materials routinely used in the art of pharmacy and medicine, including, but not limited to, erodible, nonerodible, biodegradable, or nonbiodegradable material or combinations thereof. The particles may be microcapsules that contain the composition in a solution or in a semi-solid state. The particles may be of virtually any shape.

Both non-biodegradable and biodegradable polymeric materials may be used in the manufacture of particles for delivering the therapeutic agent(s). Such polymers may be natural or synthetic polymers. The polymer is selected based on the period of time over which release is desired. For example, bioadhesive polymers include bioerodible hydrogels as described by Sawhney et al in Macromolecules (1993) 26, 581-587, the teachings of which are incorporated herein by reference. These include polyhyaluronic acids, casein, gelatin, glutin, polyanhydrides, polyacrylic acid, alginate, chitosan, poly(methyl methacrylates), poly(ethyl methacrylates), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate).

The therapeutic agent(s) may be contained in controlled release systems. In order to prolong the effect of a drug, it often is desirable to slow the absorption of the drug from subcutaneous, intrathecal, or intramuscular injection. This may be accomplished by the use of a liquid suspension of crystalline or amorphous material with poor water solubility. The rate of absorption of the drug then depends upon its rate of dissolution which, in turn, may depend upon crystal size and crystalline form. The term “controlled release” is intended to refer to any drug-containing formulation in which the manner and profile of drug release from the formulation are controlled. This refers to immediate as well as non-immediate release formulations, with non-immediate release formulations including, but not limited to, sustained release and delayed release formulations. The term “sustained release” (also referred to as “extended release”) is used herein in its conventional sense to refer to a drug formulation that provides for gradual release of a drug over an extended period of time, and that can result in substantially constant blood levels of a drug over an extended time period. Alternatively, delayed absorption of a parenterally administered drug form is accomplished by dissolving or suspending the drug in an oil vehicle. The term “delayed release” is used herein in its conventional sense to refer to a drug formulation in which there is a time delay between administration of the formulation and the release of the drug there from. “Delayed release” may or may not involve gradual release of drug over an extended period of time, and thus may or may not be “sustained release.”

According to some embodiments, use of a long-term sustained release implant may be desirable for treatment of chronic conditions. The term “long-term” release, as used herein, means that the implant is constructed and arranged to deliver therapeutic levels of the active ingredient for at least 7 days, and preferably about 30 to about 60 days. Long-term sustained release implants are well-known to those of ordinary skill in the art and include some of the release systems described above.

Injectable depot forms are made by forming microencapsulated matrices of a described antibiotic in biodegradable polymers such as polylactide-polyglycolide. Depending upon the ratio of antibiotic to polymer and the nature of the particular polymer employed, the rate of drug release may be controlled. Such long acting formulations may be formulated with appropriate polymeric or hydrophobic materials (for example as an emulsion in an acceptable oil) or ion exchange resins, or as sparingly soluble derivatives, for example, as a sparingly soluble salt. Examples of other biodegradable polymers include poly(orthoesters) and poly(anhydrides). Depot injectable formulations also are prepared by entrapping the antibiotic of the described invention in liposomes or microemulsions, which are compatible with body tissues.

The injectable formulations may be sterilized, for example, by filtration through a bacterial-retaining filter or by incorporating sterilizing agents in the form of sterile solid compositions that may be dissolved or dispersed in sterile water or other sterile injectable medium just prior to use. Injectable preparations, for example, sterile injectable aqueous or oleaginous suspensions may be formulated according to the known art using suitable dispersing or wetting agents and suspending agents. The sterile injectable preparation also may be a sterile injectable solution, suspension or emulsion in a nontoxic, parenterally acceptable diluent or solvent such as a solution in 1,3-butanediol. Among the acceptable vehicles and solvents that may be employed are water, Ringer's solution, U.S.P. and isotonic sodium chloride solution. In addition, sterile, fixed oils conventionally are employed or as a solvent or suspending medium. For this purpose any bland fixed oil may be employed including synthetic mono- or diglycerides. In addition, fatty acids such as oleic acid are used in the preparation of injectables.

Formulations for parenteral (including but not limited to, subcutaneous, intradermal, intramuscular, intravenous, intrathecal and intraarticular) administration include aqueous and non-aqueous sterile injection solutions that may contain anti-oxidants, buffers, bacteriostats and solutes, which render the formulation isotonic with the blood of the intended recipient; and aqueous and non-aqueous sterile suspensions, which may include suspending agents and thickening agents. The formulations may be presented in unit-dose or multi-dose containers, for example sealed ampules and vials, and may be stored in a freeze-dried (lyophilized) condition requiring only the addition of the sterile liquid carrier, for example, saline, water-for-injection, immediately prior to use. Extemporaneous injection solutions and suspensions may be prepared from sterile powders, granules and tablets of the kind previously described.

Exemplary buffering agents include: acetic acid and a salt (1-2% w/v); citric acid and a salt (1-3% w/v); boric acid and a salt (0.5-2.5% w/v); and phosphoric acid and a salt (0.8-2% w/v). Exemplary preservatives include benzalkonium chloride (0.003-0.03% w/v); chlorobutanol (0.3-0.9% w/v); parabens (0.01-0.25% w/v) and thimerosal (0.004-0.02% w/v).

For oral administration in the form of tablets or capsules, the active drug component may be combined with any oral non-toxic pharmaceutically acceptable inert carrier, such as lactose, starch, sucrose, cellulose, magnesium stearate, dicalcium phosphate, calcium sulfate, talc, mannitol, ethyl alcohol (liquid forms) and the like. Moreover, when desired or needed, suitable binders, lubricants, disintegrating agents and coloring agents also may be incorporated in the mixture. Powders and tablets may be comprised of from about 5 to about 95 percent of the described composition. Exemplary binders include starch, gelatin, natural sugars, corn sweeteners, natural and synthetic gums such as acacia, sodium alginate, carboxymethylcellulose, polyethylene glycol and waxes. Among the lubricants there may be mentioned for use in these dosage forms, boric acid, sodium benzoate, sodium acetate, sodium chloride, and the like. Disintegrants include starch, methylcellulose, guar gum and the like. Sweetening and flavoring agents and preservatives may also be included where appropriate.

The compositions of the invention also may be formulated as syrups and elixirs. Syrups and elixirs may be formulated with sweetening agents, for example, glycerol, propylene glycol, sorbitol or sucrose. Such formulations also may contain a demulcent, a preservative, and flavoring and coloring agents. Demulcents are protective agents employed primarily to alleviate irritation, particularly mucous membranes or abraded tissues. A number of chemical substances possess demulcent properties. These substances include the alginates, mucilages, gums, dextrins, starches, certain sugars, and polymeric polyhydric glycols. Others include acacia, agar, benzoin, carbomer, gelatin, glycerin, hydroxyethyl cellulose, hydroxypropyl cellulose, hydroxypropyl methylcellulose, propylene glycol, sodium alginate, tragacanth, hydrogels and the like.

For buccal administration, the compositions of the present invention may take the form of tablets or lozenges formulated in a conventional manner for this route.

Liquid form preparations include solutions, suspensions and emulsions.

Liquid form preparations also may include solutions for intranasal administration.

The compositions of the present invention may be in the form of a dispersible dry powder for delivery by inhalation or insufflation (either through the mouth or through the nose). Dry powder compositions may be prepared by processes known in the art, such as lyophilization and jet milling, as disclosed in International Patent Publication No. WO 91/16038 and as disclosed in U.S. Pat. No. 6,921,527, the disclosures of which are incorporated by reference. The composition of the present invention is placed within a dosage receptacle in an amount sufficient to provide a subject with a unit dosage treatment. The dosage receptacle is one that fits within an inhalation device to allow for the aerosolization of the dry powder composition by dispersion into a gas stream to form an aerosol and then capturing the aerosol so produced in a chamber having a mouthpiece attached for subsequent inhalation by a subject in need of treatment. Such a dosage receptacle includes any container enclosing the composition known in the art such as gelatin or plastic capsules with a removable portion that allows a stream of gas (e.g., air) to be directed into the container to disperse the dry powder composition. Such containers are exemplified by those shown in U.S. Pat. No. 4,227,522; U.S. Pat. No. 4,192,309; and U.S. Pat. No. 4,105,027. Exemplary containers also include those used in conjunction with Glaxo's Ventolin® Rotohaler brand powder inhaler or Fison's Spinhaler® brand powder inhaler. Another Exemplary unit-dose container which provides a superior moisture barrier is formed from an aluminum foil plastic laminate. The pharmaceutical-based powder is filled by weight or by volume into the depression in the formable foil and hermetically sealed with a covering foil-plastic laminate. Such a container for use with a powder inhalation device is described in U.S. Pat. No. 4,778,054 and is used with Glaxo's Diskhaler® (U.S. Pat. Nos. 4,627,432; 4,811,731; and 5,035,237). Each of these references is incorporated herein by reference.

The compositions of the present invention may be in the form of suppositories for rectal administration of the composition. “Rectal” or “rectally” as used herein refers to introduction into the body through the rectum where absorption occurs through the walls of the rectum. These compositions can be prepared by mixing the drug with a Exemplary nonirritating excipient such as cocoa butter and polyethylene glycols which are solid at ordinary temperatures but liquid at the rectal temperature and will therefore melt in the rectum and release the drug. When formulated as a suppository the compositions of the invention may be formulated with traditional binders and carriers, such as triglycerides.

The term “topical” refers to administration of an inventive composition at, or immediately beneath, the point of application. The phrase “topically applying” describes application onto one or more surfaces(s) including epithelial surfaces. Ttopical administrationgenerally provides a local rather than a systemic effect For the purpose of this application, topical applications shall include mouthwashes and gargles.

Topical administration may also involve the use of transdermal administration such as transdermal patches or iontophoresis devices which are prepared according to techniques and procedures well known in the art. The terms “transdermal delivery system”, transdermal patch” or “patch” refer to an adhesive system placed on the skin to deliver a time released dose of a drug(s) by passage from the dosage form through the skin to be available for distribution via the systemic circulation. Transdermal patches are a well-accepted technology used to deliver a wide variety of pharmaceuticals, including, but not limited to, scopolamine for motion sickness, nitroglycerin for treatment of angina pectoris, clonidine for hypertension, estradiol for post-menopausal indications, and nicotine for smoking cessation.

Patches for use in the present invention include, but are not limited to, (1) the matrix patch; (2) the reservoir patch; (3) the multi-laminate drug-in-adhesive patch; and (4) the monolithic drug-in-adhesive patch; TRANSDERMAL AND TOPICAL DRUG DELIVERY SYSTEMS, pp. 249-297 (Tapash K. Ghosh et al. eds., 1997), hereby incorporated herein by reference. These patches are well known in the art and generally available commercially.

Unless defined otherwise, all technical and scientific terms used herein have the same meaning as commonly understood by one of ordinary skill in the art to which this invention belongs. Although any methods and materials similar or equivalent to those described herein also can be used in the practice or testing of the described invention, the preferred methods and materials are now described. All publications mentioned herein are incorporated herein by reference to disclose and describe the methods and/or materials in connection with which the publications are cited.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges which may independently be included in the smaller ranges is also encompassed within the invention, subject to any specifically excluded limit in the stated range. Where the stated range includes one or both of the limits, ranges excluding either both of those included limits are also included in the invention.

It must be noted that as used herein and in the appended claims, the singular forms “a”, “and”, and “the” include plural references unless the context clearly dictates otherwise. All technical and scientific terms used herein have the same meaning.

The publications discussed herein are provided solely for their disclosure prior to the filing date of the present application. Nothing herein is to be construed as an admission that the described invention is not entitled to antedate such publication by virtue of prior invention. Further, the dates of publication provided may be different from the actual publication dates which may need to be independently confirmed.

### EXAMPLES

The following examples are put forth so as to provide those of ordinary skill in the art with a complete disclosure and description of how to make and use the described invention, and are not intended to limit the scope of what the inventors regard as their invention nor are they intended to represent that the experiments below are all or the only experiments performed. Efforts have been made to ensure accuracy with respect to numbers used (e.g., amounts, temperatures, etc.) but some experimental errors and deviations should be accounted for. Unless indicated otherwise, parts are by weight, molecular weight is weight average molecular weight, temperature is in degrees Centigrade, and pressure is at or near atmospheric.

### Example 1

### Possible Structures for Biologically Active Mangrolide A Isolated from Marine Actinomycete

Before the total synthesis work described below, the configuration of the C8-C9 double bond; absolute configuration at C10 and C11 and absolute configuration of the sugars in natural Mangrolide A was unknown.

Hydrolysis of natural mangrolide A with methanol (MeOH), H+ provides a mangrolide Aglycon methyl ether with a C8-C9 Z double bond or C8-C9 E double bond.

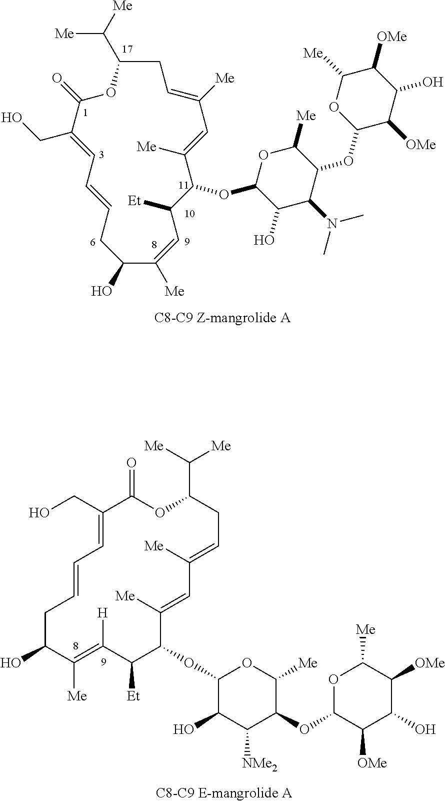

In order to fully assign the structure of mangrolide A (including double bond geometry and absolute configuration at C10 and C11, we prepared the following three possible structures of mangrolide A methylether aglycon:

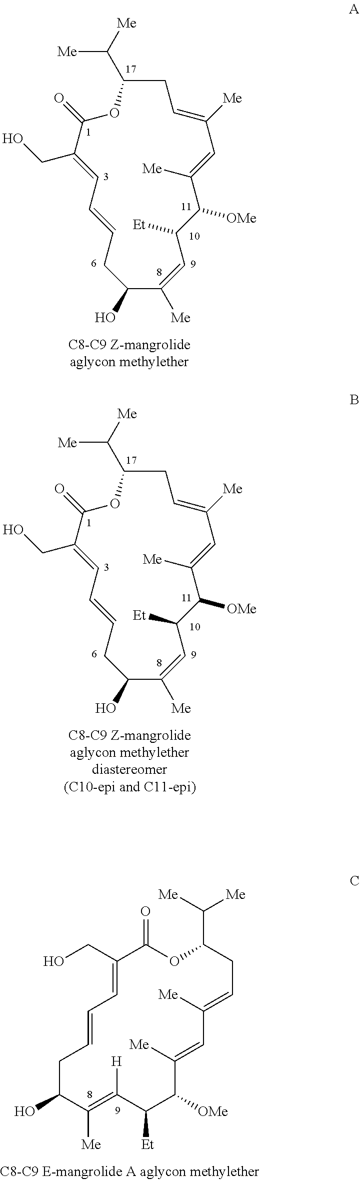 [SMILES: CC[C@H]1/C=C(/C)[C@@H](O)C/C=C/C=C(\CO)C(=O)O[C@H](C(C)C)C/C=C(C)/C=C(\C)[C@@H]1OC ; CC[C@@H]1/C=C(/C)[C@@H](O)C/C=C/C=C(\CO)C(=O)O[C@H](C(C)C)C/C=C(C)/C=C(\C)[C@H]1OC ; CC[C@H]1/C=C(\C)[C@@H](O)C/C=C/C=C(\CO)C(=O)O[C@H](C(C)C)C/C=C(C)/C=C(\C)[C@@H]1OC]

As will be demonstrated in the Examples that follow, we concluded that the mangrolide Aglycon (and hence natural mangrolide A) has the structure with double bond geometry and relative/absolute configuration as drawn/represented in structure C.

### Example 2

### Synthesis of Mangrolide Antibacterial Agent

Scheme 1.

The following synthetic scheme illustrates synthesis of a fragment common to all proposed possible structures A, B, and C of Formula 1.6:

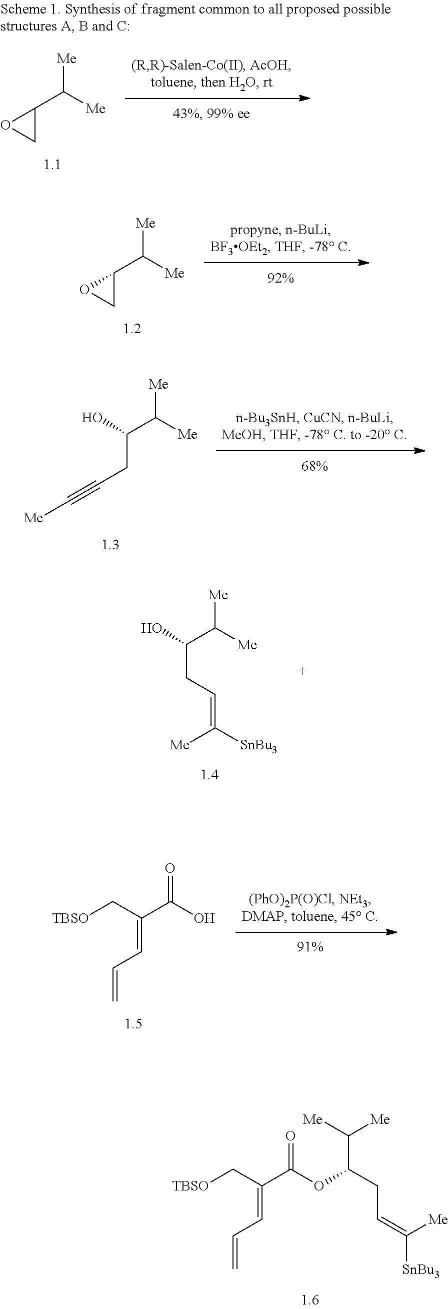

According to one embodiment, a method for the synthesis of a fragment of Formula 1.6

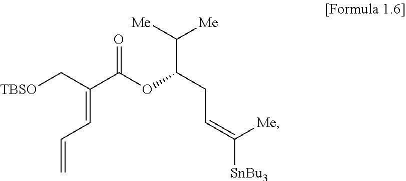

or a pharmaceutically acceptable salt thereof comprises, in order:

(a) reacting a compound of Formula 1.1:

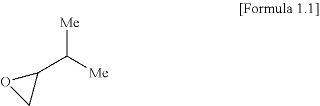

with acetic acid, toluence in the presence of a (R, R)-Salen-Co (II) catalyst at room temperature to form a compound of Formula 1.2:

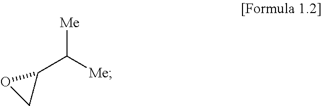

(b) reacting the compound of formula 1.2 in the presence of 1-propyne, n-butyllithium (n-BuLi) boron trifluoride diethyl etherate (BF₃OEt₂), and tetrahydrofuran (THF) at −78° C. to form a compound of Formula 1.3:

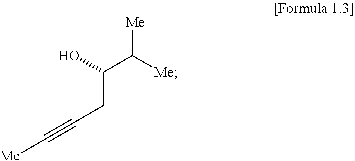

(c) reacting the compound of formula 1.3 in the presence of tributyl tin hydride (Bu₃SnH, which catalyzes the deoxygenation of alcohols), copper (I) cyanide (CuCN), n-butyllithium (n-BuLi), methanol, and tetrahydrofuran (THF), at −78° C. to −20° C., to form a compound of Formula 1.4,

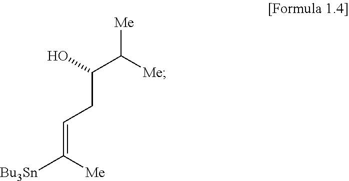

and
(d) reacting the compound of formula 1.4 comprising an SnBu₃ protecting group, and a compound of Formula 1.5 comprising a tert-butyl dimethyl silyl (TBS) protecting group,

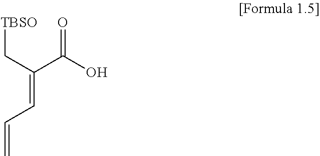 [SMILES: C=C/C=C(\CO[Si](C)(C)C(C)(C)C)C(=O)O]

in the presence of diphenyl phosphate chlorate (PhO)₂P(O)Cl), triethylamine (NEt₃), and 4-dimethyl aminopyridine (DMAP), a nucleophilic catalyst for esterification, in toluene at 45° C. to form a compound of Formula 1.6:

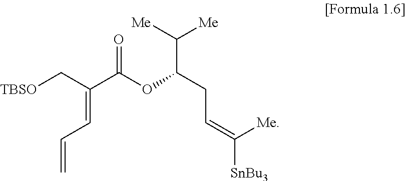

Scheme 2.

Synthesis of fragment required to synthesize possible mangrolide A glycon methylether isomer A.

The following synthetic scheme illustrates the generation of a fragment required to synthesize a possible mangrolide aglycon methylether isomer A of Formula 2.13:

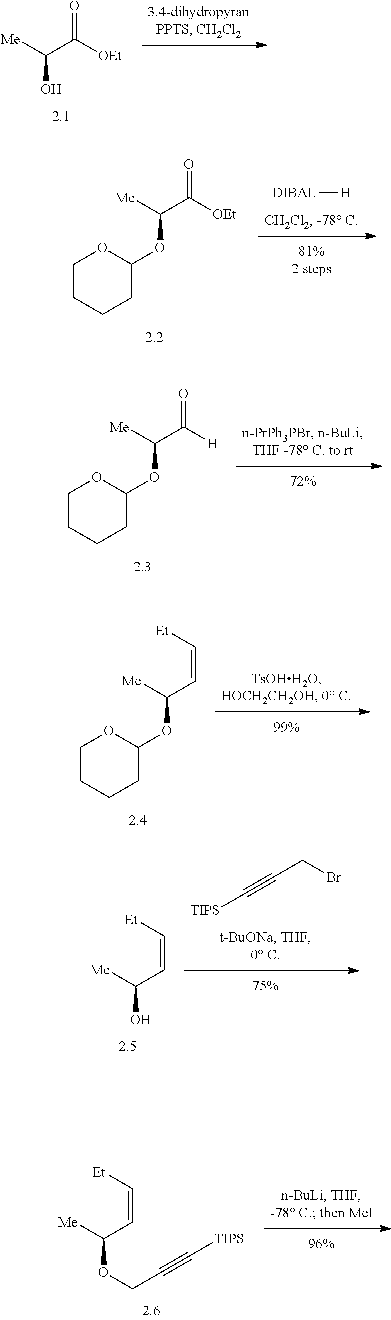

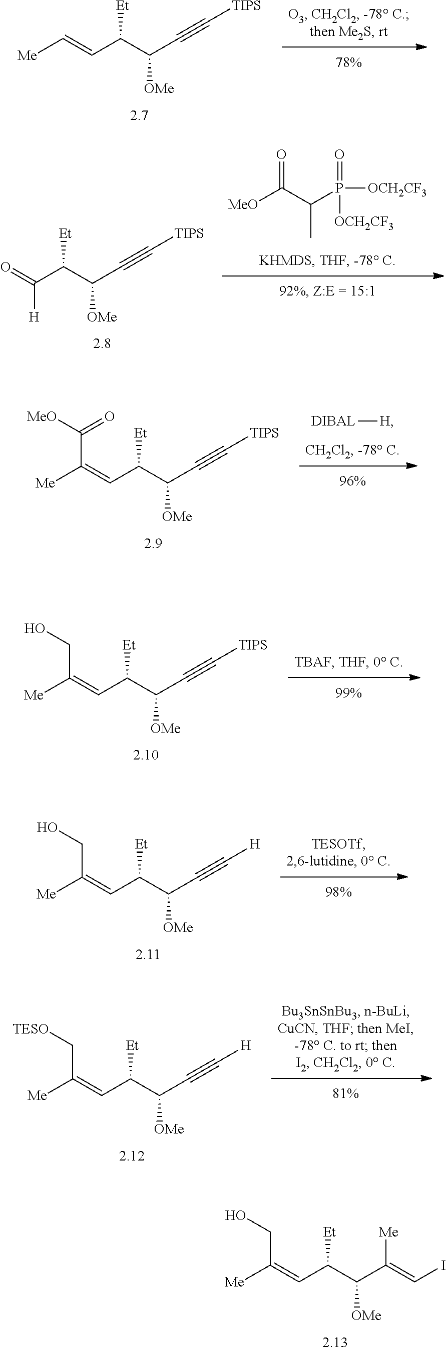

According to one embodiment, a method for the synthesis of a compound of Formula 2.13:

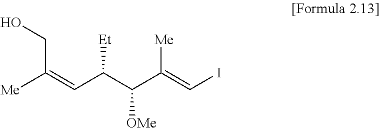 [SMILES: CC[C@@H](/C=C(/C)CO)[C@@H](OC)/C(C)=C/I]

comprises, in order

(a) reacting a compound of Formula 2.1:

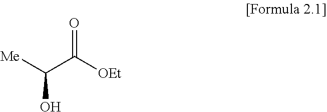

in the presence of 3,4-dihydropyran, pyridinium p-toluene sulfonate (PPTS), and dichloromethane (CH₂Cl₂) to form a compound of formula 2.2

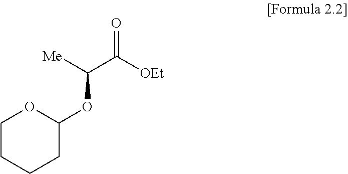 [SMILES: CCOC(=O)[C@H](C)OC1CCCCO1]

(b) reacting the compound of Formula 2.2 in the presence of Diisobutyl aluminum hydride (DIBAL) and dichloromethane (DCM) at −78° C. to form a compound of Formula 2.3:

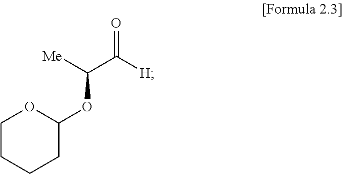

(c) reacting the compound of Formula 2.3 with n-propyl triphenyl phosphonium bromide (nPrPh₃PBr), n-butyllithium (nBuLi), and tetrahydrofuran (THF) at −78° C. to room temperature to form a compound of Formula 2.4:

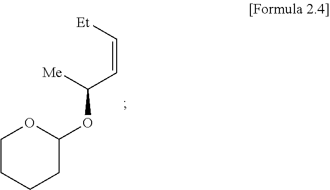 [SMILES: CC/C=C\[C@H](C)OC1CCCCO1]

(d) reacting the compound of Formula 2.4 with tosylic acid (CH₃C₆H₄SO₃H, TsOH), and ethylene glycol (HO(CH₂)₂OH) at 0° C. to form a compound of Formula 2.5:

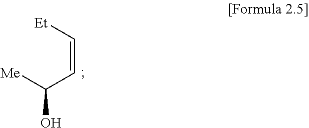

(e) reacting the compound of Formula 2.5 with a compound of Formula J, wherein TIPS signifies a triisopropylsilyl ether protecting group:

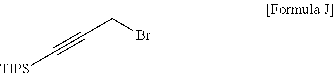

(in the presence of sodium tert butoxide (tBuONa) and tetrahydrofuran (THF) at 0° C. to form a compound of Formula 2.6:

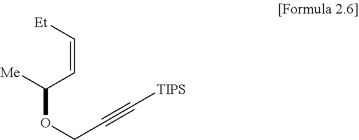

(f) reacting the compound of Formula 2.6 in the presence of (i) n-butyllithium (n-BuLi), tetrahydrofuran (THF) at −78° C., and then methyl iodide (MeI) to form a compound of formula 2.7:

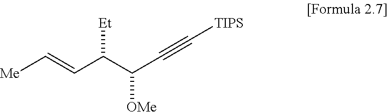

(g) reacting the compound of Formula 2.7 in the presence of (i) ozone (O₃), dichloromethane (CH₂Cl₂) at −78° C. and then dimethyl sulfide (Me₂S) at room temperature to form a compound of Formula 2.8:

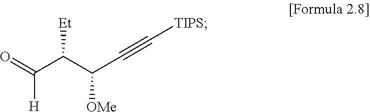

(h) reacting the compound of Formula 2.8 in the presence of (i)

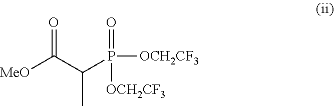

potassium bis (trimethylsilyl)amide (KHMDS) and tetrahydrofuran (THF) at −78° C. to form a compound of Formula 2.9:

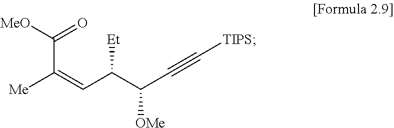

and

(i) reacting the compound of Formula 2.9 in the presence of (i) diisobutylaluminum hydride (DIBAL) at −78° C. and dichloromethane (CH₂Cl₂); to form a compound of Formula 2; 10:

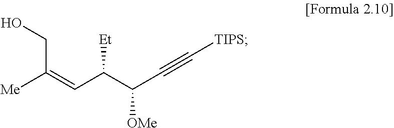

(j) reacting the compound of Formula 2.10 in the presence of tetra-n-butylammonium fluoride (TBAF) and tetrahydrofuran (THF) at 0° C. to form a compound of Formula 2.11:

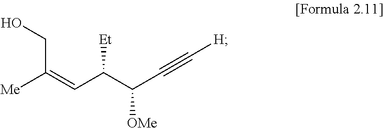

(j) reacting the compound of Formula 2.11 in the presence of triethylsilyltrifluoromethane sulfonate (TESOTf) and 2,6-lutidine, at 0° C. to form a compound of Formula 2.12:

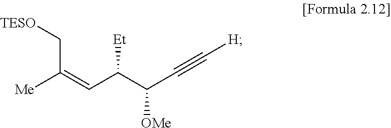

and

(k) reacting the compound of Formula 2.12 in the presence of tributyl tin hydride (Bu₃SnSnBu₃), cupper cyanide (CuCN), tetrahydrofuran (THF), then methyl iodide at −78° C. to room temperature; then elemental iodine (I₂); and dichloromethane (CH₂Cl₂) at 0° C. to form a compound of Formula 2.13:

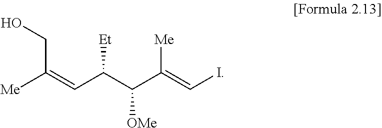 [SMILES: CC[C@@H](/C=C(/C)CO)[C@@H](OC)/C(C)=C/I]

Scheme 3.

The following synthetic scheme illustrates the synthesis of a compound of Formula 3.13 required to synthesize a possible mangrolide aglycon methylether isomer B:

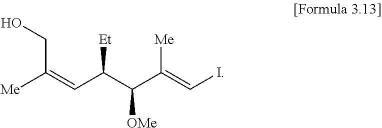 [SMILES: CC[C@H](/C=C(/C)CO)[C@H](OC)/C(C)=C/I]

Note that the compound of Formula 3.13 is the mirror image of the compound of Formula 2.13 in Scheme 2.

Synthetic Scheme 3. Synthesis of Fragment Required to Synthesize Possible Mangrolide Aglycon Methylether Isomer B

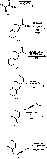

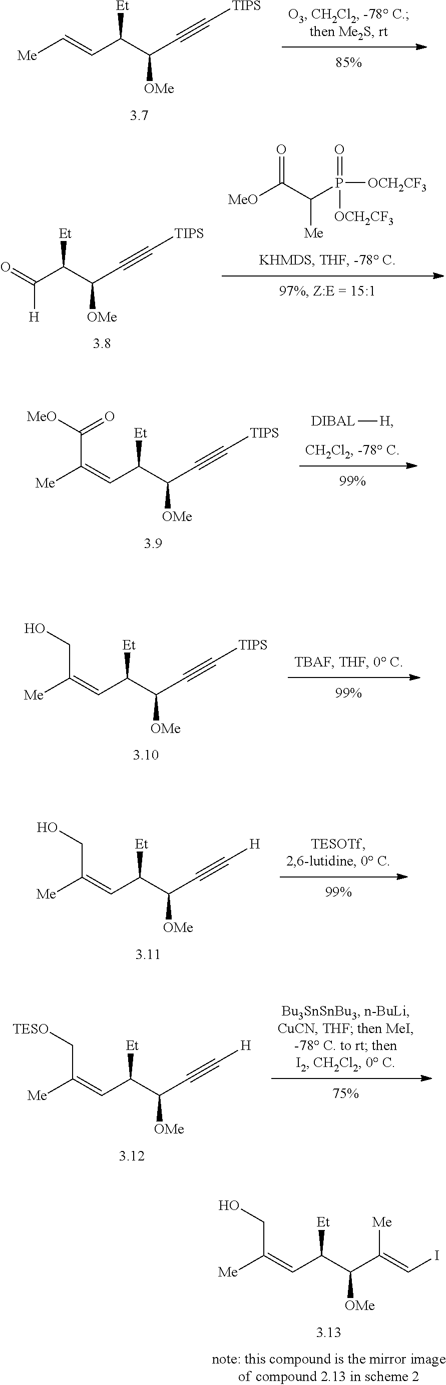

According to one embodiment, a method for the synthesis of a compound of Formula 3.13:

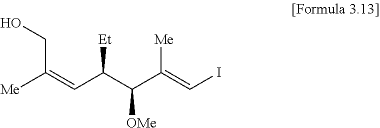 [SMILES: CC[C@H](/C=C(/C)CO)[C@H](OC)/C(C)=C/I]

comprises, in order

(a) reacting a compound of Formula 3.1:

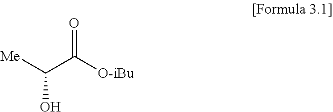

in the presence of 3,4-dihydropyran, pyridinium p-toluene sulfonate (PPTS), and dichloromethane (CH₂Cl₂) to form a compound of Formula 3.2

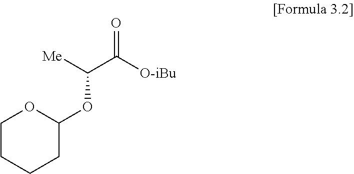

(b) reacting the compound of Formula 3.2 in the presence of Diisobutyl aluminum hydride (DIBAL) and dichloromethane (DCM) at −78° C. to form a compound of Formula 3.3:

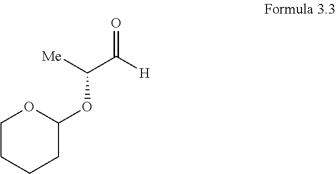 [SMILES: C[C@H](C=O)OC1CCCCO1]

(c) reacting the compound of Formula 2.3 with n-propyl triphenyl phosphobromide (nPrPh₃PBr), n-butyllithium (nBuLi), and tetrahydrofuran (THF) at −78° C. to room temperature to form a compound of Formula 3.4:

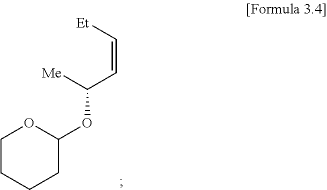 [SMILES: CC/C=C\[C@@H](C)OC1CCCCO1]

(d) reacting the compound of Formula 3.4 with, tosylic acid (CH₃C₆H₄SO₃H, TsOH), and ethylene glycol (HO(CH₂)₂OH) at 0° C. to form a compound of Formula 3.5:

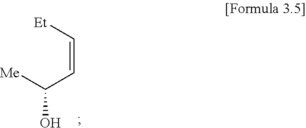

(e) reacting the compound of Formula 3.5 with a compound of Formula J, wherein TIPS signifies a triisopropylsilyl ether protecting group:

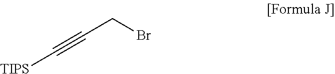

(in the presence of sodium tert butoxide (tBuONa) and tetrahydrofuran (THF) at 0° C. to form a compound of Formula 3.6:

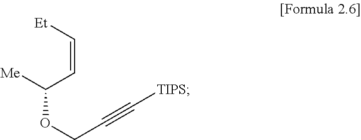

(f) reacting the compound of Formula 3.6 in the presence of (i) n-butyllithium (n-BuLi), tetrahydrofuran (THF) at −78° C., and then methyl iodide (MeI) to form a compound of Formula 3.7:

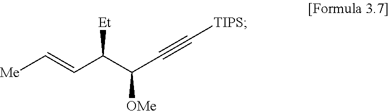

(g) reacting the compound of Formula 3.7 in the presence of (i) ozone (O₃), dichloromethane (CH₂Cl₂) at −78° C. and then dimethyl sulfide (Me₂S) at room temperature to form a compound of Formula 3.8:

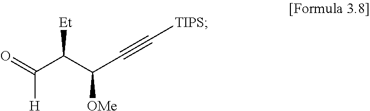

(h) reacting the compound of Formula 3.8 in the presence of (i)

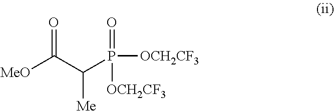

potassium bis (trimethylsilyl)amide (KHMDS) and tetrahydrofuran (THF) at −78° C. to form a compound of Formula 3.9:

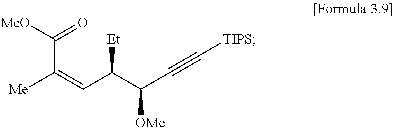

(i) reacting the compound of Formula 3.9 in the presence of (i) diisobutylalluminum hydride (DIBAL) and dichloromethane (CH₂Cl₂) at −78° C.; to form a compound of Formula 3.10:

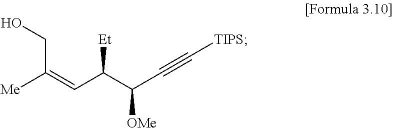

(j) reacting the compound of Formula 2.10 in the presence of tetra-n-butylammonium fluoride (TBAF) and tetrahydrofuran (THF) at 0° C. to form a compound of Formula 3.11:

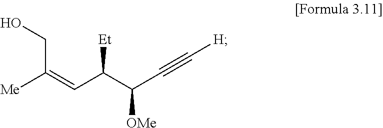

(j) reacting the compound of Formula 3.11 in the presence of triethylsilyltrifluoromethane sulfonate (TESOTf) and 2,6-lutidine, at 0° C. to form a compound of Formula 3.12:

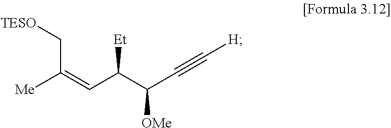

and

(k) reacting the compound of Formula 3.12 in the presence of tributyl tin hydride (Bu₃SnSnBu₃), copper cyanide (CuCN), tetrahydrofuran (THF), then methyl iodide at −78° C. to room temperature; then elemental iodine (I₂); and dichloromethane (CH₂Cl₂) at 0° C. to form the compound of Formula 3.13:

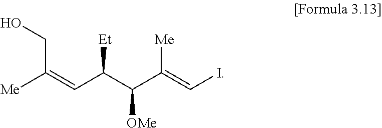 [SMILES: CC[C@H](/C=C(/C)CO)[C@H](OC)/C(C)=C/I]

Scheme 4.

The following synthetic scheme illustrates the synthesis of mangrolide Aglycon methylether possible structure A.

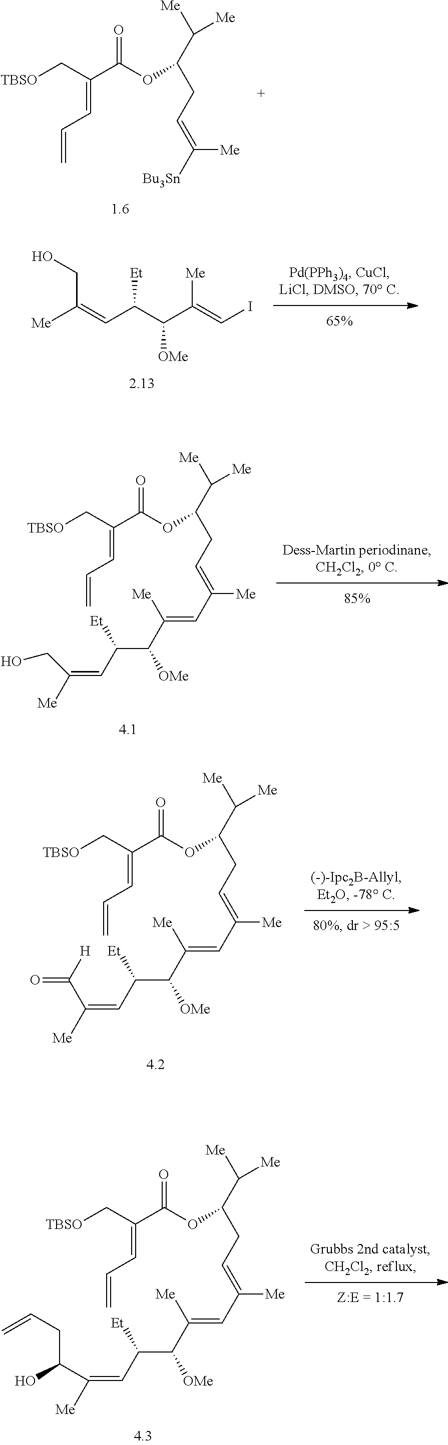

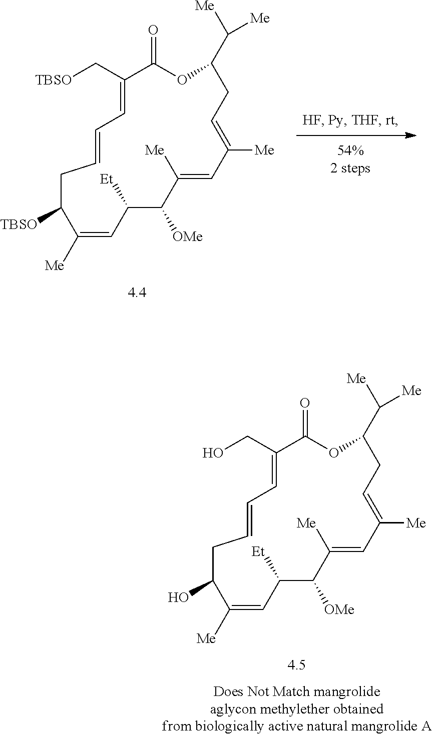 [SMILES: CC[C@H]1/C=C(/C)[C@@H](O[Si](C)(C)C(C)(C)C)C/C=C/C=C(\CO[Si](C)(C)C(C)(C)C)C(=O)O[C@H](C(C)C)C/C=C(C)/C=C(\C)[C@@H]1OC ; CC[C@H]1/C=C(/C)[C@@H](O)C/C=C/C=C(\CO)C(=O)O[C@H](C(C)C)C/C=C(C)/C=C(\C)[C@@H]1OC]

According to one embodiment, a method for the synthesis of mangrolide methyl ether of Formula 4.5:

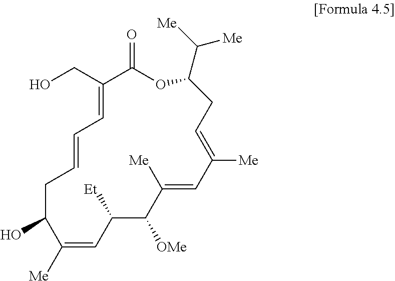 [SMILES: CC[C@H]1/C=C(/C)[C@@H](O)C/C=C/C=C(\CO)C(=O)O[C@H](C(C)C)C/C=C(C)/C=C(\C)[C@@H]1OC]

comprises, in order:

(a) reacting a compound of Formula 1.6:

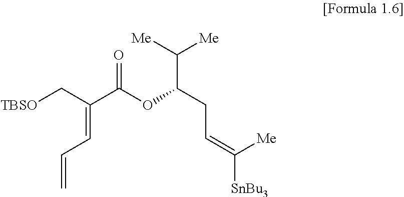

with a compound of Formula 2.13

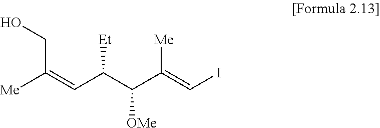 [SMILES: CC[C@@H](/C=C(/C)CO)[C@@H](OC)/C(C)=C/I]

in the presence of tetrakis(triphenylphosphine) palladium (Pd(PPh₃)₄, copper chloride (CuCl), lithium chloride (LiCl), and dimethylsulfoxide (DMSO) at 70° C. to form a compound of Formula 4.1:

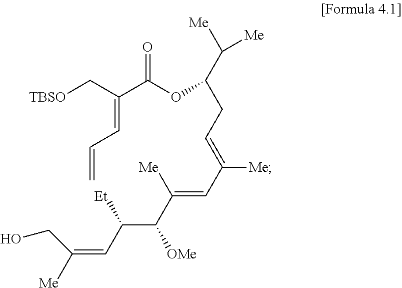 [SMILES: C=C/C=C(\CO[Si](C)(C)C(C)(C)C)C(=O)O[C@@H](C/C=C(C)/C=C(\C)[C@H](OC)[C@H](/C=C(/C)CO)CC)C(C)C]

(b) reacting a compound of Formula 4.1, in the presence of Dess-Martin periodinane and dichloromethane (CH₂Cl₂) at 0° C. to form a compound of Formula 4.2:

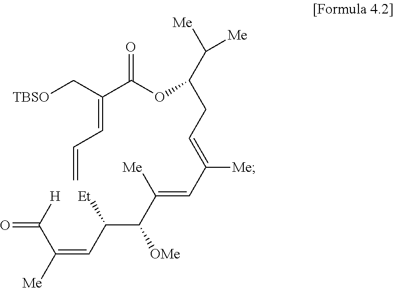 [SMILES: C=C/C=C(\CO[Si](C)(C)C(C)(C)C)C(=O)O[C@@H](C/C=C(C)/C=C(\C)[C@H](OC)[C@H](/C=C(/C)C=O)CC)C(C)C]

(c) reacting the compound of Formula 4.2 in the presence of (−)-lpc₂B-allyl, diethylether (Et₂O) at −78° C. to form a compound of Formula 4.3:

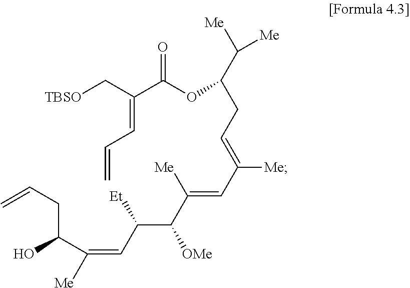 [SMILES: C=C/C=C(\CO[Si](C)(C)C(C)(C)C)C(=O)O[C@@H](C/C=C(C)/C=C(\C)[C@H](OC)[C@H](/C=C(/C)[C@@H](O)CC=C)CC)C(C)C]

(d) reacting the compound of Formula 4.3 in the presence of Grubbs 2^{nd} catalyst, dichloromethane (CH2Cl2) with reflux, to form a compound Formula 4.4:

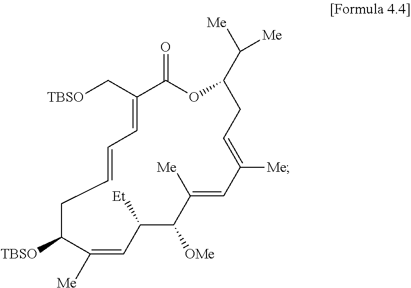 [SMILES: CC[C@H]1/C=C(/C)[C@@H](O[Si](C)(C)C(C)(C)C)C/C=C/C=C(\CO[Si](C)(C)C(C)(C)C)C(=O)O[C@H](C(C)C)C/C=C(C)/C=C(\C)[C@@H]1OC]

and

(e) reacting a compound of Formula 4.4 with hydrofluoric acid (HF), pyridine (PY); tetrahydrofuran (THF) at room temperature to form the compound of Formula 4.5:

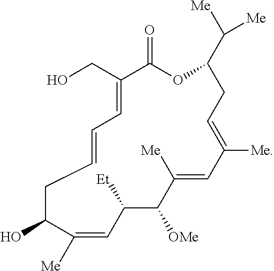 [SMILES: CC[C@H]1/C=C(/C)[C@@H](O)C/C=C/C=C(\CO)C(=O)O[C@H](C(C)C)C/C=C(C)/C=C(\C)[C@@H]1OC]

The compound of formula 4.5 does not match mangrolide Aglycon methylether obtained from biologically active natural mangrolide A.

Scheme 5.

The following synthetic scheme illustrates the synthesis of mangrolide Aglycon methylether of possible structure B.

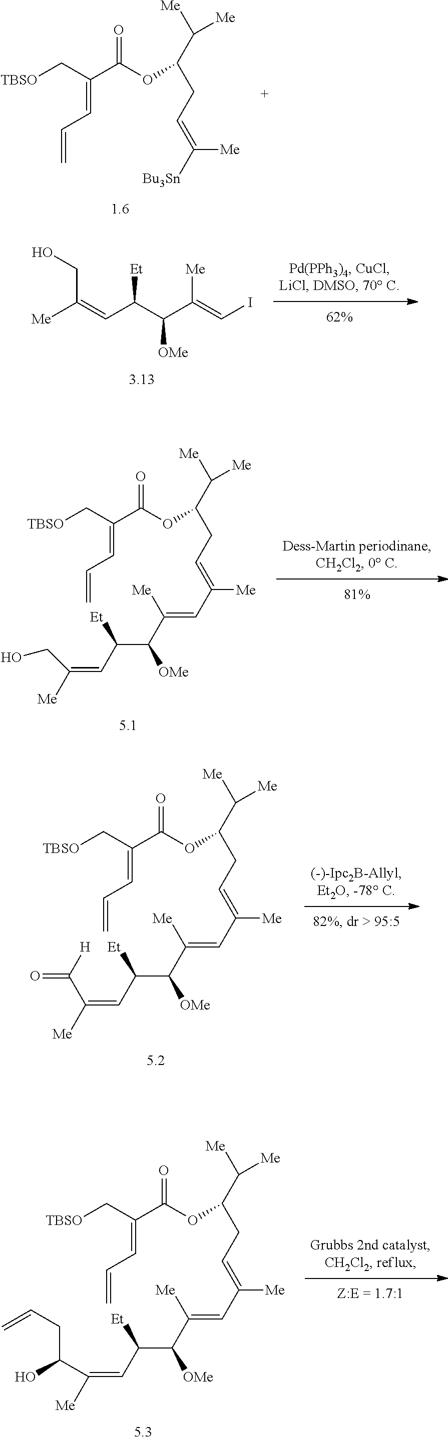

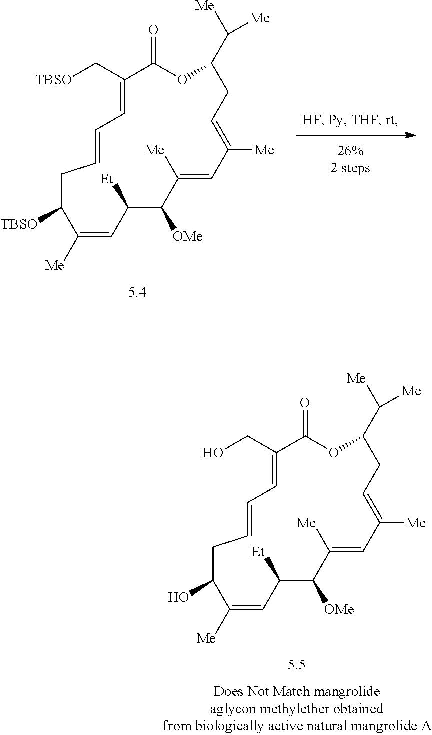 [SMILES: CC[C@@H]1/C=C(/C)[C@@H](O[Si](C)(C)C(C)(C)C)C/C=C/C=C(\CO[Si](C)(C)C(C)(C)C)C(=O)O[C@H](C(C)C)C/C=C(C)/C=C(\C)[C@H]1OC ; CC[C@@H]1/C=C(/C)[C@@H](O)C/C=C/C=C(\CO)C(=O)O[C@H](C(C)C)C/C=C(C)/C=C(\C)[C@H]1OC]

According to one embodiment, a method for the synthesis of a compound of Formula 5.5:

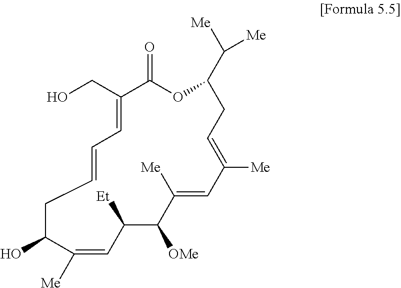 [SMILES: CC[C@@H]1/C=C(/C)[C@@H](O)C/C=C/C=C(\CO)C(=O)O[C@H](C(C)C)C/C=C(C)/C=C(\C)[C@H]1OC]

comprises, in order:

(a) reacting a compound of Formula 1.6

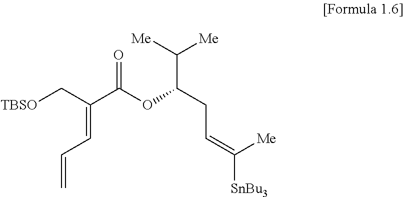

with a compound of Formula 3.13:

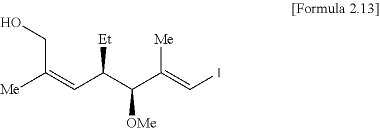 [SMILES: CC[C@H](/C=C(/C)CO)[C@H](OC)/C(C)=C/I]

in the presence of tetrakis(triphenylphosphine) palladium (Pd(PPh₃)₄, copper chloride (CuCl), lithium chloride (LiCl), and dimethylsulfoxide (DMSO) at 70° C. to form a compound of Formula 5.1:

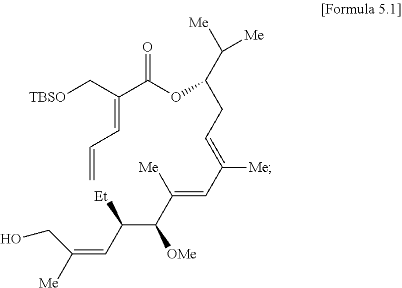 [SMILES: C=C/C=C(\CO[Si](C)(C)C(C)(C)C)C(=O)O[C@@H](C/C=C(C)/C=C(\C)[C@@H](OC)[C@@H](/C=C(/C)CO)CC)C(C)C]

(b) reacting a compound of Formula 5.1, in the presence of Dess-Martin periodinane and dichloromethane (CH₂Cl₂) at 0° C. to form a compound of Formula 5.2:

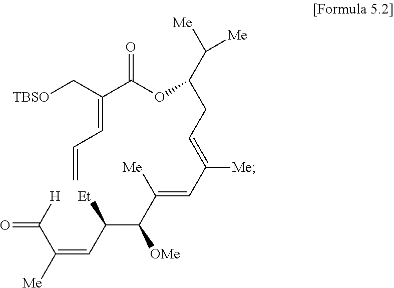 [SMILES: C=C/C=C(\CO[Si](C)(C)C(C)(C)C)C(=O)O[C@@H](C/C=C(C)/C=C(\C)[C@@H](OC)[C@@H](/C=C(/C)C=O)CC)C(C)C]

(c) reacting the compound of Formula 5.2 in the presence of (−)-lpc₂B-allyl, diethylether (Et₂O) at −78° C. to form a compound of Formula 5.3:

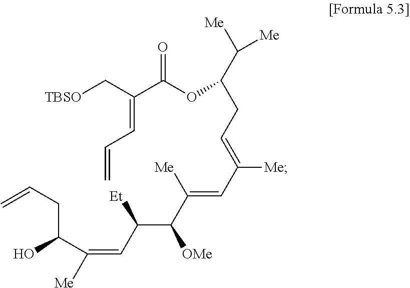 [SMILES: C=C/C=C(\CO[Si](C)(C)C(C)(C)C)C(=O)O[C@@H](C/C=C(C)/C=C(\C)[C@@H](OC)[C@@H](/C=C(/C)[C@@H](O)CC=C)CC)C(C)C]

(d) reacting the compound of Formula 5.3 in the presence of Grubbs 2^{nd} catalyst, dichloromethane (CH₂Cl₂) with reflux, to form a compound of Formula 5.4:

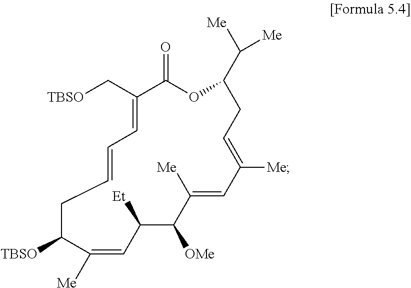 [SMILES: CC[C@@H]1/C=C(/C)[C@@H](O[Si](C)(C)C(C)(C)C)C/C=C/C=C(\CO[Si](C)(C)C(C)(C)C)C(=O)O[C@H](C(C)C)C/C=C(C)/C=C(\C)[C@H]1OC]

and

(e) reacting a compound of Formula 5.4 with hydrofluoric acid (HF), pyridine (PY); tetrahydrofuran (THF) at room temperature to form the compound of Formula 5.5:

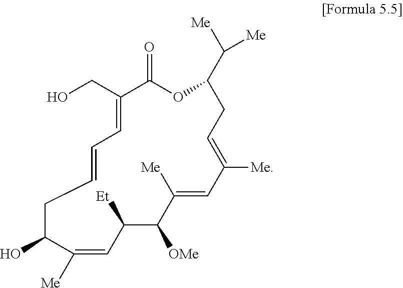 [SMILES: CC[C@@H]1/C=C(/C)[C@@H](O)C/C=C/C=C(\CO)C(=O)O[C@H](C(C)C)C/C=C(C)/C=C(\C)[C@H]1OC]

The compound of Formula 4.5 does not match mangrolide Aglycon methylether obtained from biologically active natural mangrolide A.

Scheme 6.

The following synthetic scheme shows synthesis of a fragment required to synthesize possible mangrolide Aglycon methylether isomer C.

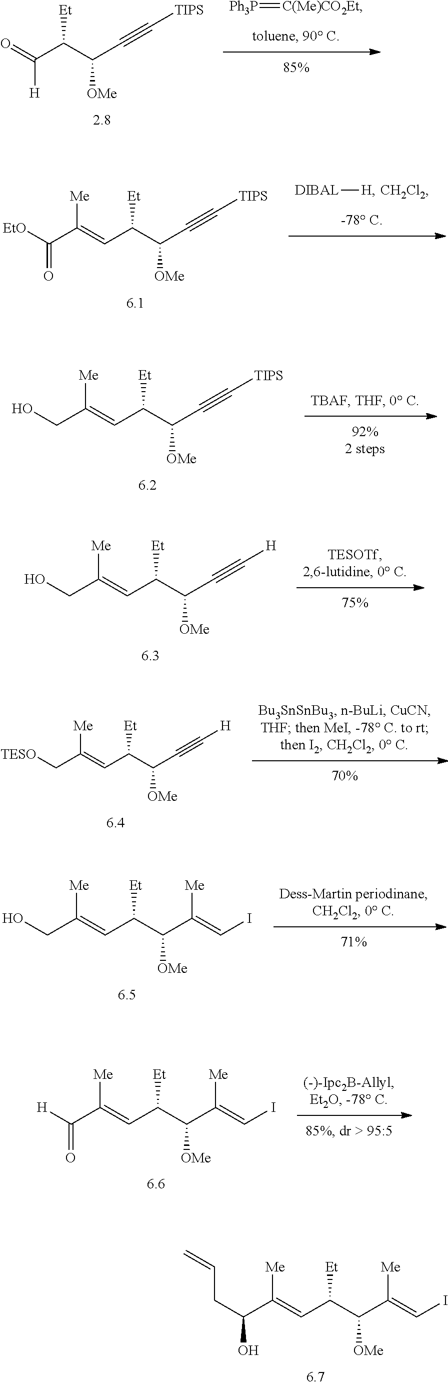

According to one embodiment, a method for the synthesis of a compound of Formula 6.7:

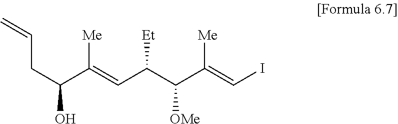 [SMILES: C=CC[C@H](O)/C(C)=C/[C@H](CC)[C@@H](OC)/C(C)=C/I]

comprises, in order:

(a) reacting a compound of Formula 2.8:

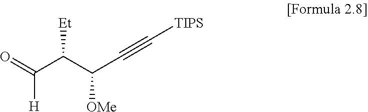

in the presence of Ph₃P═C(Me)CO₂Et and toluene at 90° C. to form a compound of Formula 6.1:

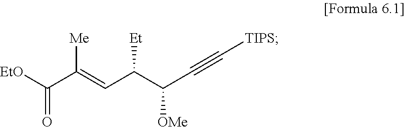

(b) reacting the compound of Formula 6.1 in the presence of diisobuutyl aluminum hydride (DIBAL) and dichloromethane (CH₂Cl₂) at −78° C. to form a compound of Formula 6.2:

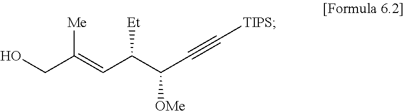

(c) reacting the compound of Formula 6.2 in the presence of tetra-n-butylammonium fluoride (TBAF), tetrahydrofuran (THF) at 0° C. to form a compound of Formula 6.3:

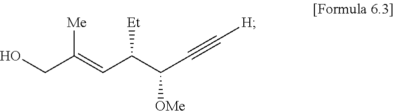

(d) reacting the compound of Formula 6.3 in the presence of in the presence of triethylsilyltrifluoromethane sulfonate (TESOTf), and 2,6-lutidine at 0° C. to form a compound of Formula 6.4:

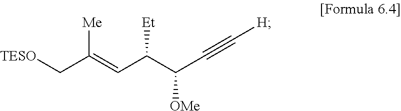

(e) reacting the compound of Formula 6.4 in the presence of tributyltin hydride (Bu₃SnSnBu₃), butyllithium (BuLi), copper (I) cyanide (CuCN), tetrahydrofuran (THF), then methyl iodide (MeI) at −78° C. to room temperature, then elemental iodine (I₂) and dichloromethane (CH2Cl2) at 0° C. to forma compound of Formula 6.5:

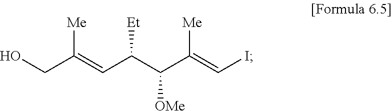 [SMILES: CC[C@@H](/C=C(\C)CO)[C@@H](OC)/C(C)=C/I]

(f) reacting the compound of Formula 6.5 in the presence of Dess-Martin periodinane and dichloromethane (CH2Cl2) at 0° C. to form a compound of Formula 6.6:

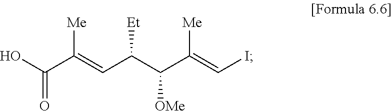 [SMILES: CC[C@@H](/C=C(\C)C(=O)O)[C@@H](OC)/C(C)=C/I]

and

(g) reacting the compound of Formula 6.6 in the presence of (−)-lpc₂B-Allyl diethylether at −78° C. to form the copound of Formula 6.7

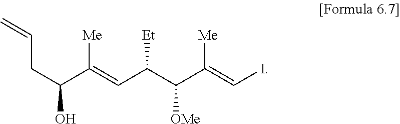 [SMILES: C=CC[C@H](O)/C(C)=C/[C@H](CC)[C@@H](OC)/C(C)=C/I]

Scheme 7.

The following synthetic scheme illustrates the generation of a fragment required to synthesize the correct structure of mangrolide Aglycon. According to one embodiment, a method for the synthesis of a compound of Formula 7.11:

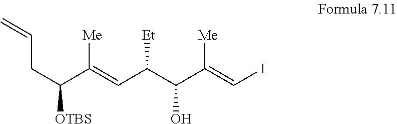 [SMILES: C=CC[C@H](O[Si](C)(C)C(C)(C)C)/C(C)=C/[C@H](CC)[C@@H](O)/C(C)=C/I]

comprises, in order:

(a) Reacting a compound of formula 2.1 in the presence of 3,4-dihydropyran, pyridinium p-toluene sulfonate (PPTS) and dichloromethane (CH₂Cl₂) to form a compound of Formula 2.2:

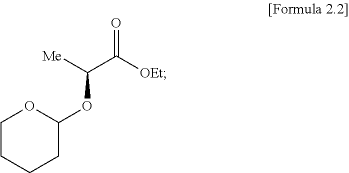 [SMILES: CCOC(=O)[C@H](C)OC1CCCCO1]

(b) Reacting the compound of Formula 2.2 in the presence of diisobutylaluminum hydride (DIBAL) amd dichloroethane (CH₂Cl₂) at −78° C. to form a compound of Formula 2.3:

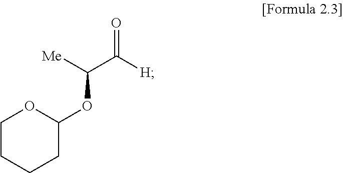

(c) Reacting the compound of Formula 2.3 in the presence of n-PrPh₃PBr, n-butyllilthium, tetrohydrofuran at −78° C. to room temperature to form a compound of formula 2.4:

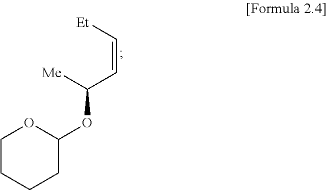 [SMILES: CC/C=C\[C@H](C)OC1CCCCO1]

(d) Reacting the compound of Formula 2.4 with tosylic acid (TsOH.H2)), ethylene glycol (HO(CH₂)2OH at 0° C. to form a compound of Formula 2.5:

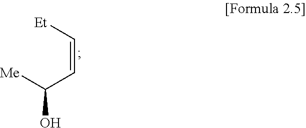

(e) Reacting the compound of Formula 2.5 with a compound of formula J

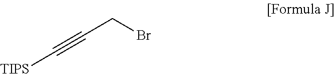

in the presence of sodium tert-butoxide (t-BuONa), tetrahydrofuran at 0° C. to form a compound of Formula 2.6;

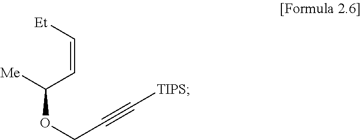

(f) Reacting the compound of Formula 2.6 in the presence of n-butyllithium, tetrahydrofuran (THF) at −78° C. and then methoxy methylether chloride (MOMCl) to form a compound of Formula 7.1:

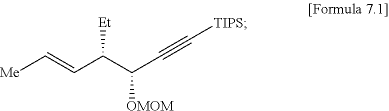

(g) Reacting the compound of Formula 7.1 in the presence of ozone (03), dichloromethane (CH₂Cl₂) at −78° C.; then dimethylsulfide (Me₂S) at room temperature to form a compound of Formula 7.2:

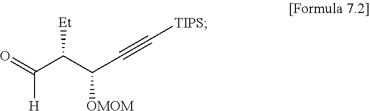

(h) Reacting the compound of Formula 7.2 in the presence of Ph₃P═C(Me)CO₂Et and toluene at 90° C. to form a compound of Formula 7.3:

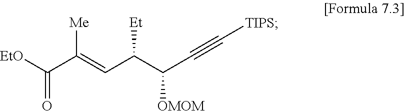

(i) Reacting the compound of Formula 7.3 in the presence of diisobuutylaluminum hydride (DIBAL) and dichloromethane (CH₂Cl₂) at −78° C. to form a compound of Formula 7.4:

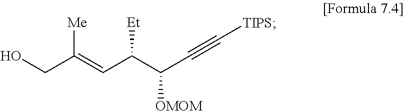

(j) Reacting the compound of Formula 7.4 in the presence of tetra-n-butylammonium fluorice (TBAF) and tetrahydrofuran (THF) at 0° C. to form a compound of Formula 7.5:

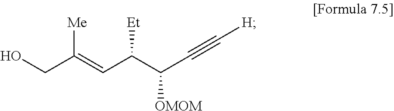

(k) Reacting the compound of Formula 7.5 in the presence of triethylsilyltrifluoromethane sulfonate (TESOTf), and 2,6-lutidine at 0° C. to form a compound of Formula 7.6:

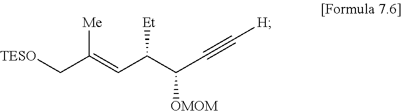

(l) reacting the compound of Formula 7.6 in the presence of tributyltin hydride (Bu₃SnSnBu₃), butyllithium (BuLi), copper (I) cyanide (CuCN), tetrahydrofuran (THF), then methyl iodide (MeI) at −78° C. to room temperature, then elemental iodine (I₂) and dichloromethane (CH₂Cl₂) at 0° C. to forma compound of Formula 7.7:

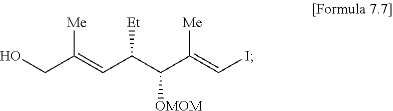

(m) reacting the compound of Formula 7.7 in the presence of Dess-Martin periodinane and dichloromethane (CH₂Cl₂) at 0° C. to form a compound of Formula 7.8:

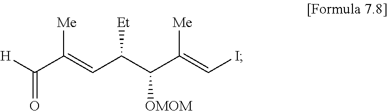

(n) reacting the compound of Formula 7.8 in the presence of (−)-lpc₂B-Allyl diethylether at −78° C. to form the compound of Formula 7.9

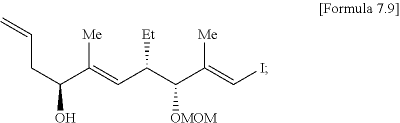

(o) reacting the compound of Formula 7.9 in the presence of triethylsilyltrifluoromethane sulfonate (TESOTf), and 2,6-lutidine at 0° C. to form a compound of Formula 7.10:

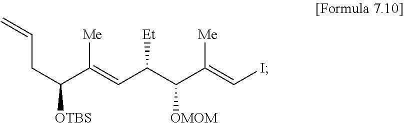

and

(p) reacting the compound of Formula 7.10 in the presence of boron trifluoride etherate (BF₃.OEt₂ and dimethyl sulfide to form a compound of Formula 7.11.

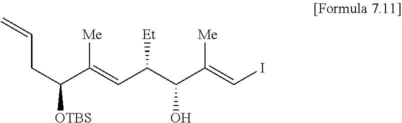 [SMILES: C=CC[C@H](O[Si](C)(C)C(C)(C)C)/C(C)=C/[C@H](CC)[C@@H](O)/C(C)=C/I]

Scheme 8.

The following synthetic scheme illustrates synthesis of the correct mangrolide Aglycon methylether structure C (8.3, top panel), and correct mangrolide Aglycon disilylether (8.5).

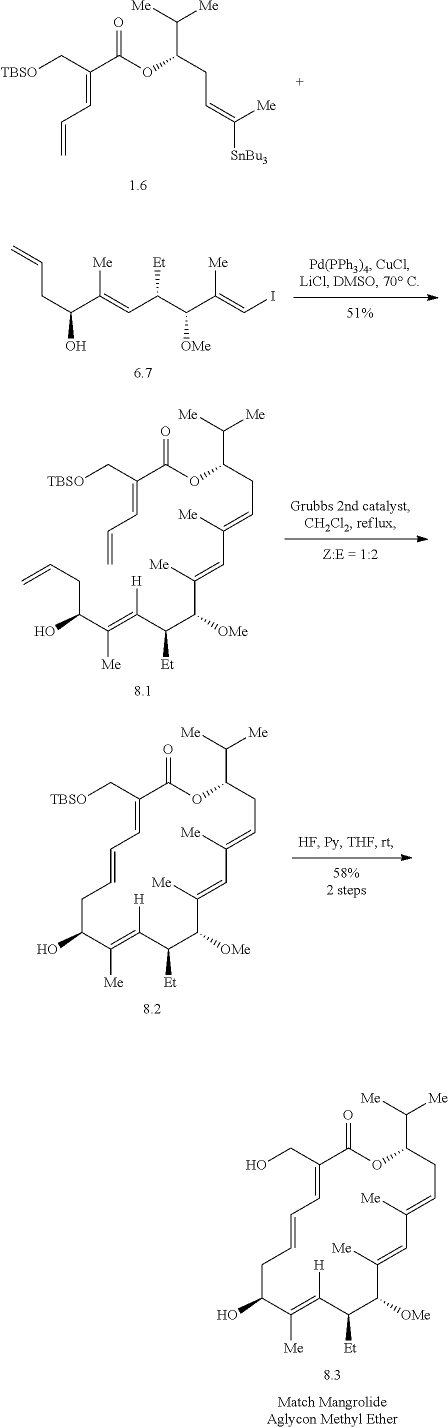

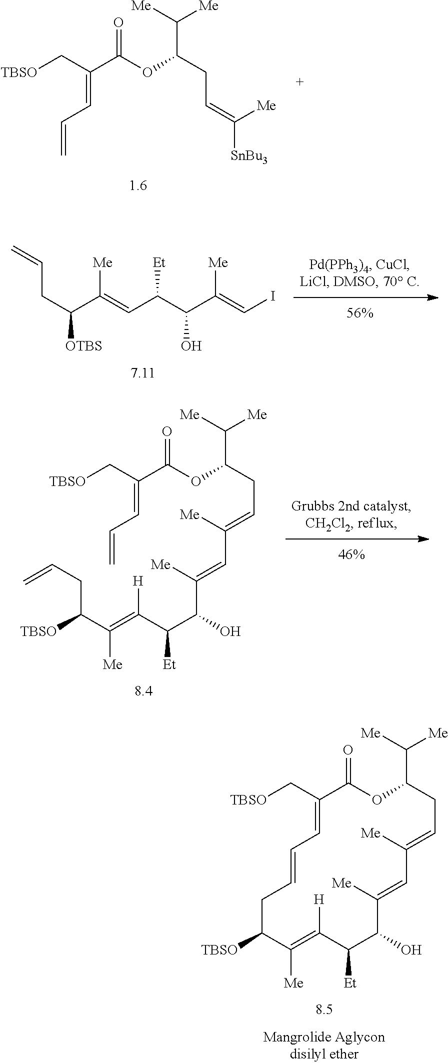

According to one embodiment, a method for synthesis of a compound of Formula 8.3:

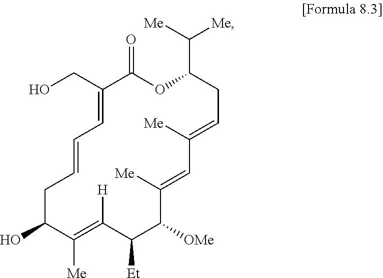 [SMILES: CC[C@H]1/C=C(\C)[C@@H](O)C/C=C/C=C(\CO)C(=O)O[C@H](C(C)C)C/C=C(C)/C=C(\C)[C@@H]1OC]

wherein the compound of Formula 8.3 matches mangrolide Aglycon methyl ether structure C

comprises, in order:

Reacting a compound of Formula 1.6:

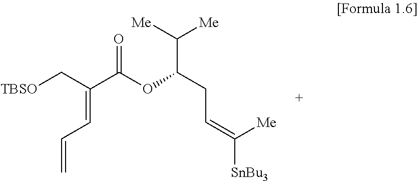

and a compound of Formula 6.7:

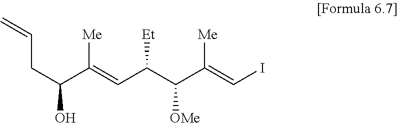 [SMILES: C=CC[C@H](O)/C(C)=C/[C@H](CC)[C@@H](OC)/C(C)=C/I]

in the presence of the catalyst tetrakis(triphenylphosphine)palladium (Pd(PPh₃)₄), CuCl, LiCl, and dimethylsulfoxide (DMSO) at 70° C. to form a compound of Formula 8.1:

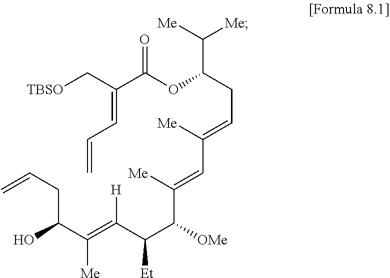 [SMILES: C=C/C=C(\CO[Si](C)(C)C(C)(C)C)C(=O)O[C@@H](C/C=C(C)/C=C(\C)[C@H](OC)[C@H](/C=C(\C)[C@@H](O)CC=C)CC)C(C)C]

(b) reacting the compound of Formula 8.1 in the presence of (i) Grubbs 2^{nd} catalyst, and dichloromethane (CH2Cl2) with reflux to form a compound of Formula 8.2:

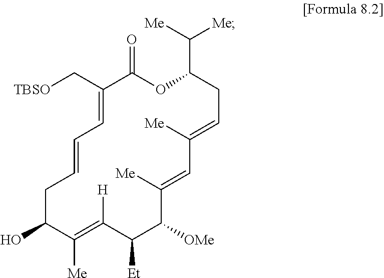 [SMILES: CC[C@H]1/C=C(\C)[C@@H](O)C/C=C/C=C(\CO[Si](C)(C)C(C)(C)C)C(=O)O[C@H](C(C)C)C/C=C(C)/C=C(\C)[C@@H]1OC]

and

(c) reacting the compound of Formula 8.2 in the presence of hydrofluoric acid (HF), pyridine (Py) and tetrahydrofuran (THF) at room temperature to form the compound of Formula 8.3.

According to another embodiment, a method for the synthesis of a compound of Formula 8.5:

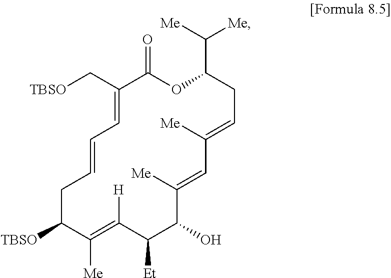 [SMILES: CC[C@H]1/C=C(\C)[C@@H](O[Si](C)(C)C(C)(C)C)C/C=C/C=C(\CO[Si](C)(C)C(C)(C)C)C(=O)O[C@H](C(C)C)C/C=C(C)/C=C(\C)[C@@H]1O]

wherein the compound of Formula 8.5 is synthetic mangrolide Aglycon disilyl ether of correct structure,

comprises, in order:

(a) reacting a compound of Formula 1.6

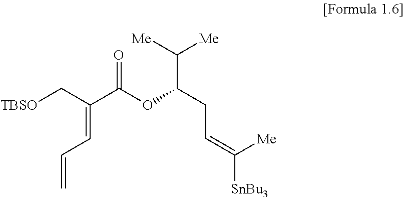

and a compound of Formula 7.11

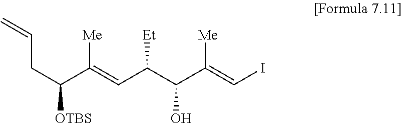 [SMILES: C=CC[C@H](O[Si](C)(C)C(C)(C)C)/C(C)=C/[C@H](CC)[C@@H](O)/C(C)=C/I]

in the presence of the catalyst tetrakis(triphenylphosphine)palladium (Pd(PPh₃)₄), CuCl, LiCl, and dimethylsulfoxide (DMSO) at 70° C. to form a compound of Formula 8.4:

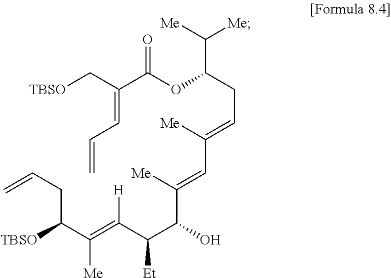 [SMILES: C=C/C=C(\CO[Si](C)(C)C(C)(C)C)C(=O)O[C@@H](C/C=C(C)/C=C(\C)[C@H](O)[C@H](/C=C(\C)[C@H](CC=C)O[Si](C)(C)C(C)(C)C)CC)C(C)C]

and

(b) reacting the compound of formula 8.4 in the presence of Grubbs 2^{nd} catalyst and dichloromethane (CH₂Cl₂) under reflux conditions to form the compound of Formula 8.5:

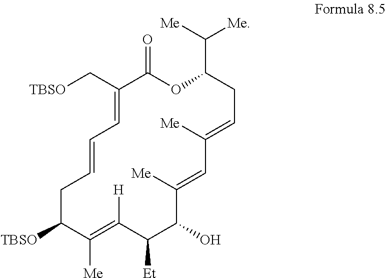 [SMILES: CC[C@H]1/C=C(\C)[C@@H](O[Si](C)(C)C(C)(C)C)C/C=C/C=C(\CO[Si](C)(C)C(C)(C)C)C(=O)O[C@H](C(C)C)C/C=C(C)/C=C(\C)[C@@H]1O]

Scheme 10.

The following synthetic scheme illustrates synthesis of a mangrolide A deoxyglucose fragment of Formula 10.9:

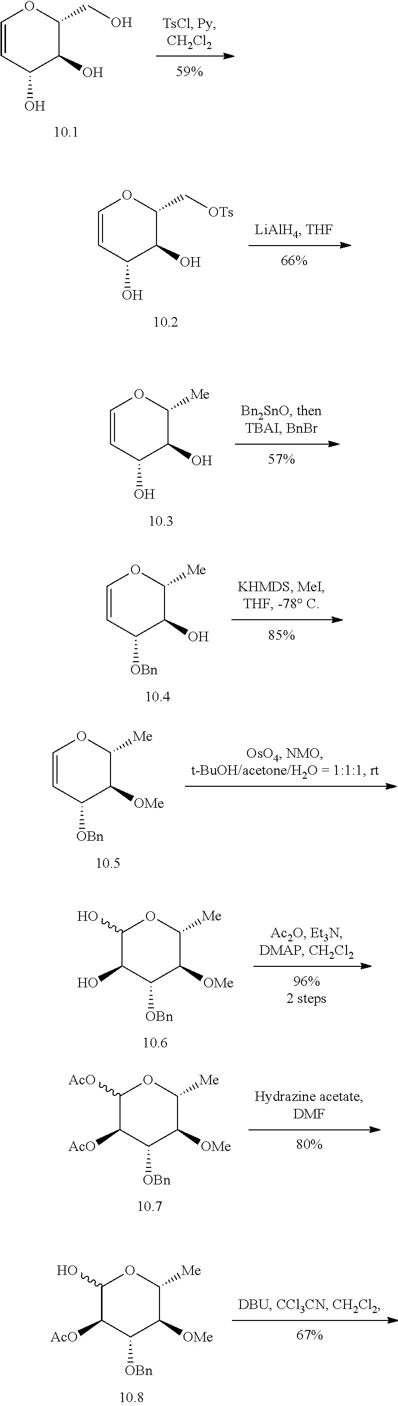

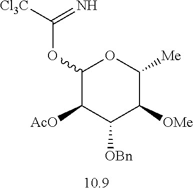

According to an embodiment, a method for the synthesis of a mangrolide A deoxyglucose fragment of Formula 10.9

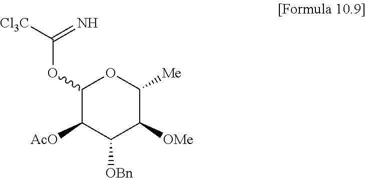

comprises, in order:

(a) Reacting a compound of Formula 10.1 in the presence of p-toluenesulfonyl chloride (TsCl), Pyridine (Py) and dichloromethane (CH₂Cl₂) to form a compound of formula 10.2:

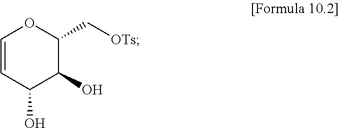 [SMILES: Cc1ccc(S(=O)(=O)OC[C@H]2OC=C[C@@H](O)[C@@H]2O)cc1]

(b) Reacting the compound of Formula 10.2 in the presence of lithium aluminum hydride (LiAlH₄) and tetrahydrofuran (THF) to form a compound of Formula 10.3:

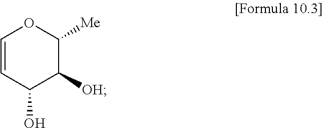

(c) reacting the compound of Formula 10.3 in the presence of dibenzyl tin (II) oxide (Bn₂SnO), and then tetrabutylammonium iodide (TBAI) and benzyl bromide (BnBr) to form a compound of Formula 10.4:

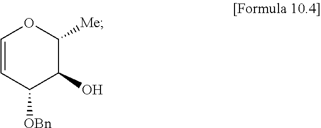 [SMILES: C[C@H]1OC=C[C@@H](OCc2ccccc2)[C@@H]1O]

(d) reacting the compound of formula 10.4 in the presence of potassium bis(trimethylsilyl)amide (KHMDS), methyl iodide (MeI) and tetrahydrofuran (THF) at −78° C. to form a compound of Formula 10.5:

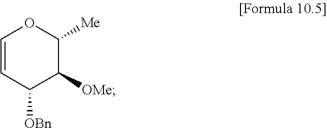 [SMILES: CO[C@@H]1[C@@H](C)OC=C[C@H]1OCc1ccccc1]

(e) reacting the compound of Formula 10.5 in the presence of osmium tetroxide (OsO₄), N-methylmorpholine-N-oxide (NMO), and tert-butanol/acetone/water (1:1:1) at room temperature to form a compound of Formula 10.6;

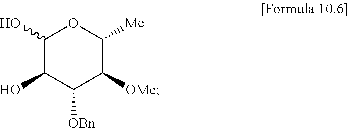 [SMILES: CO[C@H]1[C@H](OCc2ccccc2)[C@@H](O)C(O)O[C@@H]1C]

(f) reacting the compound of Formula 10.6 in the presence of acetic anhydride (Ac₂O), triethylamine (Et3N), 4-(dimethylamino)pyridine (DMAP), and dichloromethane (CH₂Cl₂) to form a compound of Formula 10.7:

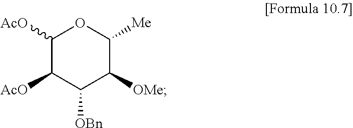 [SMILES: CO[C@H]1[C@H](OCc2ccccc2)[C@@H](OC(C)=O)C(OC(C)=O)O[C@@H]1C]

(g) reacting the compound of Formula 10.7 in the presence of hydrazine acetate and dimethylformamide (DMF) to form a compound of Formula 10.8:

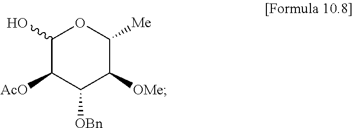 [SMILES: CO[C@H]1[C@H](OCc2ccccc2)[C@@H](OC(C)=O)C(O)O[C@@H]1C]

and

(h) reacting the compound of Formula 10.8 in the presence of 1,8-diazabicycloundec-7-ene (DBU), trichloroacetonitrile (CCl₃CN) and dichloromethane (CH₂Cl₂) to form the compound of Formula 10.9:

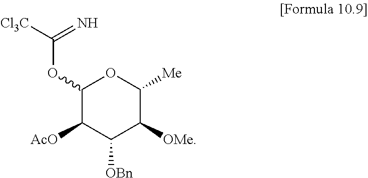

Scheme 11.

The following synthetic scheme illustrates synthesis of mangrolide A mycaminose sugar via degradation of kitasamycin.

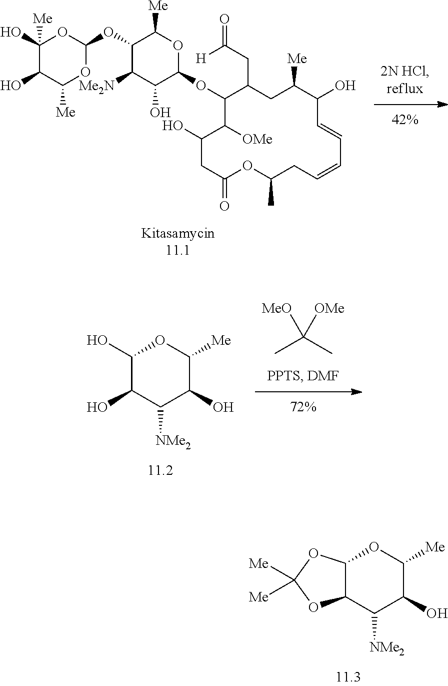

According to one embodiment, a method for the synthesis of a mangrolide A mycaminose sugar of Formula 11.3

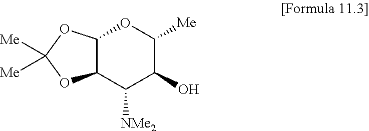 [SMILES: C[C@H]1O[C@H]2OC(C)(C)O[C@@H]2[C@@H](N(C)C)[C@@H]1O]

comprises, in order:

(a) reacting a compound of Formula 11.1:

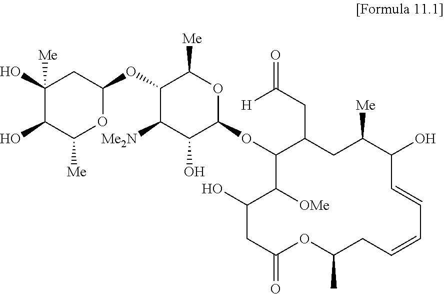 [SMILES: COC1C(O)CC(=O)O[C@H](C)C/C=C\C=C\C(O)[C@H](C)CC(CC=O)C1O[C@@H]1O[C@H](C)[C@@H](O[C@@H]2C[C@](C)(O)[C@H](O)[C@@H](C)O2)[C@H](N(C)C)[C@H]1O]

in the presence of 2N HCl under reflux conditions, to form a compound of Formula 11.2:

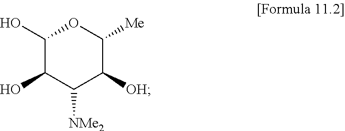

and

(b) reacting the compound of formula 11.2 in the presence of

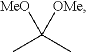

pyridinium toluene-4-sulfonate (PPTS) and dimethylformamide (DMF) to form the compound of Formula 11.3.

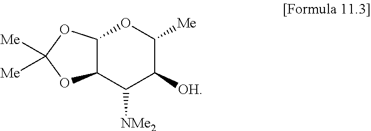

Scheme 12.

The following synthetic scheme illustrates the synthesis of a disaccharide fragment of mangrolide A.

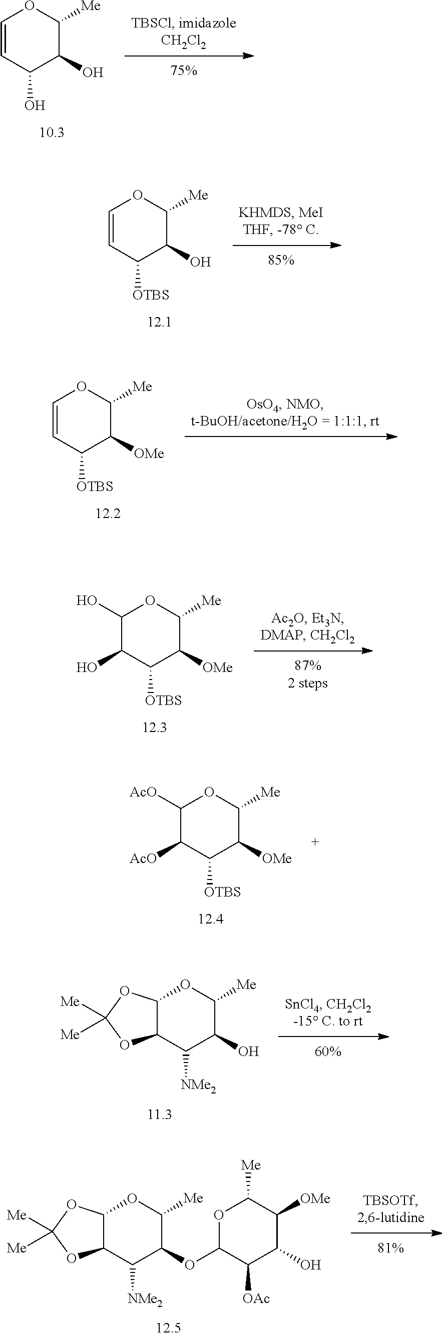

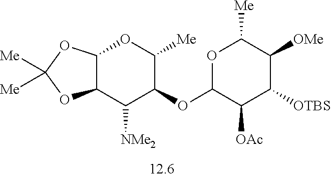

According to one embodiment, a method for the synthesis of a disachharide fragment of mangrolide A of Formula 12.6

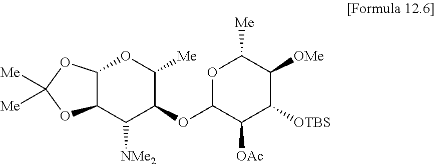

comprises, in order:

(a) reacting a compound of Formula 10.3:

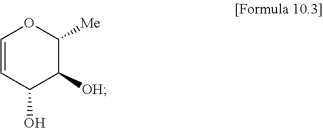

in the presence of tert-butyldimethylsilyl chloride (TBSCl), imidazone, and dichloromethane (CH₂Cl₂) to form a compound of Formula 12.1:

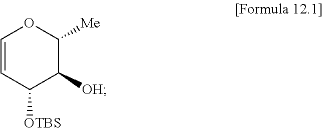 [SMILES: C[C@H]1OC=C[C@@H](O[Si](C)(C)C(C)(C)C)[C@@H]1O]

(b) reacting the compound of Formula 12.1 in the presence of potassium bis(trimethylsilyl)amide (KHMDS), methyl iodide (MeI) and tetrahydrofuran (THF) at −78° C. to form a compound of Formula 12.2:

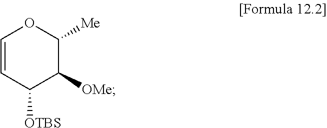 [SMILES: CO[C@@H]1[C@@H](C)OC=C[C@H]1O[Si](C)(C)C(C)(C)C]

(c) reacting the compound of Formula 12.2 in the presence of osmium tetroxide (OsO₄), N-methylmorpholine-N-oxide (NMO), and tert-butanol/acetone/water (1:1:1) at room temperature to form a compound of Formula 12.3:

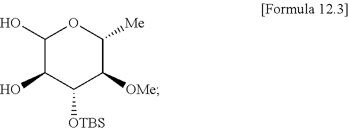 [SMILES: CO[C@H]1[C@H](O[Si](C)(C)C(C)(C)C)[C@@H](O)C(O)O[C@@H]1C]

(d) reacting the compound of Formula 12.3 in the presence of acetic anhydride (Ac2O), triethylamine (Et3N), 4-(dimethylamino)pyridine (DMAP), and dichloromethane (CH2Cl2) to form a compound of Formula 12.4:

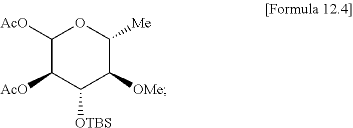 [SMILES: CO[C@H]1[C@H](O[Si](C)(C)C(C)(C)C)[C@@H](OC(C)=O)C(OC(C)=O)O[C@@H]1C]

(e) reacting the compound of Formula 12.4 with a compound of Formula 11.3:

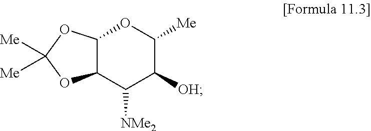

in the presence of tin (IV) chloride (SnCl4) at −15° C. to room temperature to form a compound of Formula 12.5:

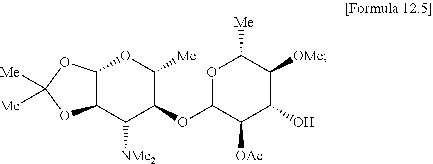

and

(f) reacting the compound of Formula 12.5 in the presence of trifluoromethylsilyl trifluoromethane sulfonate (TBSOTf) and 2,6-lutidine to form the compound of Formula 12.6.

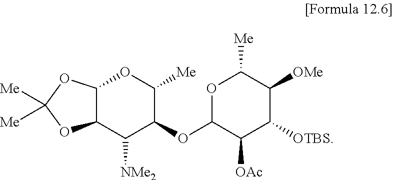

Scheme 13

shows an alternative synthetic scheme for synthesis of a mangrolide disaccharide fragment.

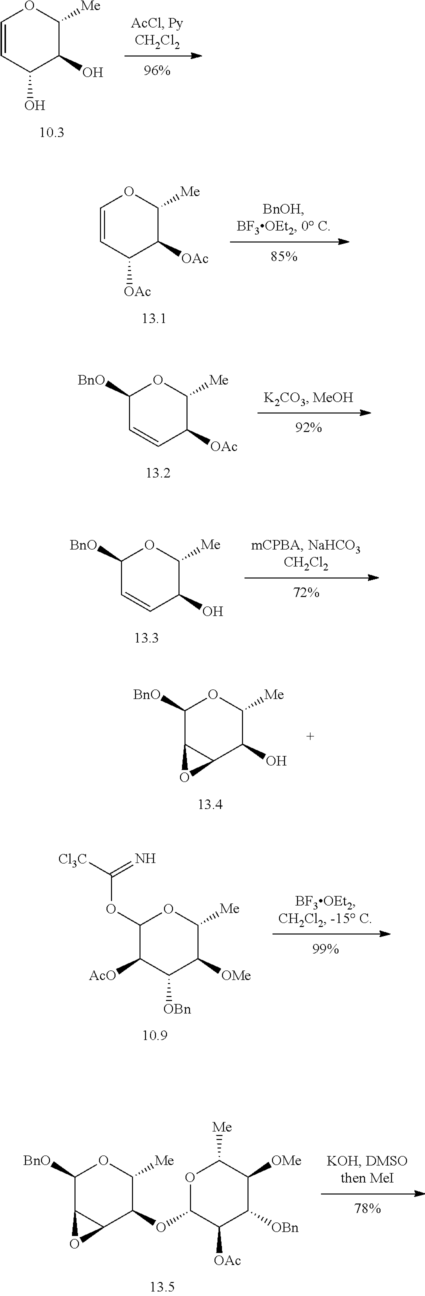

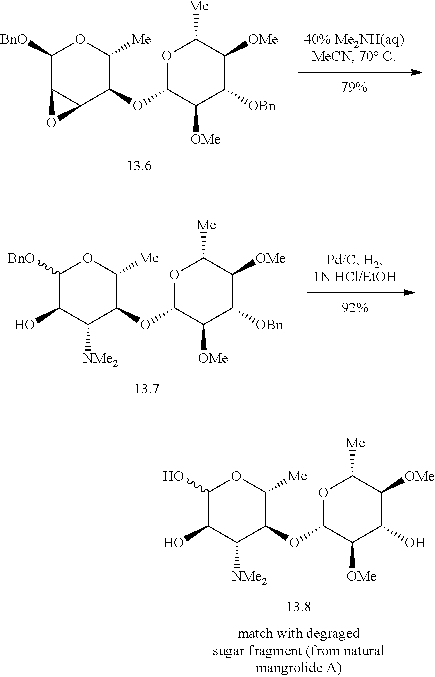 [SMILES: CO[C@H]1[C@H](O[C@H]2[C@H]3O[C@H]3[C@@H](OCc3ccccc3)O[C@@H]2C)O[C@H](C)[C@@H](OC)[C@@H]1OCc1ccccc1 ; CO[C@H]1[C@H](O[C@H]2[C@H](N(C)C)[C@@H](O)C(OCc3ccccc3)O[C@@H]2C)O[C@H](C)[C@@H](OC)[C@@H]1OCc1ccccc1 ; CO[C@H]1[C@H](O[C@H]2[C@H](N(C)C)[C@@H](O)C(O)O[C@@H]2C)O[C@H](C)[C@@H](OC)[C@@H]1O]

According to this embodiment, a method for the synthesis of a compound of Formula 13.8

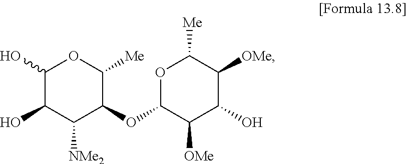 [SMILES: CO[C@H]1[C@H](O[C@H]2[C@H](N(C)C)[C@@H](O)C(O)O[C@@H]2C)O[C@H](C)[C@@H](OC)[C@@H]1O]

wherein the compound of Formula 13.8 matches the degraded sugar fragment from natural mangrolide A,

comprises, in order

(a) reacting a compound of Formula 10.3:

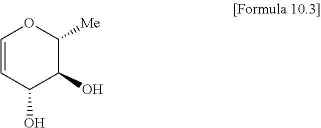

in the presence of acetylchloride (AcCl), pyridine (Py) and dichloromethane (CH₂Cl₂) to form a compound of Formula 13.1:

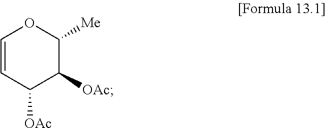 [SMILES: CC(=O)O[C@@H]1[C@@H](C)OC=C[C@H]1OC(C)=O]

(b) reacting the compound of Formula 13.1 in the presence of benzyl alcohol (BnOH), boron trifluoride etherate (BF3.OEt2) at 0° C. to form a compound of Formula 13.2:

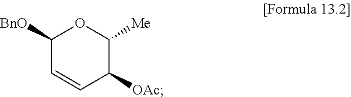 [SMILES: CC(=O)O[C@H]1C=C[C@@H](OCc2ccccc2)O[C@@H]1C]

(c) reacting the compound of Formula 13.2 in the presence of posassium carbonate (K2CO3) and methanol (MeOH) to form a compound of Formula 13.3:

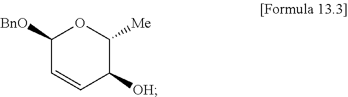 [SMILES: C[C@H]1O[C@H](OCc2ccccc2)C=C[C@@H]1O]

(d) reacting the compound of Formula 13.3 in the presence of meta-clhoroperoxybenzoic acid (mCPBA), sodium bicarbonate (NaHCO3), and dichlromethane (CH₂Cl₂) to form a compound of Formula 13.4;

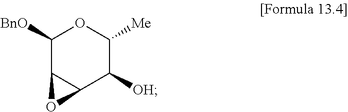 [SMILES: C[C@H]1O[C@H](OCc2ccccc2)[C@@H]2O[C@@H]2[C@@H]1O]

(e) reacting the compound of Formula 13.4 with a compound of formula 10.9:

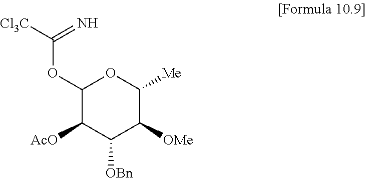

in the presence of boron trifluoride etherate, and dichloromethane at −15 C to form a compound of Formula 13.5:

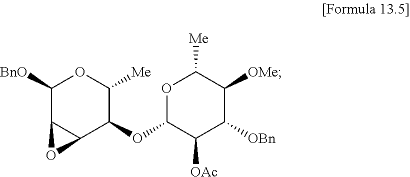 [SMILES: CO[C@H]1[C@H](OCc2ccccc2)[C@@H](OC(C)=O)[C@H](O[C@H]2[C@H]3O[C@H]3[C@@H](OCc3ccccc3)O[C@@H]2C)O[C@@H]1C]

(f) reacting the compound of formula 13.5 in the presence of potassium hydroxide (KOH), DMSO then methyl iodide (MeI) to form a compound of Formula 13.6:

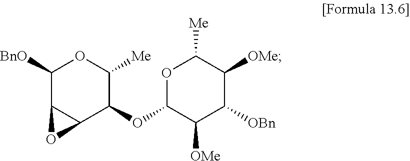 [SMILES: CO[C@H]1[C@H](O[C@H]2[C@H]3O[C@H]3[C@@H](OCc3ccccc3)O[C@@H]2C)O[C@H](C)[C@@H](OC)[C@@H]1OCc1ccccc1]

(g) reacting the compound of formula 13.6 in the presence of dimethylamine (Me2NH (aq)) and acetonitrile (MeCN) at 70° C. to form the compound of Formula 13.7:

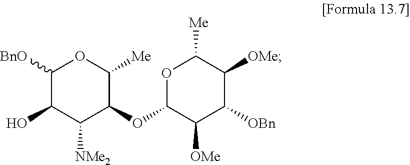 [SMILES: CO[C@H]1[C@H](O[C@H]2[C@H](N(C)C)[C@@H](O)C(OCc3ccccc3)O[C@@H]2C)O[C@H](C)[C@@H](OC)[C@@H]1OCc1ccccc1]

and

(h) reacting the compound of Formula 13.7 in the presence of palladium on carbon (Pd/C), hydrogen gas (H₂), 1N HCl and ethanol (EtOH) to form the compound of Formula 13.8:

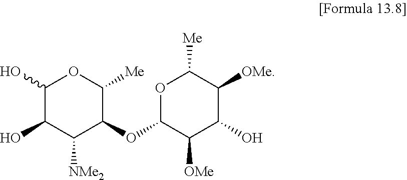 [SMILES: CO[C@H]1[C@H](O[C@H]2[C@H](N(C)C)[C@@H](O)C(O)O[C@@H]2C)O[C@H](C)[C@@H](OC)[C@@H]1O]

Scheme 14

is a synthetic scheme illustrating synthesis of fully synthetic, biologically active Mangrolide A.

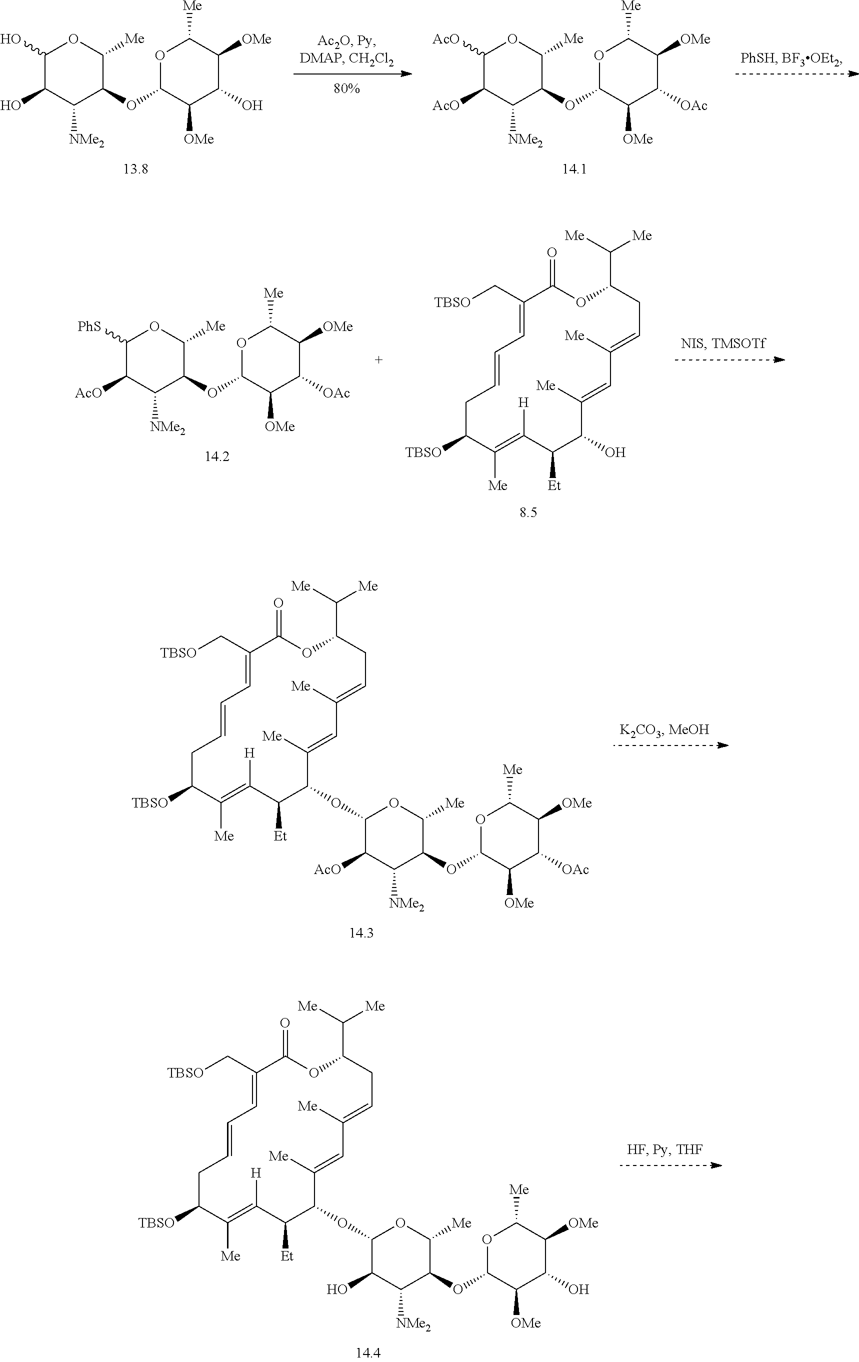

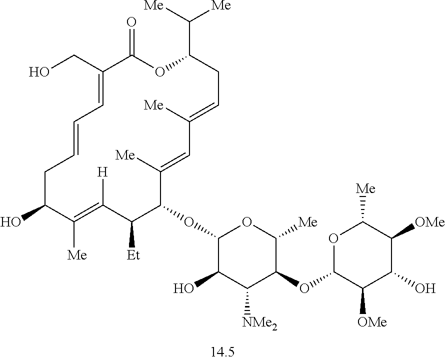 [SMILES: CC[C@H]1/C=C(\C)[C@@H](O)C/C=C/C=C(\CO)C(=O)O[C@H](C(C)C)C/C=C(C)/C=C(\C)[C@@H]1O[C@@H]1O[C@H](C)[C@@H](O[C@@H]2O[C@H](C)[C@@H](OC)[C@H](O)[C@H]2OC)[C@H](N(C)C)[C@H]1O]

According to one embodiment, a method for the synthesis of a fully synthetic, biologically active mangrolide A of Formula 14.5:

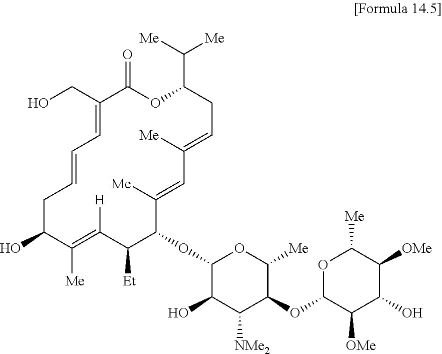 [SMILES: CC[C@H]1/C=C(\C)[C@@H](O)C/C=C/C=C(\CO)C(=O)O[C@H](C(C)C)C/C=C(C)/C=C(\C)[C@@H]1O[C@@H]1O[C@H](C)[C@@H](O[C@@H]2O[C@H](C)[C@@H](OC)[C@H](O)[C@H]2OC)[C@H](N(C)C)[C@H]1O]

comprises, in order

(a) reacting a compound of Formula 13.8:

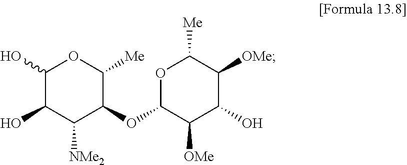 [SMILES: CO[C@H]1[C@H](O[C@H]2[C@H](N(C)C)[C@@H](O)C(O)O[C@@H]2C)O[C@H](C)[C@@H](OC)[C@@H]1O]

in the presence of acetyic anhydride (Ac₂O), pyridine (Py), 4-dimethylaminopyridine (DMAP), and dichloromethane (CH₂Cl₂) to form a compound of Formula 14.1:

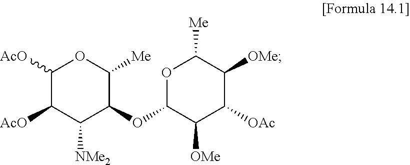 [SMILES: CO[C@H]1[C@H](O[C@H]2[C@H](N(C)C)[C@@H](OC(C)=O)C(OC(C)=O)O[C@@H]2C)O[C@H](C)[C@@H](OC)[C@@H]1OC(C)=O]

(b) reacting the compound of Formula 14.1 in the presence of thiophenol (PhSH) and Boron trifluoride diethyl etherate (BF₃OEt₂) to form a compound of Formula 14.2;

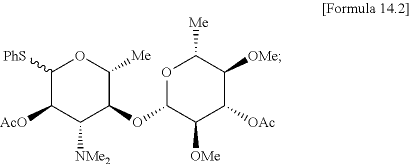

(c) reacting the compound of Formula 14.2 and a compound of Formula 8.5:

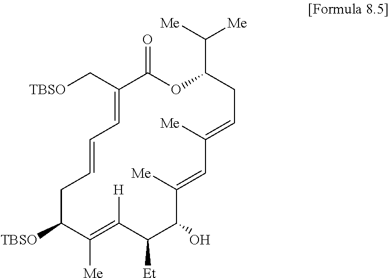 [SMILES: CC[C@H]1/C=C(\C)[C@@H](O[Si](C)(C)C(C)(C)C)C/C=C/C=C(\CO[Si](C)(C)C(C)(C)C)C(=O)O[C@H](C(C)C)C/C=C(C)/C=C(\C)[C@@H]1O]

in the presence of n-iodosuccinimide (NIS) and trimethylsilyl trifluoromethanesulfonate (TMSOf) to form a compound of Formula 14.3:

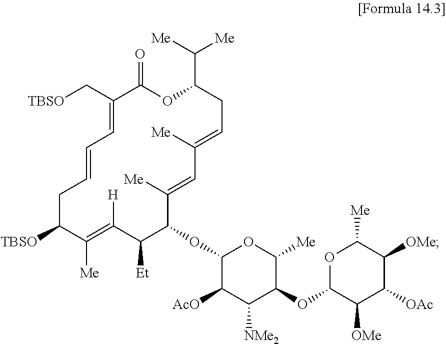 [SMILES: CC[C@H]1/C=C(\C)[C@@H](O[Si](C)(C)C(C)(C)C)C/C=C/C=C(\CO[Si](C)(C)C(C)(C)C)C(=O)O[C@H](C(C)C)C/C=C(C)/C=C(\C)[C@@H]1O[C@@H]1O[C@H](C)[C@@H](O[C@@H]2O[C@H](C)[C@@H](OC)[C@H](OC(C)=O)[C@H]2OC)[C@H](N(C)C)[C@H]1OC(C)=O]

(d) reacting the compound of Formula 14.3 in the presence of potassium carbonate (K₂CO₃) and methanol (MeOH) to form a compound of Formula 14.4:

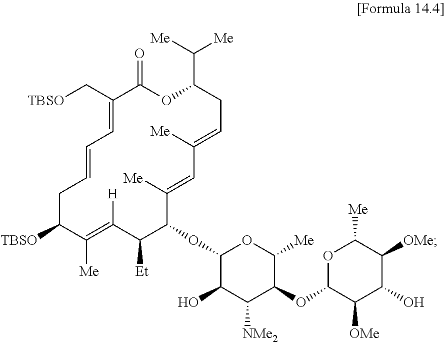 [SMILES: CC[C@H]1/C=C(\C)[C@@H](O[Si](C)(C)C(C)(C)C)C/C=C/C=C(\CO[Si](C)(C)C(C)(C)C)C(=O)O[C@H](C(C)C)C/C=C(C)/C=C(\C)[C@@H]1O[C@@H]1O[C@H](C)[C@@H](O[C@@H]2O[C@H](C)[C@@H](OC)[C@H](O)[C@H]2OC)[C@H](N(C)C)[C@H]1O]

and

(e) reacting the compound of Formula 14.4 in the presence of hydrofluoric acid (HF), pyridine (Py) and tetrahydrofuran (THF) to form the compound of Formula 14.5:

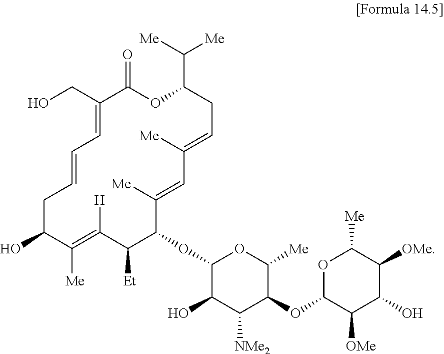 [SMILES: CC[C@H]1/C=C(\C)[C@@H](O)C/C=C/C=C(\CO)C(=O)O[C@H](C(C)C)C/C=C(C)/C=C(\C)[C@@H]1O[C@@H]1O[C@H](C)[C@@H](O[C@@H]2O[C@H](C)[C@@H](OC)[C@H](O)[C@H]2OC)[C@H](N(C)C)[C@H]1O]

### Example 2

### Comparison of Natural Mangrolide A to Synthetic Isomers of Mangrolide A by NMR

The distance between peaks in a multiplet is called the coupling constant, J. The coupling constant J is calculated by taking the distance (in ppm) between any two adjacent split peaks.

For Natural Mangrolide M_{H9-H10}=9.7 Hz and J_{H10-H11}=10.5 Hz.

For Synthetic Mangrolide A, H_{9-H10=9.6} Hz and J_{H10-H11}=10.6 Hz.

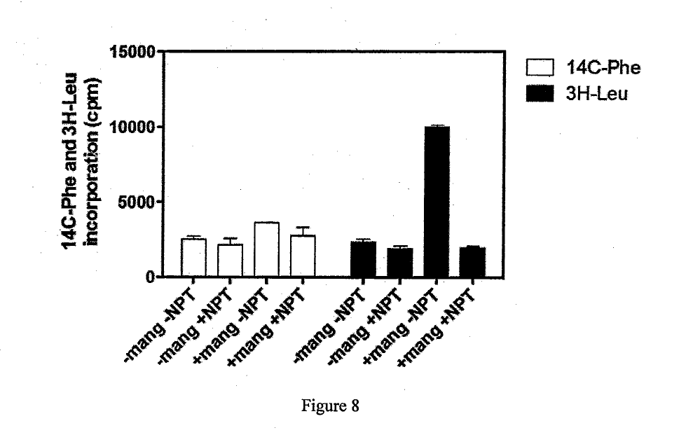

For an isomeric synthetic Mangrolide A, J_{H9-H10}=9.5 Hz and J_{H10-H11}=10.0 Hz. (FIG. 8)

Hydrogen spectra of natural Mangrolide A and synthetic Mangrolide A (same isomer) are shown in FIG. 9.

### Example 3

### Antibiotic Activity of Mangrolide A

Minimal Inhibitory Concentration (MIC) Test

The MIC test was performed by adding 2.0 mL of an antibiotic solution (100 ug/mL) to the first tube (out of, e.g., 9 tubes). 1.0 mL of sterile broth was added to all other tubes. 1.0 mL was transferred from the first tube to the second tube. Using a separate pipette, the contents of this tube were mixed and 1.0 mL was transferred to the third tube. Dilutions were continued in this manner through tube number 8. 1.0 mL was removed from tube 8 and discarded. The ninth tube, which serves as a control, did not contain an antibiotic. Colonies of the culture were suspended to an appropriate turbidity to be tested in 5.0 mL of Mueller-Hinton broth to give a slightly turbid suspension. This suspension was diluted by aseptically pipetting 0.2 mL of the suspension into 40 mL of Mueller-Hinton broth. 1.0 mL of the diluted culture suspension was added to each of the tubes. The final concentration of the antibiotic was one-half of the original concentration in each tube. All tubes were incubated at 35° C. overnight. The tubes were examined for visible signs of bacterial growth. The highest dilution without growth was the minimal inhibitory concentration (MIC).

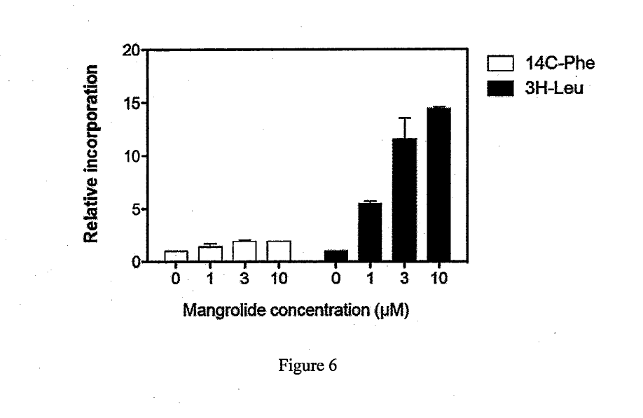

Antibiotic activity, measured as MIC in μg/mL, of natural Mangrolide A and a synthetic Mangrolide A O-methyl ether derivative were compared against the following Gram-negative microbial pathogens: Burkholderia cenocepacia J2315, Burkholderia cenocepacia, Pseudomonas aeruginosa PAO1, Acinetobacter Baumannii BAA-1793, Acinetobacter baumannii BAA-1605, Escherichia coli (EHEC), and Klebsiella pneumoniae. Natural Mangrolide A was a more potent antibiotic against all Gram-negative bacteria tested when compared with the Mangrolide A O-methyl ether derivative. Mangrolide A was not as effective against the Gram positive bacteria tested, Staphyloccus aureus (MIC_MIC 32) and Bacillus subtillis (MIC>32) as as it was against the Gram-negative bacterial pathogens tested (MIC range, 0.25-1). (FIG. 6) This experiment shows that the disachharide moiety of Mangrolide A is important for its antibacterial activity.

### Example 4

### Mangrolide Induces Misincorporation of Amino Acids

Error Frequency In Vitro Protein Synthesis Assay Using Natural mRNA

72 mM Tris-hydrochloride pH 7.5, 72 mM NH₄Cl, 12 mM magnesium acetate, 2.4 mM dithiothreitol (Sigma Co.), 2 mM adenosine triphosphate (ATP), 0.1 mM guanosine triphosphate (GTP), 5 mM phosphoenolpyruvate (Sigma Co.), 20 ug of pyruvate kinase (Sigma Co.) per mL, a mixture of 19 amino acids (each at 72 uM), 13 uM [¹⁴C]leucine (50 counts/min per picomole), 50 ug of calcium folinate (S.P.E.C.I.A., Paris, France) per mL, 0.2 mM DFP, and 0.6 mM Mg Titriplex in a final volume of 0.125 mL per tube. mRNA (1300 μg), an S-30 extract from E. coli (200 μg), or ribosomes plus S-150 (200 μg) and crude initiation factors were added. Incubations were for 30 minutes at 37° C. The extent of amino acid incorporation was determined by measuring the radioactivity remaining insoluble after treatment with hot trichloroacetic acid (90° C. for 15 min). To reduce endogenous mRNA, ribosomes plus 5-150 or S-30 extracts were preincubated just prior to use at 37° C. for 15 min in the reaction mixture described above without [¹⁴C]leucine, the energy generating system and added mRNA.

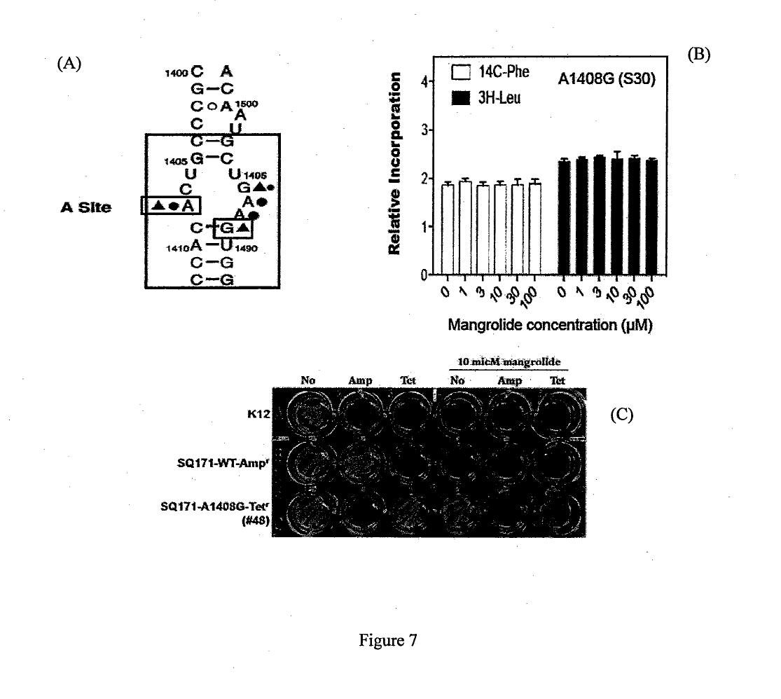

30S ribosomal extract, when in the presence of increasing concentrations (μM) of Mangrolide A, resulted in increased misincorporation of amino acids during protein synthesis. (FIG. 7)

Assay for Polyuridylic Acid [Poly(U)]-Directed-Polyphenylalanine Synthesis.

The reaction mixture contained 400 ug of E. coli B transfer RNA (General Biochemicals, Chagrin Falls, Ohio) per mL, 5 mM spermidine, and [¹⁴C]leucine was replaced with 13 μM [¹⁴C]phenylalanine (25° C. unts/min/pmol). A poly(UUU) RNA template [5′-GCGGCAAGGAGGUAAAUAAUG(UUU)₁₂UAAGCAGG-3′ (SEQ ID NO: 1) was used at a concentration of 100 ug per assay. (Legault-Demare and Chambliss, Journal of Bacteriology, 1974, 120(3): 1300-1307).

S30 rRNA Ribosomal Mutant is Resistant to Mangrolide A

The error frequency assay was conducted to determine whether a 30S rRNA ribosomal mutant, A1408G is susceptible to Mangrolide A. The mutation site is shown in FIG. 8A. When increasing concentrations of Mangrolide A were added to the assay, limited misincorporation was induced. (FIG. 8B) When wild type or A1408G (S30) mutant bacteria were grown in wells of a microwell plate containing no additions (No—negative control), Ampicillin or Tetracyclin in the presence or absence of Mangrolide A, A1408G (S30) mutants proliferated similarly to the proliferation of wild type bacteria grown in the absence of Mangrolide A. (FIG. 8C). Thus, the A1408G mutant is resistant to Mangrolide A.

Mangrolide A is Inactivated by Neomycin Phosphotransferase-1

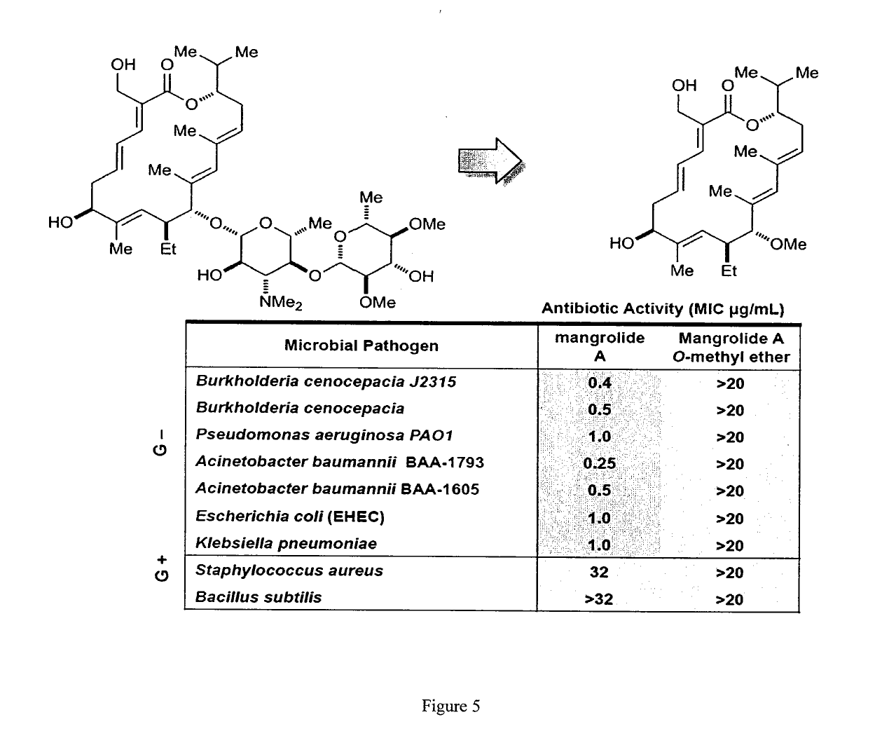

The assay for polyuridylic acid [poly(U)]-directed-polyphenylalanine synthesis was conducted in the presence or absence of neomycin phosphotransferase-1 (NPT). Wild type E. coli and E. coli transformed with a resistance plasmid expressing neomycin phosphotransferase-1 (NPT) were treated with Mangrolide A and translational error frequency determined. (FIG. 5) E. coli expressing NPT grown in the presence of Mangrolide A were resistant to the antibiotic activity of Mangrolide A, and showed error frequency comparable to wild type E. coli and E. coli transformed with a resistance plasmid expressing neomycin phosphotransferase-1 (NPT) without exposure to Mangrolide A. Therefore, the antibiotic activity of Mangrolide A against E. coli was inactivated by the expressed neomycin phosphotransferase-1 activity in the transformed strain.

Based on these results it was concluded that Mangrolide A binds to the 30S ribosomal subunit, similar to the mechanism of the aminoglycoside class of antibiotics. In contrast, telithromycin (a ketolide antibiotic, which differs chemically from a macrolide antibiotic by lack of α-L-cladinose at position 3 of the erythromolide A ring and has C11-C12 carbamate substituted by an imidazolyl and pyridyl ring through a buytyl chain) and erythromycin, a macrolide, both bind to the 50S ribosomal subunit. Macrolide A, therefore, is the first compound within the macrolide class of antibiotics that exhibits an aminoglycoside mechanism of action.

While the present invention has been described with reference to the specific embodiments thereof it should be understood by those skilled in the art that various changes may be made and equivalents may be substituted without departing from the true spirit and scope of the invention. In addition, many modifications may be made to adopt a particular situation, material, composition of matter, process, process step or steps, to the objective spirit and scope of the present invention. All such modifications are intended to be within the scope of the claims appended hereto.

What is claimed is:

1. A synthetic, biologically active mangrolide A compound.

2. The synthetic, biologically active mangrolide A compound of claim 1, wherein the compound is of structural Formula 14.5:

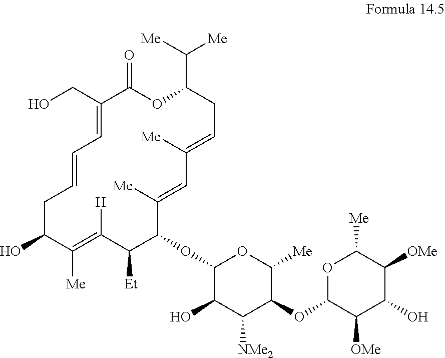 [SMILES: CC[C@H]1/C=C(\C)[C@@H](O)C/C=C/C=C(\CO)C(=O)O[C@H](C(C)C)C/C=C(C)/C=C(\C)[C@@H]1O[C@@H]1O[C@H](C)[C@@H](O[C@@H]2O[C@H](C)[C@@H](OC)[C@H](O)[C@H]2OC)[C@H](N(C)C)[C@H]1O]

3. A method for treating a disease or a disorder caused by a bacterial infection in a patient, comprising administering to the patient a therapeutic amount of the synthetic compound of any one of claims 1 and 2.

4. The method according to claim 3, wherein the synthetic compound according to claim 1 exhibits antibacterial activity against a population of Gram-negative bacteria.

5. The method according to claim 4, wherein the antibacterial activity is a bacteriocidal activity or bacteriostatic activity.

6. The method according to claim 3, wherein the therapeutic amount of the synthetic compound according to claim 1 is effective against a multidrug resistant bacterial infection.

7. The method according to claim 3, wherein the population of Gram-negative bacteria is a population selected from the group consisting of Enterococcus faecalis, Escherichia coli, Klebsiella pneunomiae, Burkholderia cepacia, and Pseudomonas aeruginosa.

8. The method according to claim 7, wherein the population of Gram-negative bacteria is selected from the group consisting of Pseudomonas aeruginosa and Burkholderia cepacia.

9. A pharmaceutical composition comprising a therapeutic amount of at least one synthetic compound according to any one of claims 1 and 2 and a pharmaceutically acceptable carrier.

10. A method for the synthesis of a fragment of Formula 1.6 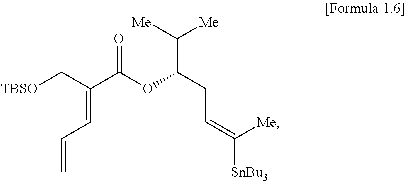
or a pharmaceutically acceptable salt thereof required for synthesis of a correct mangrolide Aglycon disilylether comprising, in order:
(a) reacting a compound of Formula 1.1: 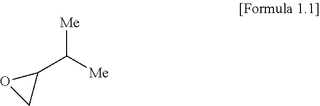
with acetic acid, toluence in the presence of a (R, R)-Salen-Co (II) catalyst at room temperature to form a compound of Formula 1.2: 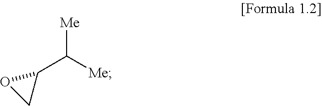
(b) reacting the compound of formula 1.2 in the presence of 1-propyne, n-butyllithium (n-BuLi) boron trifluoride diethyl etherate (BF₃OEt₂), and tetrahydrofuran (THF) at −78° C. to form a compound of Formula 1.3: 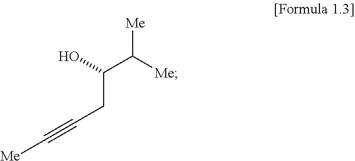
(c) reacting the compound of formula 1.3 in the presence of tributyl tin hydride (Bu₃SnH, which catalyzes the deoxygenation of alcohols), copper (I) cyanide (CuCN), n-butyllithium (n-BuLi), methanol, and tetrahydrofuran (THF), at −78° C. to −20° C., to form a compound of Formula 1.4, 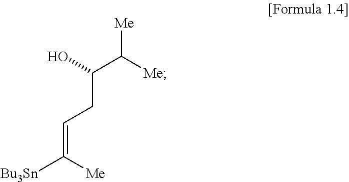
and
(d) reacting the compound of formula 1.4 comprising an SnBu₃ protecting group, and a compound of Formula 1.5 comprising a tert-butyl dimethyl silyl (TBSO) protecting group, 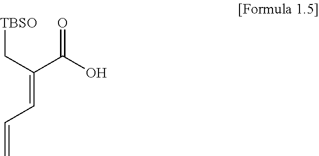 [SMILES: C=C/C=C(\CO[Si](C)(C)C(C)(C)C)C(=O)O]
in the presence of diphenyl phosphate chlorate (PhO)₂P(O)Cl), triethylamine (NEt₃), and 4-dimethyl aminopyridine (DMAP), a nucleophilic catalyst for esterification, in toluene at 45° C. to form a compound of Formula 1.6:

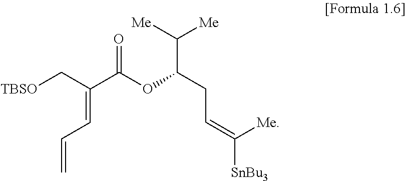

11. A method for the synthesis of a compound of Formula 7.11: 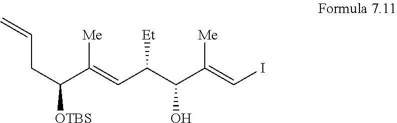 [SMILES: C=CC[C@H](O[Si](C)(C)C(C)(C)C)/C(C)=C/[C@H](CC)[C@@H](O)/C(C)=C/I]
comprising in order:
(a) Reacting a compound of formula 2.1 in the presence of 3,4-dihydropyran, pyridinium p-toluene sulfonate (PPTS) and dichloromethane (CH₂Cl₂) to form a compound of Formula 2.2: 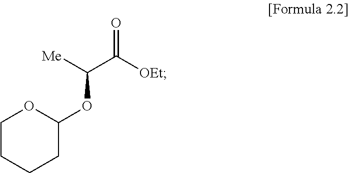 [SMILES: CCOC(=O)[C@H](C)OC1CCCCO1]
(b) Reacting the compound of Formula 2.2 in the presence of diisobutylaluminum hydride (DIBAL) amd dichloroethane (CH₂Cl₂) at −78° C. to form a compound of Formula 2.3: 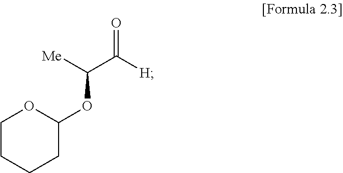
(c) Reacting the compound of Formula 2.3 in the presence of n-PrPh₃PBr, n-butyllilthium (nBuLi), tetrohydrofuran (THF) at −78° C. to room temperature to form a compound of formula 2.4: 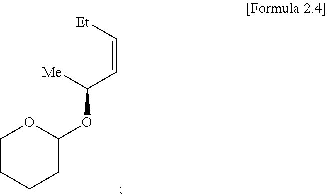 [SMILES: CC/C=C\[C@H](C)OC1CCCCO1]
(d) Reacting the compound of Formula 2.4 with tosylic acid (TsOH.H₂)), ethylene glycol (HO(CH₂)2OH at 0° C. to form a compound of Formula 2.5: 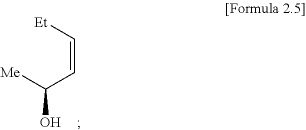
(e) Reacting the compound of Formula 2.5 with a compound of formula J 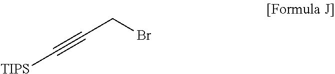
in the presence of sodium tert-butoxide (t-BuONa) and tetrahydrofuran (THF) at 0° C. to form a compound of Formula 2.6; 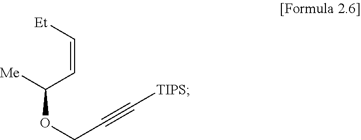
(f) Reacting the compound of Formula 2.6 in the presence of n-butyllithium, tetrahydrofuran (THF) at −78° C. and then methoxy methylether chloride (MOMCl) to form a compound of Formula 7.1: 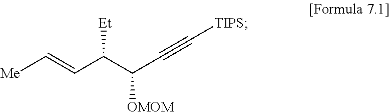
(g) Reacting the compound of Formula 7.1 in the presence of ozone (O3), dichloromethane (CH₂Cl₂) at −78° C.; then dimethylsulfide (Me₂S) at room temperature to form a compound of Formula 7.2: 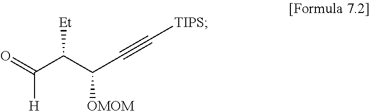
(h) Reacting the compound of Formula 7.2 in the presence of Ph₃P═C(Me)CO₂Et and toluene at 90° C. to form a compound of Formula 7.3: 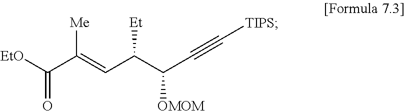
(i) Reacting the compound of Formula 7.3 in the presence of diisobuutylaluminum hydride (DIBAL) and dichloromethane (CH₂Cl₂) at −78° C. to form a compound of Formula 7.4: 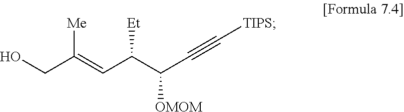
(j) Reacting the compound of Formula 7.4 in the presence of tetra-n-butylammonium fluoride (TBAF) and tetrahydrofuran (THF) at 0° C. to form a compound of Formula 7.5: 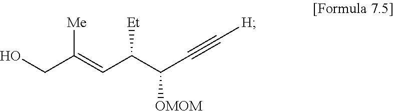
(k) Reacting the compound of Formula 7.5 in the presence of triethylsilyltrifluoromethane sulfonate (TESOTf), and 2,6-lutidine at 0° C. to form a compound of Formula 7.6: 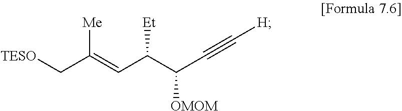
(l) reacting the compound of Formula 7.6 in the presence of tributyltin hydride (Bu₃SnSnBu₃), butyllithium (BuLi), copper (I) cyanide (CuCN), tetrahydrofuran (THF), then methyl iodide (MeI) at −78° C. to room temperature, then elemental iodine (I₂) and dichloromethane (CH₂Cl₂) at 0° C. to form a compound of Formula 7.7: 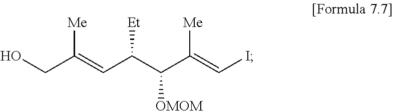
(m) reacting the compound of Formula 7.7 in the presence of Dess-Martin periodinane and dichloromethane (CH₂Cl₂) at 0° C. to form a compound of Formula 7.8: 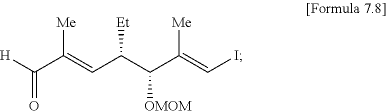
(n) reacting the compound of Formula 7.8 in the presence of (−)-lpc₂B-Allyl diethylether at −78° C. to form the compound of Formula 7.9: 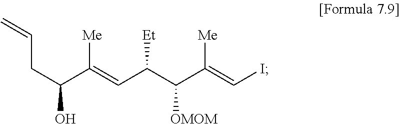
(o) reacting the compound of Formula 7.9 in the presence of triethylsilyltrifluoromethane sulfonate (TESOTf), and 2,6-lutidine at 0° C. to form a compound of Formula 7.10: 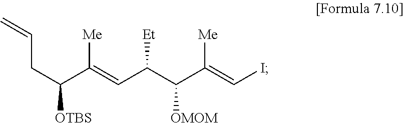
and
(p) reacting the compound of Formula 7.10 in the presence of boron trifluoride etherate (BF₃.OEt₂ and dimethyl sulfide to form a compound of Formula 7.11:

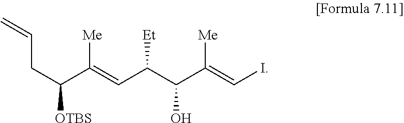 [SMILES: C=CC[C@H](O[Si](C)(C)C(C)(C)C)/C(C)=C/[C@H](CC)[C@@H](O)/C(C)=C/I]

12. A method for the synthesis of a compound of Formula 8.5: 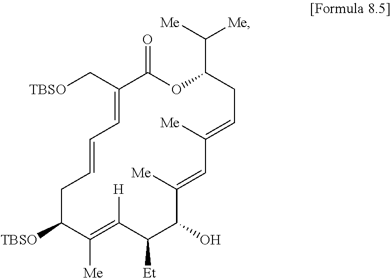 [SMILES: CC[C@H]1/C=C(\C)[C@@H](O[Si](C)(C)C(C)(C)C)C/C=C/C=C(\CO[Si](C)(C)C(C)(C)C)C(=O)O[C@H](C(C)C)C/C=C(C)/C=C(\C)[C@@H]1O]
wherein the compound of Formula 8.5 is synthetic mangrolide Aglycon disilyl ether of correct structure,
comprising, in order:
(a) reacting a compound of Formula 1.6 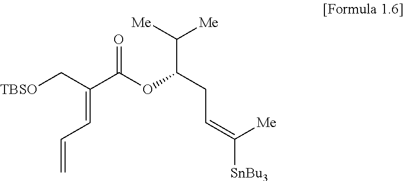
and a compound of Formula 7.11 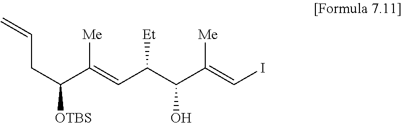 [SMILES: C=CC[C@H](O[Si](C)(C)C(C)(C)C)/C(C)=C/[C@H](CC)[C@@H](O)/C(C)=C/I]
in the presence of the catalyst tetrakis(triphenylphosphine)palladium (Pd(PPh₃)₄), CuCl, LiCl, and dimethylsulfoxide (DMSO) at 70° C. to form a compound of Formula 8.4: 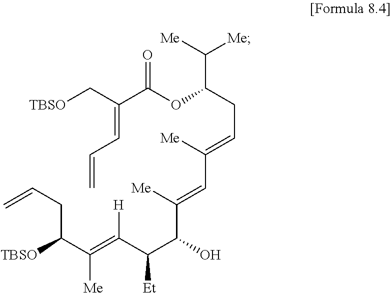 [SMILES: C=C/C=C(\CO[Si](C)(C)C(C)(C)C)C(=O)O[C@@H](C/C=C(C)/C=C(\C)[C@H](O)[C@H](/C=C(\C)[C@H](CC=C)O[Si](C)(C)C(C)(C)C)CC)C(C)C]
and
(b) reacting the compound of formula 8.4 in the presence of Grubbs 2^{nd} catalyst and dichloromethane (CH₂Cl₂) under reflux conditions to form the compound of Formula 8.5:

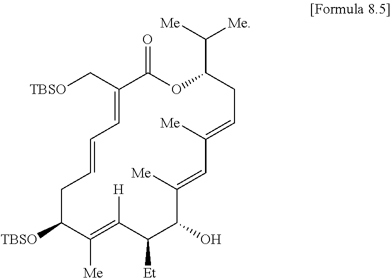 [SMILES: CC[C@H]1/C=C(\C)[C@@H](O[Si](C)(C)C(C)(C)C)C/C=C/C=C(\CO[Si](C)(C)C(C)(C)C)C(=O)O[C@H](C(C)C)C/C=C(C)/C=C(\C)[C@@H]1O]

13. A method for the synthesis of a mangrolide A deoxyglucose fragment a method for the synthesis of a mangrolide A deoxyglucose fragment of Formula 10.9: 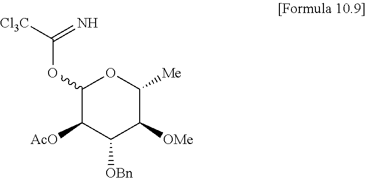
comprising, in order:
(a) reacting a compound of Formula 10.1 in the presence of p-toluenesulfonyl chloride (TsCl), Pyridine (Py) and dichloromethane (CH₂Cl₂) to form a compound of formula 10.2: 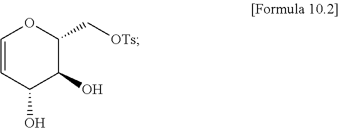 [SMILES: Cc1ccc(S(=O)(=O)OC[C@H]2OC=C[C@@H](O)[C@@H]2O)cc1]
(b) reacting the compound of Formula 10.2 in the presence of lithium aluminum hydride (LiAlH₄) and tetrahydrofuran (THF) to form a compound of Formula 10.3: 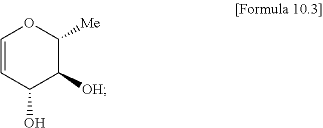
(c) reacting the compound of Formula 10.3 in the presence of dibenzyl tin (II) oxide (Bn₂SnO), and then tetrabutylammonium iodide (TBAI) and benzyl bromide (BnBr) to form a compound of Formula 10.4: 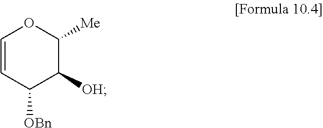 [SMILES: C[C@H]1OC=C[C@@H](OCc2ccccc2)[C@@H]1O]
(d) reacting the compound of formula 10.4 in the presence of potassium bis(trimethylsilyl)amide (KHMDS), methyl iodide (MeI) and tetrahydrofuran (THF) at −78° C. to form a compound of Formula 10.5: 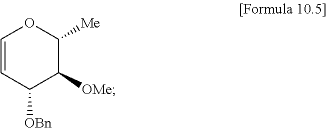 [SMILES: CO[C@@H]1[C@@H](C)OC=C[C@H]1OCc1ccccc1]
(e) reacting the compound of Formula 10.5 in the presence of osmium tetroxide (OsO₄), N-methylmorpholine-N-oxide (NMO), and tert-butanol/acetone/water (1:1:1) at room temperature to form a compound of Formula 10.6: 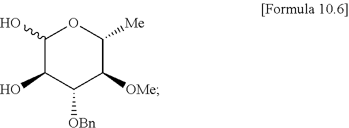 [SMILES: CO[C@H]1[C@H](OCc2ccccc2)[C@@H](O)C(O)O[C@@H]1C]
(f) reacting the compound of Formula 10.6 in the presence of acetic anhydride (Ac₂O), triethylamine (Et3N), 4-(dimethylamino)pyridine (DMAP), and dichloromethane (CH₂Cl₂) to form a compound of Formula 10.7: 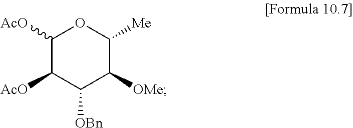 [SMILES: CO[C@H]1[C@H](OCc2ccccc2)[C@@H](OC(C)=O)C(OC(C)=O)O[C@@H]1C]
(g) reacting the compound of Formula 10.7 in the presence of hydrazine acetate and dimethylformamide (DMF) to form a compound of Formula 10.8: 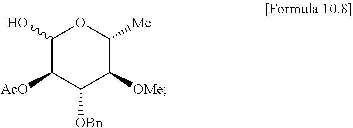 [SMILES: CO[C@H]1[C@H](OCc2ccccc2)[C@@H](OC(C)=O)C(O)O[C@@H]1C]
and
(h) reacting the compound of Formula 10.8 in the presence of 1,8-diazabicycloundec-7-ene (DBU), trichloroacetonitrile (CCl₃CN) and dichloromethane (CH₂Cl₂) to form the compound of Formula 10.9:

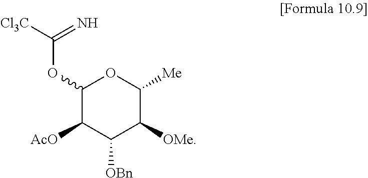

14. A method for the synthesis of a mangrolide A mycaminose sugar of Formula 11.3: 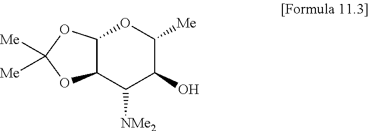
comprising, in order:
(a) reacting a compound of Formula 11.1: 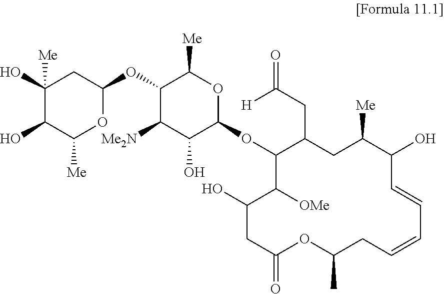 [SMILES: COC1C(O)CC(=O)O[C@H](C)C/C=C\C=C\C(O)[C@H](C)CC(CC=O)C1O[C@@H]1O[C@H](C)[C@@H](O[C@@H]2C[C@](C)(O)[C@H](O)[C@@H](C)O2)[C@H](N(C)C)[C@H]1O]
in the presence of 2N HCl under reflux conditions, to form a compound of Formula 11.2: 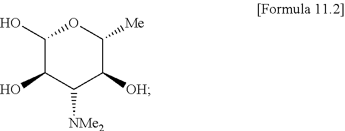
and
(b) reacting the compound of formula 11.2 in the presence of 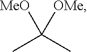
pyridinium toluene-4-sulfonate (PPTS) and dimethylformamide (DMF) to form the compound of Formula 11.3:

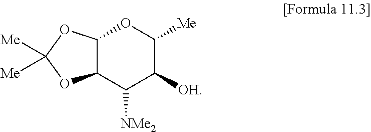

15. A method for the synthesis of a disachharide fragment of mangrolide A of Formula 12.6: 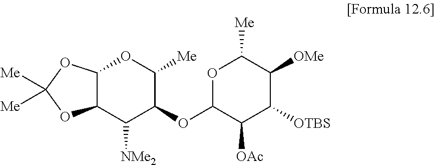
comprising, in order:
(a) reacting a compound of Formula 10.3: 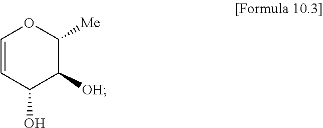
in the presence of tert-butyldimethylsilyl chloride (TBSCl), imidazone, and dichloromethane (CH₂Cl₂) to form a compound of Formula 12.1: 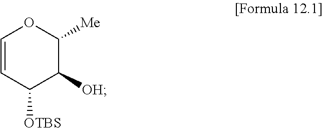 [SMILES: C[C@H]1OC=C[C@@H](O[Si](C)(C)C(C)(C)C)[C@@H]1O]
(b) reacting the compound of Formula 12.1 in the presence of potassium bis(trimethylsilyl)amide (KHMDS), methyl iodide (MeI) and tetrahydrofuran (THF) at −78° C. to form a compound of Formula 12.2: 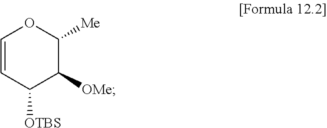 [SMILES: CO[C@@H]1[C@@H](C)OC=C[C@H]1O[Si](C)(C)C(C)(C)C]
(c) reacting the compound of Formula 12.2 in the presence of osmium tetroxide (OsO₄), N-methylmorpholine-N-oxide (NMO), and tert-butanol/acetone/water (1:1:1) at room temperature to form a compound of Formula 12.3: 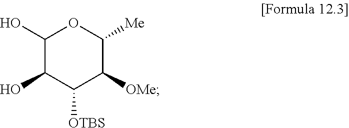 [SMILES: CO[C@H]1[C@H](O[Si](C)(C)C(C)(C)C)[C@@H](O)C(O)O[C@@H]1C]
(d) reacting the compound of Formula 12.3 in the presence of acetic anhydride (Ac2O), triethylamine (Et3N), 4-(dimethylamino)pyridine (DMAP), and dichloromethane (CH2Cl2) to form a compound of Formula 12.4: 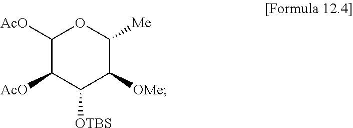 [SMILES: CO[C@H]1[C@H](O[Si](C)(C)C(C)(C)C)[C@@H](OC(C)=O)C(OC(C)=O)O[C@@H]1C]
(e) reacting the compound of Formula 12.4 with a compound of Formula 11.3: 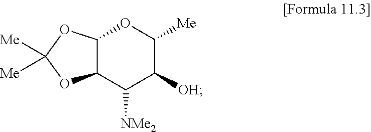
in the presence of tin (IV) chloride (SnCl4) at −15° C. to room temperature to form a compound of Formula 12.5: 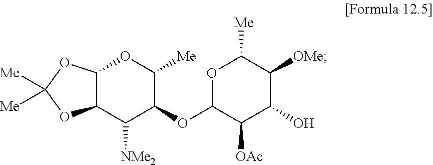
and
(f) reacting the compound of Formula 12.5 in the presence of trifluoromethylsilyl trifluoromethane sulfonate (TBSOTf) and 2,6-lutidine to form the compound of Formula 12.6:

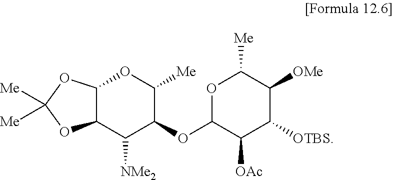

16. A method for the synthesis of a compound of Formula 13.8 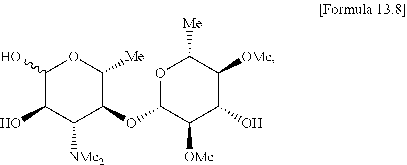 [SMILES: CO[C@H]1[C@H](O[C@H]2[C@H](N(C)C)[C@@H](O)C(O)O[C@@H]2C)O[C@H](C)[C@@H](OC)[C@@H]1O]
wherein the compound of Formula 13.8 matches the degraded sugar fragment from natural mangrolide A,
comprising, in order
(a) reacting a compound of Formula 10.3: 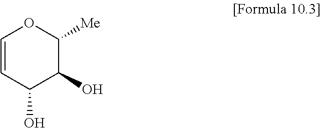
in the presence of acetylchloride (AcCl), pyridine (Py) and dichloromethane (CH₂Cl₂) to form a compound of Formula 13.1: 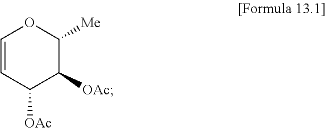 [SMILES: CC(=O)O[C@@H]1[C@@H](C)OC=C[C@H]1OC(C)=O]
(b) reacting the compound of Formula 13.1 in the presence of benzyl alcohol (BnOH), boron trifluoride etherate (BF3.OEt2) at 0° C. to form a compound of Formula 13.2: 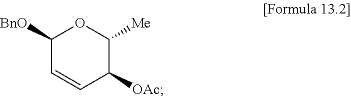 [SMILES: CC(=O)O[C@H]1C=C[C@@H](OCc2ccccc2)O[C@@H]1C]
(c) reacting the compound of Formula 13.2 in the presence of potassium carbonate (K2CO3) and methanol (MeOH) to form a compound of Formula 13.3: 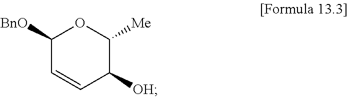 [SMILES: C[C@H]1O[C@H](OCc2ccccc2)C=C[C@@H]1O]
(d) reacting the compound of Formula 13.3 in the presence of meta-clhoroperoxybenzoic acid (mCPBA), sodium bicarbonate (NaHCO3), and dichlromethane (CH₂Cl₂) to form a compound of Formula 13.4: 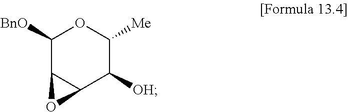 [SMILES: C[C@H]1O[C@H](OCc2ccccc2)[C@@H]2O[C@@H]2[C@@H]1O]
(e) reacting the compound of Formula 13.4 with a compound of Formula 10.9: 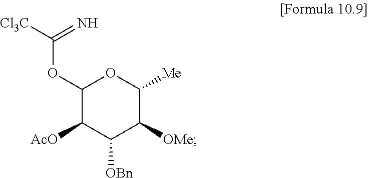
in the presence of boron trifluoride etherate, and dichloromethane at −15 C to form a compound of Formula 13.5: 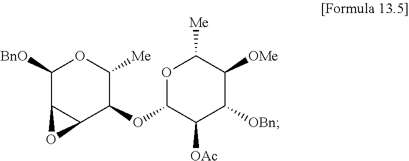 [SMILES: CO[C@H]1[C@H](OCc2ccccc2)[C@@H](OC(C)=O)[C@H](O[C@H]2[C@H]3O[C@H]3[C@@H](OCc3ccccc3)O[C@@H]2C)O[C@@H]1C]
(f) reacting the compound of formula 13.5 in the presence of potassium hydroxide (KOH), DMSO then methyl iodide (MEI) to form a compound of Formula 13.6: 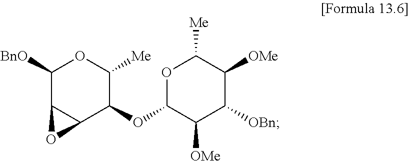
(g) reacting the compound of formula 13.6 in the presence of dimethylamine (Me2NH (aq)) and acetonitrile (MeCN) at 70° C. to form the compound of Formula 13.7: 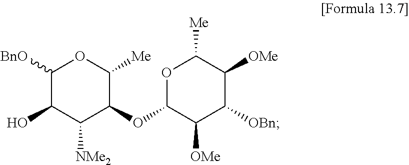
and
(h) reacting the compound of Formula 13.7 in the presence of palladium on carbon (Pd/C), hydrogen gas (H₂), 1N HCl and ethanol (EtOH) to form the compound of Formula 13.8:

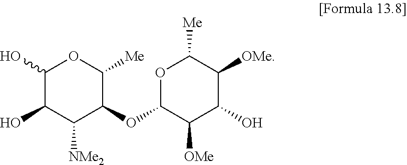 [SMILES: CO[C@H]1[C@H](O[C@H]2[C@H](N(C)C)[C@@H](O)C(O)O[C@@H]2C)O[C@H](C)[C@@H](OC)[C@@H]1O]

17. A method for the synthesis of a fully synthetic, biologically active mangrolide A of Formula 14.5: 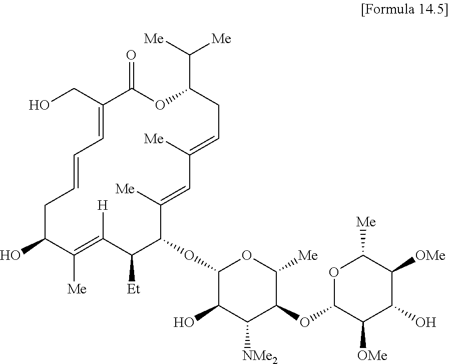 [SMILES: CC[C@H]1/C=C(\C)[C@@H](O)C/C=C/C=C(\CO)C(=O)O[C@H](C(C)C)C/C=C(C)/C=C(\C)[C@@H]1O[C@@H]1O[C@H](C)[C@@H](O[C@@H]2O[C@H](C)[C@@H](OC)[C@H](O)[C@H]2OC)[C@H](N(C)C)[C@H]1O]
comprising, in order
(a) reacting a compound of Formula 13.8: 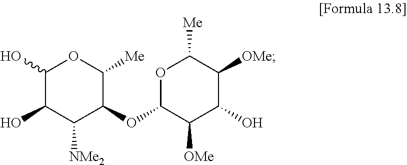
in the presence of aceyic anhydride (Ac₂O), pyridine (Py), 4-dimethylaminopyridine (DMAP), and dichloromethane (CH₂Cl₂) to form a compound of Formula 14.1: 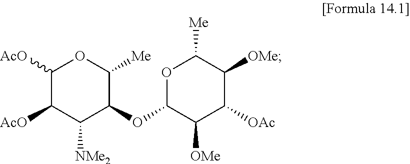 [SMILES: CO[C@H]1[C@H](O[C@H]2[C@H](N(C)C)[C@@H](OC(C)=O)C(OC(C)=O)O[C@@H]2C)O[C@H](C)[C@@H](OC)[C@@H]1OC(C)=O]
(b) reacting the compound of Formula 14.1 in the presence of thiophenol (PhSH) and Boron trifluoride diethyl etherate (BF₃OEt₂) to form a compound of Formula 14.2: 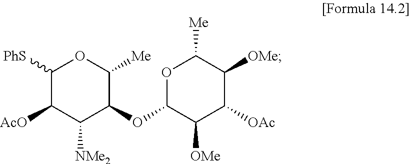
(c) reacting the compound of Formula 14.2 and a compound of Formula 8.5: 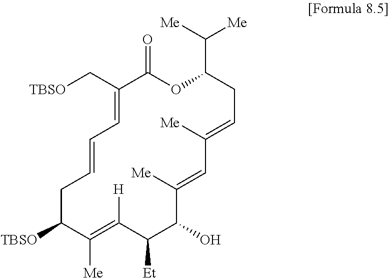 [SMILES: CC[C@H]1/C=C(\C)[C@@H](O[Si](C)(C)C(C)(C)C)C/C=C/C=C(\CO[Si](C)(C)C(C)(C)C)C(=O)O[C@H](C(C)C)C/C=C(C)/C=C(\C)[C@@H]1O]
in the presence of n-iodosuccinimide (NIS) and trimethylsilyl trifluoromethanesulfonate (TMSOf) to form a compound of Formula 14.3: 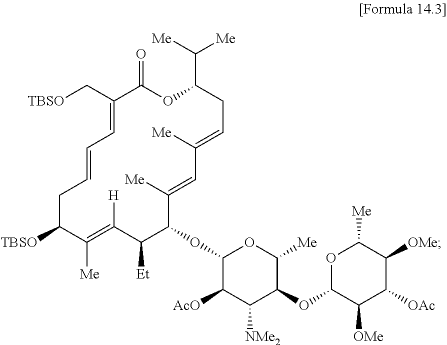 [SMILES: CC[C@H]1/C=C(\C)[C@@H](O[Si](C)(C)C(C)(C)C)C/C=C/C=C(\CO[Si](C)(C)C(C)(C)C)C(=O)O[C@H](C(C)C)C/C=C(C)/C=C(\C)[C@@H]1O[C@@H]1O[C@H](C)[C@@H](O[C@@H]2O[C@H](C)[C@@H](OC)[C@H](OC(C)=O)[C@H]2OC)[C@H](N(C)C)[C@H]1OC(C)=O]
(d) reacting the compound of Formula 14.3 in the presence of potassium carbonate (K₂CO₃) and methanol (MeOH) to form a compound of Formula 14.4: 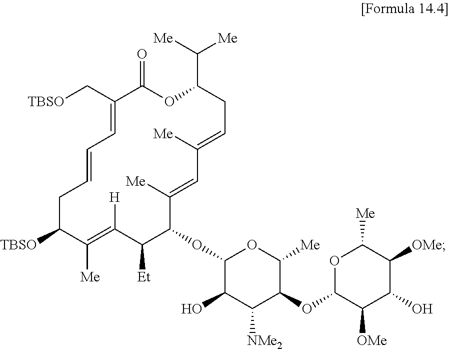
and
(e) reacting the compound of Formula 14.4 in the presence of hydrofluoric acid (HF), pyridine (Py) and tetrahydrofuran (THF) to form the compound of Formula 14.5: 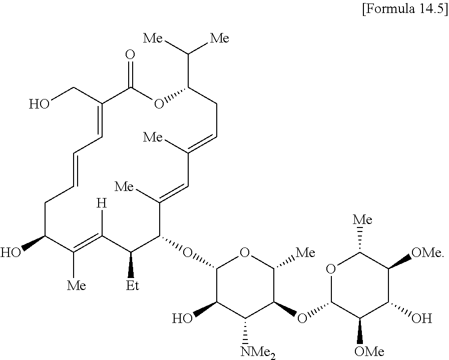 [SMILES: CC[C@H]1/C=C(\C)[C@@H](O)C/C=C/C=C(\CO)C(=O)O[C@H](C(C)C)C/C=C(C)/C=C(\C)[C@@H]1O[C@@H]1O[C@H](C)[C@@H](O[C@@H]2O[C@H](C)[C@@H](OC)[C@H](O)[C@H]2OC)[C@H](N(C)C)[C@H]1O]


SEQUENCE LISTING (1 sequence)

SEQ ID NO: 1 - RNA, 65 nt, Artificial Sequence
  poly(UUU) RNA
gcggcaaggagguaaauaauguuuuuuuuuuuuuuuuuuuuuuuuuuuuuuuuuuuuuaagcagg